SEQUENCE LISTING (154 sequences)

SEQ ID NO: 1 - DNA, 351 bp, Mus Musculus
  168-A5-234-114 Gamma Chain
caggtgcagctgaaggagtcaggacctggcctggtggcgccctcacagagcctgtccatcacatgcactgtctcagggttctcattgaccgactatggtgtgagctgggttcgccagcctccaggaaagggtctggagtggctgggagtaatttggggtgacgggaacacaaattatcattcagctctcatatccagactgagcatcagcaaggataactccaagagccaagtttttttaaaactgaacagtctgcaaactgatgacacagccacctactactgtggcaaatcctttggtgtctatgctatggactactggggtcaaggaacctcagtcaccgtctcctcg

SEQ ID NO: 2 - protein, 117 aa, Mus musculus
  168-A5-234-114 Gamma Chain
QVQLKESGPGLVAPSQSLSITCTVSGFSLTDYGVSWVRQPPGKGLEWLGVIWGDGNTNYHSALISRLSISKDNSKSQVFLKLNSLQTDDTATYYCGKSFGVYAMDYWGQGTSVTVSS

SEQ ID NO: 3 - DNA, 27 bp, Mus musculus
  168-A5-234-114 Gamma Chain CDR1
ttctcattgaccgactatggtgtgagc

SEQ ID NO: 4 - protein, 9 aa, Mus musculus
  168-A5-234-114 Gamma Chain CDR1
FSLTDYGVS

SEQ ID NO: 5 - DNA, 48 bp, Mus musculus
  168-A5-234-114 Gamma Chain CDR2
gtaatttggggtgacgggaacacaaattatcattcagctctcatatcc

SEQ ID NO: 6 - protein, 16 aa, Mus musculus
  168-A5-234-114 Gamma Chain CDR2
VIWGDGNTNYHSALIS

SEQ ID NO: 7 - DNA, 27 bp, Mus musculus
  168-A5-234-114 Gamma Chain CDR3
tcctttggtgtctatgctatggactac

SEQ ID NO: 8 - protein, 9 aa, Mus musculus
  168-A5-234-114 Gamma Chain CDR3
SFGVYAMDY

SEQ ID NO: 9 - DNA, 351 bp, Mus musculus
  168-A5-338-284 Gamma Chain
caggtgcagctgaaggagtcaggacctggcctggtggcgccctcacagagcctgtccatcacatgcactgtctctgggttctctttaagcagttatggtgtacactgggttcgccagtctccaggaaagggtctggagtggctgggagtgatgtggggtgacgggagcacaaattatcactcaggtctcatctccagactgaccatcagcaaggatagttccaagagccaagttttcttaaaactgaacagtctgcaaactgatgacacagccacttactattgtggcaaatcctttggtgtctatgctgtggactactggggtcaaggaacctccgtcaccgtctcctcg

SEQ ID NO: 10 - protein, 117 aa, Mus musculus
  168-A5-338-284 Gamma Chain
QVQLKESGPGLVAPSQSLSITCTVSGFSLSSYGVHWVRQSPGKGLEWLGVMWGDGSTNYHSGLISRLTISKDSSKSQVFLKLNSLQTDDTATYYCGKSFGVYAVDYWGQGTSVTVSS

SEQ ID NO: 11 - DNA, 27 bp, Mus musculus
  168-A5-338-284 Gamma Chain CDR1
ttctctttaagcagttatggtgtacac

SEQ ID NO: 12 - protein, 9 aa, Mus musculus
  168-A5-338-284 Gamma Chain CDR1
FSLSSYGVH

SEQ ID NO: 13 - DNA, 48 bp, Mus musculus
  168-A5-338-284 Gamma Chain CDR2
gtgatgtggggtgacgggagcacaaattatcactcaggtctcatctcc

SEQ ID NO: 14 - protein, 16 aa, Mus musculus
  168-A5-338-284 Gamma Chain CDR2
VMWGDGSTNYHSGLIS

SEQ ID NO: 15 - DNA, 27 bp, Mus musculus
  168-A5-338-284 Gamma Chain CDR3
tcctttggtgtctatgctgtggactac

SEQ ID NO: 16 - protein, 9 aa, Mus musculus
  168-A5-338-284 Gamma Chain CDR3
SFGVYAVDY

SEQ ID NO: 17 - DNA, 321 bp, Mus musculus
  168-A5-234-114 Kappa Chain
gaaatccagatgacacagactacatcctccctgtctgcctctctgggagacagagtcaccatcaattgcaggacaagtcaggacactaacaattatataaactggtatcagcagaaaccagatggaactgttaaactcctgatctactacacatcaatgttacactcaggagtcccatcaaggttcagtggcagtgggtctggaacagattattctctcaccattagcaacctggagcaagaagatattgccacttacttttgccaacagggtgatacgcttcctccgacgttcggtggaggcaccaaggtggaaatcaaa

SEQ ID NO: 18 - protein, 107 aa, Mus musculus
  168-A5-234-114 Kappa Chain
EIQMTQTTSSLSASLGDRVTINCRTSQDTNNYINWYQQKPDGTVKLLIYYTSMLHSGVPSRFSGSGSGTDYSLTISNLEQEDIATYFCQQGDTLPPTFGGGTKVEIK

SEQ ID NO: 19 - DNA, 33 bp, Mus musculus
  168-A5-234-114 Kappa Chain CDR1
aggacaagtcaggacactaacaattatataaac

SEQ ID NO: 20 - protein, 11 aa, Mus musculus
  168-A5-234-114 Kappa Chain CDR1
RTSQDTNNYIN

SEQ ID NO: 21 - DNA, 21 bp, Mus musculus
  168-A5-234-114 Kappa Chain CDR2
tacacatcaatgttacactca

SEQ ID NO: 22 - protein, 7 aa, Mus musculus
  168-A5-234-114 Kappa Chain CDR2
YTSMLHS

SEQ ID NO: 23 - DNA, 27 bp, Mus musculus
  168-A5-234-114 Kappa Chain CDR3
caacagggtgatacgcttcctccgacg

SEQ ID NO: 24 - protein, 9 aa, Mus musculus
  168-A5-234-114 Kappa Chain CDR3
QQGDTLPPT

SEQ ID NO: 25 - DNA, 321 bp, Mus musculus
  168-A5-338-284 Kappa Chain
gatatccagatgacacagactacatcctccctgtctgcctctctgggagacagagtcaccattaattgcagggcaagtcaggacgttaacaattatttaaactggtatcagcagaaaccagatggaactgttaaactcctgatctattacacatcaatgttacactcaggagtcccatcaaggttcagtggcagtgggtctgaaacagattattctctcaccattaccaacctggagcaagaagatattgccacttacttttgccaacagggtgagacgcttcctccgacgttcggtggaggcaccaaggtggaaatcaaa

SEQ ID NO: 26 - protein, 107 aa, Mus musculus
  168-A5-338-284 Kappa Chain
DIQMTQTTSSLSASLGDRVTINCRASQDVNNYLNWYQQKPDGTVKLLIYYTSMLHSGVPSRFSGSGSETDYSLTITNLEQEDIATYFCQQGETLPPTFGGGTKVEIK

SEQ ID NO: 27 - DNA, 33 bp, Mus musculus
  168-A5-338-284 Kappa Chain CDR1
agggcaagtcaggacgttaacaattatttaaac

SEQ ID NO: 28 - protein, 11 aa, Mus musculus
  168-A5-338-284 Kappa Chain CDR1
RASQDVNNYLN

SEQ ID NO: 29 - DNA, 21 bp, Mus musculus
  168-A5-338-284 Kappa Chain CDR2
tacacatcaatgttacactca

SEQ ID NO: 30 - protein, 7 aa, Mus musculus
  168-A5-338-284 Kappa Chain CDR2
YTSMLHS

SEQ ID NO: 31 - DNA, 27 bp, Mus musculus
  168-A5-338-284 Kappa Chain CDR3
caacagggtgagacgcttcctccgacg

SEQ ID NO: 32 - protein, 9 aa, Mus musculus
  168-A5-338-284 Kappa Chain CDR3
QQGETLPPT

SEQ ID NO: 33 - protein, 149 aa, human metapneumo virus
  F-protein sequence for isolate NL/1/00
IGVYGSSVIYMVQLPIFGVIDTPCWIVKAAPSCSGKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCS

SEQ ID NO: 34 - protein, 149 aa, human metapneumo virus
  F-protein sequence for isolate UK/1/00
IGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCS

SEQ ID NO: 35 - protein, 149 aa, human metapneumo virus; identical to SEQ ID NO: 34
  F-protein sequence for isolate NL/2/00

SEQ ID NO: 36 - protein, 149 aa, human metapneumo virus; identical to SEQ ID NO: 34
  F-protein sequence for isolate NL/13/00

SEQ ID NO: 37 - protein, 149 aa, human metapneumo virus; identical to SEQ ID NO: 34
  F-protein sequence for isolate NL/14/00

SEQ ID NO: 38 - protein, 149 aa, human metapneumo virus; identical to SEQ ID NO: 34
  F-protein sequence for isolate FL/3/01

SEQ ID NO: 39 - protein, 149 aa, human metapneumo virus; identical to SEQ ID NO: 34
  F-protein sequence for isolate FL/4/01

SEQ ID NO: 40 - protein, 149 aa, human metapneumo virus; identical to SEQ ID NO: 34
  F-protein sequence for isolate FL/8/01

SEQ ID NO: 41 - protein, 149 aa, human metapneumo virus; identical to SEQ ID NO: 34
  F-protein sequence for isolate UK/1/01

SEQ ID NO: 42 - protein, 149 aa, human metapneumo virus; identical to SEQ ID NO: 34
  F-protein sequence for isolate UK/7/01

SEQ ID NO: 43 - protein, 149 aa, human metapneumo virus; identical to SEQ ID NO: 34
  F-protein sequence for isolate FL/10/01

SEQ ID NO: 44 - protein, 149 aa, human metapneumo virus; identical to SEQ ID NO: 34
  F-protein sequence for isolate NL/6/01

SEQ ID NO: 45 - protein, 149 aa, human metapneumo virus; identical to SEQ ID NO: 34
  F-protein sequence for isolate NL/8/01

SEQ ID NO: 46 - protein, 149 aa, human metapneumo virus
  F-protein sequence for isolate NL/10/01
IGVYGSSVIYMVQLPIFGVIDTPCWIVKAAPSCSEKKGNYACLLREDQRWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCS

SEQ ID NO: 47 - protein, 149 aa, human metapneumo virus; identical to SEQ ID NO: 34
  F-protein sequence for isolate NL/14/01

SEQ ID NO: 48 - protein, 149 aa, human metapneumo virus; identical to SEQ ID NO: 34
  F-protein sequence for isolate NL/20/01

SEQ ID NO: 49 - protein, 149 aa, human metapneumo virus; identical to SEQ ID NO: 34
  F-protein sequence for isolate NL/25/01

SEQ ID NO: 50 - protein, 149 aa, human metapneumo virus; identical to SEQ ID NO: 34
  F-protein sequence for isolate NL/26/01

SEQ ID NO: 51 - protein, 149 aa, human metapneumo virus; identical to SEQ ID NO: 34
  F-protein sequence for isolate NL/28/01

SEQ ID NO: 52 - protein, 149 aa, human metapneumo virus; identical to SEQ ID NO: 34
  F-protein sequence for isolate NL/30/01

SEQ ID NO: 53 - protein, 149 aa, human metapneumo virus; identical to SEQ ID NO: 33
  F-protein sequence for isolate BR/2/01

SEQ ID NO: 54 - protein, 149 aa, human metapneumo virus; identical to SEQ ID NO: 33
  F-protein sequence for isolate BR/3/01

SEQ ID NO: 55 - protein, 149 aa, human metapneumo virus; identical to SEQ ID NO: 34
  F-protein sequence for isolate NL/2/02

SEQ ID NO: 56 - protein, 149 aa, human metapneumo virus; identical to SEQ ID NO: 34
  F-protein sequence for isolate NL/4/02

SEQ ID NO: 57 - protein, 149 aa, human metapneumo virus; identical to SEQ ID NO: 34
  F-protein sequence for isolate NL/5/02

SEQ ID NO: 58 - protein, 149 aa, human metapneumo virus; identical to SEQ ID NO: 34
  F-protein sequence for isolate NL/6/02

SEQ ID NO: 59 - protein, 149 aa, human metapneumo virus; identical to SEQ ID NO: 33
  F-protein sequence for isolate NL/7/02

SEQ ID NO: 60 - protein, 149 aa, human metapneumo virus; identical to SEQ ID NO: 34
  F-protein sequence for isolate NL/9/02

SEQ ID NO: 61 - protein, 149 aa, human metapneumo virus; identical to SEQ ID NO: 33
  F-protein sequence for isolate FL/1/02

SEQ ID NO: 62 - protein, 149 aa, human metapneumo virus; identical to SEQ ID NO: 34
  F-protein sequence for isolate NL/1/81

SEQ ID NO: 63 - protein, 149 aa, human metapneumo virus; identical to SEQ ID NO: 34
  F-protein sequence for isolate NL/1/93

SEQ ID NO: 64 - protein, 149 aa, human metapneumo virus
  F-protein sequence for isolate NL/2/93
IGVYGSSVIYMVQLPIFGVIDTPCWIVKAAPSCSEKKGNYACLLREDQGWYCQNAGSTVYYPNEKDCETRGDHVFCDTVAGINVAEQSKECNINISTTNYPCKVSTGRHPISMVALSPLGALVACYKGVSCSIGSNRVGIIKQLNKGCS

SEQ ID NO: 65 - protein, 149 aa, human metapneumo virus; identical to SEQ ID NO: 34
  F-protein sequence for isolate NL/4/93

SEQ ID NO: 66 - protein, 149 aa, human metapneumo virus; identical to SEQ ID NO: 34
  F-protein sequence for isolate NL/1/95

SEQ ID NO: 67 - protein, 149 aa, human metapneumo virus; identical to SEQ ID NO: 34
  F-protein sequence for isolate NL/2/96

SEQ ID NO: 68 - protein, 149 aa, human metapneumo virus; identical to SEQ ID NO: 34
  F-protein sequence for isolate NL/3/96

SEQ ID NO: 69 - protein, 149 aa, human metapneumo virus; identical to SEQ ID NO: 34
  F-protein sequence for isolate NL/1/98

SEQ ID NO: 70 - protein, 149 aa, human metapneumo virus; identical to SEQ ID NO: 34
  F-protein sequence for isolate NL/17/00

SEQ ID NO: 71 - protein, 149 aa, human metapneumo virus; identical to SEQ ID NO: 34
  F-protein sequence for isolate NL/22/01

SEQ ID NO: 72 - protein, 149 aa, human metapneumo virus; identical to SEQ ID NO: 34
  F-protein sequence for isolate NL/29/01

SEQ ID NO: 73 - protein, 149 aa, human metapneumo virus; identical to SEQ ID NO: 34
  F-protein sequence for isolate NL/23/01

SEQ ID NO: 74 - protein, 149 aa, human metapneumo virus; identical to SEQ ID NO: 34
  F-protein sequence for isolate NL/17/01

SEQ ID NO: 75 - protein, 149 aa, human metapneumo virus; identical to SEQ ID NO: 34
  F-protein sequence for isolate NL/24/01

SEQ ID NO: 76 - protein, 149 aa, human metapneumo virus; identical to SEQ ID NO: 34
  F-protein sequence for isolate NL/3/02

SEQ ID NO: 77 - protein, 149 aa, human metapneumo virus
  F-protein sequence for isolate NL/3/98
IGVYGSSVIYMVQLPIFGVIDTPCWIIKAAPSCSEKNGNYACLLREDQGWYCKNAGSTVYYPNEKDCETRGDHVFCDTAAGINVAEQSRECNINISTTNYPCKVSTGRHPISMVALSPLGALVACYKGVSCSIGSNRVGIIKQLPKGCS

SEQ ID NO: 78 - protein, 149 aa, human metapneumo virus
  F-protein sequence for isolate NL/1/99
IGVYGSSVIYMVQLPIFGVIDTPCWIIKAAPSCSEKNGNYACLLREDQGWYCKNAGSTVYYPNEKDCETRGDHVFCDTAAGINVAEQSRECNINISTTNYPCKVSTGRHPISMVALSPLGALVACYKGVSCSIGSNWVGIIKQLPKGCS

SEQ ID NO: 79 - protein, 149 aa, human metapneumo virus; identical to SEQ ID NO: 77
  F-protein sequence for isolate NL/2/99

SEQ ID NO: 80 - protein, 149 aa, human metapneumo virus; identical to SEQ ID NO: 77
  F-protein sequence for isolate NL/3/99

SEQ ID NO: 81 - protein, 149 aa, human metapneumo virus; identical to SEQ ID NO: 77
  F-protein sequence for isolate NL/11/00

SEQ ID NO: 82 - protein, 149 aa, human metapneumo virus; identical to SEQ ID NO: 77
  F-protein sequence for isolate NL/12/00

SEQ ID NO: 83 - protein, 149 aa, human metapneumo virus; identical to SEQ ID NO: 77
  F-protein sequence for isolate NL/1/01

SEQ ID NO: 84 - protein, 149 aa, human metapneumo virus
  F-protein sequence for isolate NL/5/01
IGVYGSSVIYMVQLPIFGVIDTPCWIIKAAPSCSEKNGNYACLLREDQGWYCKNAGSTVYYPNEKDCETRGDHVFCDTAAGINVAEQSRECNINISTTNYPCKVSTGRHSISMVALSPLGALVACYKGVSCSIGSNRVGIIKQLPKGCS

SEQ ID NO: 85 - protein, 149 aa, human metapneumo virus; identical to SEQ ID NO: 77
  F-protein sequence for isolate NL/9/01

SEQ ID NO: 86 - protein, 149 aa, human metapneumo virus; identical to SEQ ID NO: 77
  F-protein sequence for isolate NL/19/01

SEQ ID NO: 87 - protein, 149 aa, human metapneumo virus; identical to SEQ ID NO: 77
  F-protein sequence for isolate NL/21/01

SEQ ID NO: 88 - protein, 149 aa, human metapneumo virus; identical to SEQ ID NO: 77
  F-protein sequence for isolate UK/11/01

SEQ ID NO: 89 - protein, 149 aa, human metapneumo virus; identical to SEQ ID NO: 77
  F-protein sequence for isolate FL/1/01

SEQ ID NO: 90 - protein, 149 aa, human metapneumo virus; identical to SEQ ID NO: 77
  F-protein sequence for isolate FL/2/01

SEQ ID NO: 91 - protein, 149 aa, human metapneumo virus; identical to SEQ ID NO: 77
  F-protein sequence for isolate FL/5/01

SEQ ID NO: 92 - protein, 149 aa, human metapneumo virus; identical to SEQ ID NO: 77
  F-protein sequence for isolate FL/7/01

SEQ ID NO: 93 - protein, 149 aa, human metapneumo virus; identical to SEQ ID NO: 77
  F-protein sequence for isolate FL/9/01

SEQ ID NO: 94 - protein, 149 aa, human metapneumo virus; identical to SEQ ID NO: 77
  F-protein sequence for isolate UK/10/01

SEQ ID NO: 95 - protein, 149 aa, human metapneumo virus; identical to SEQ ID NO: 77
  F-protein sequence for isolate NL/1/02

SEQ ID NO: 96 - protein, 149 aa, human metapneumo virus
  F-protein sequence for isolate NL/1/94
IGVYGSSVIYMVQLPIFGVIDTPCWIIKAAPSCSEKDGNYACLLREDQGWYCKNAGSTVYYPNEKDCETRGDHVFCDTAAGINVAEQSRECNINISTTNYPCKVSTGRHPISMVALSPLGALVACYKGVSCSIGSNRVGIIKQLPKGCS

SEQ ID NO: 97 - protein, 149 aa, human metapneumo virus; identical to SEQ ID NO: 96
  F-protein sequence for isolate NL/1/96

SEQ ID NO: 98 - protein, 149 aa, human metapneumo virus; identical to SEQ ID NO: 96
  F-protein sequence for isolate NL/6/97

SEQ ID NO: 99 - protein, 149 aa, human metapneumo virus; identical to SEQ ID NO: 96
  F-protein sequence for isolate NL/7/00

SEQ ID NO: 100 - protein, 149 aa, human metapneumo virus; identical to SEQ ID NO: 96
  F-protein sequence for isolate NL/9/00

SEQ ID NO: 101 - protein, 149 aa, human metapneumo virus; identical to SEQ ID NO: 96
  F-protein sequence for isolate NL/19/00

SEQ ID NO: 102 - protein, 149 aa, human metapneumo virus; identical to SEQ ID NO: 96
  F-protein sequence for isolate NL/28/00

SEQ ID NO: 103 - protein, 149 aa, human metapneumo virus; identical to SEQ ID NO: 96
  F-protein sequence for isolate NL/3/01

SEQ ID NO: 104 - protein, 149 aa, human metapneumo virus; identical to SEQ ID NO: 96
  F-protein sequence for isolate NL/4/01

SEQ ID NO: 105 - protein, 149 aa, human metapneumo virus; identical to SEQ ID NO: 96
  F-protein sequence for isolate NL/11/01

SEQ ID NO: 106 - protein, 149 aa, human metapneumo virus; identical to SEQ ID NO: 96
  F-protein sequence for isolate NL/15/01

SEQ ID NO: 107 - protein, 149 aa, human metapneumo virus; identical to SEQ ID NO: 96
  F-protein sequence for isolate NL/18/01

SEQ ID NO: 108 - protein, 149 aa, human metapneumo virus; identical to SEQ ID NO: 96
  F-protein sequence for isolate FL/6/01

SEQ ID NO: 109 - protein, 149 aa, human metapneumo virus; identical to SEQ ID NO: 96
  F-protein sequence for isolate UK/5/01

SEQ ID NO: 110 - protein, 149 aa, human metapneumo virus; identical to SEQ ID NO: 96
  F-protein sequence for isolate UK/8/01

SEQ ID NO: 111 - protein, 149 aa, human metapneumo virus; identical to SEQ ID NO: 96
  F-protein sequence for isolate NL/12/02

SEQ ID NO: 112 - protein, 149 aa, human metapneumo virus; identical to SEQ ID NO: 96
  F-protein sequence for isolate HK/1/02

SEQ ID NO: 113 - protein, 539 aa, human metapneumo virus
  F-protein sequence for HMPV isolate NL/1/00
MSWKVVIIFSLLITPQHGLKESYLEESCSTITEGYLSVLRTGWYTNVFTLEVGDVENLTCADGPSLIKTELDLTKSALRELRTVSADQLAREEQIENPRQSRFVLGAIALGVATAAAVTAGVAIAKTIRLESEVTAIKNALKKTNEAVSTLGNGVRVLATAVRELKDFVSKNLTRAINKNKCDIADLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSGKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAEKGNTGFIIVIILIAVLGSTMILVSVFIIIKKTKKPTGAPPELSGVTNNGFIPHN

SEQ ID NO: 114 - protein, 539 aa, human metapneumo virus
  F-protein sequence for HMPV isolate NL/17/00
MSWKVVIIFSLLITPQHGLKESYLEESCSTITEGYLSVLRTGWYTNVFTLEVGDVENLTCSDGPSLIKTELDLTKSALRELKTVSADQLAREEQIENPRQSRFVLGAIALGVATAAAVTAGVAIAKTIRLESEVTAIKNALKTTNEAVSTLGNGVRVLATAVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYTVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPIKFPEDQFNVALDQVFENIENSQALVDQSNRILSSAEKGNTGFIIVIILIAVLGSSMILVSIFIIIKKTKKPTGAPPELSGVTNNGFIPHS

SEQ ID NO: 115 - protein, 539 aa, human metapneumo virus
  F-protein sequence for HMPV isolate NL/1/99
MSWKVMIIISLLITPQHGLKESYLEESCSTITEGYLSVLRTGWYTNVFTLEVGDVENLTCTDGPSLIKTELDLTKSALRELKTVSADQLAREEQIENPRQSRFVLGAIALGVATAAAVTAGIAIAKTIRLESEVNAIKGALKQTNEAVSTLGNGVRVLATAVRELKEFVSKNLTSAINRNKCDIADLKMAVSFSQFNRRFLNVVRQFSDNAGITPAISLDLMTDAELARAVSYMPTSAGQIKLMLENRAMVRRKGFGILIGVYGSSVIYMVQLPIFGVIDTPCWIIKAAPSCSEKNGNYACLLREDQGWYCKNAGSTVYYPNEKDCETRGDHVFCDTAAGINVAEQSRECNINISTTNYPCKVSTGRHPISMVALSPLGALVACYKGVSCSIGSNWVGIIKQLPKGCSYITNQDADTVTIDNTVYQLSKVEGEQHVIKGRPVSSSFDPIKFPEDQFNVALDQVFESIENSQALVDQSNKILNSAEKGNTGFIIVVILVAVLGLTMISVSIIIIIKKTRKPTGAPPELNGVTNGGFIPHS

SEQ ID NO: 116 - protein, 539 aa, human metapneumo virus
  F-protein sequence for HMPV isolate NL/1/94
MSWKVMIIISLLITPQHGLKESYLEESCSTITEGYLSVLRTGWYTNVFTLEVGDVENLTCTDGPSLIKTELDLTKSALRELKTVSADQLAREEQIENPRQSRFVLGAIALGVATAAAVTAGIAIAKTIRLESEVNAIKGALKTTNEAVSTLGNGVRVLATAVRELKEFVSKNLTSAINKNKCDIADLKMAVSFSQFNRRFLNVVRQFSDNAGITPAISLDLMTDAELARAVSYMPTSAGQIKLMLENRAMVRRKGFGILIGVYGSSVIYMVQLPIFGVIDTPCWIIKAAPSCSEKDGNYACLLREDQGWYCKNAGSTVYYPNEKDCETRGDHVFCDTAAGINVAEQSRECNINISTTNYPCKVSTGRHPISMVALSPLGALVACYKGVSCSIGSNRVGIIKQLPKGCSYITNQDADTVTIDNTVYQLSKVEGEQHVIKGRPVSSSFDPIRFPEDQFNVALDQVFESIENSQALVDQSNKILNSAEKGNTGFIIVIILIAVLGLTMISVSIIIIIKKTRKPTGAPPELNGVTNGGFIPHS

SEQ ID NO: 117 - DNA, 37 bp, Artificial Sequence
  Primer used to construct truncated soluble fusion protein from NL\1\00 (SFnl\1\00)
aaccaaaagcttcaccatgtcttggaaagtggtgatc

SEQ ID NO: 118 - DNA, 54 bp, Artificial Sequence
  Primer used to construct truncated soluble fusion protein from NL\1\00 (SFnl\1\00)
ttaattgaattcttagtgatggtgatggtgatggccagtgtttcctttctctgc

SEQ ID NO: 119 - DNA, 40 bp, Artificial sequence
  Primer used to construct truncated soluble fusion protein from NL\1\99 (SFnl\1\99)
ttccttaagcttcaccatgtcttggaaagtgatgatcatc

SEQ ID NO: 120 - DNA, 57 bp, Artificial Sequence
  Primer used to construct truncated soluble fusion protein from NL\1\99 (SFnl\1\99)
ttaattggatccttagtgatggtgatggtgatgaccagtgtttcctttttctgcact

SEQ ID NO: 121 - DNA, 33 bp, Artificial sequence
  Primer used to construct truncated soluble fusion protein from NL\1\00 (SFnl\1\00) and soluble fusion protein NL\1\99 (SFnl\1\99)
aatcaacggtccgccaccatgtcttggaaagtg

SEQ ID NO: 122 - DNA, 54 bp, Artificial Sequence; identical to SEQ ID NO: 118
  Primer used to construct truncated soluble fusion protein from NL\1\00 (SFnl\1\00) and soluble fusion protein NL\1\99 (SFnl\1\99)

SEQ ID NO: 123 - DNA, 37 bp, Artificial sequence; identical to SEQ ID NO: 117
  Oligonucleotide Primer used to construct Full length F-protein (NL\1\00)

SEQ ID NO: 124 - DNA, 35 bp, Artificial Sequence
  Oligonucleotide Primer used to construct Full length F-protein (NL\1\00)
aattaaggatcctaattatgtggtatgaagccatt

SEQ ID NO: 125 - DNA, 40 bp, Artificial Sequence; identical to SEQ ID NO: 119
  Oligonucleotide Primer used to construct Full length F-protein (NL\1\99)

SEQ ID NO: 126 - DNA, 35 bp, Artificial Sequence
  Oligonucleotide Primer used to construct Full length F-protein (NL\1\99)
aattaaggatcctaattatgtggtatgaaaccgcc

SEQ ID NO: 127 - protein, 50 aa, Artificial Sequence
  hMPVf001 - fragments of F protein of hMPV isolate
LMTDAELARAVSNMPTSAGQIKLMLENRAMVRRKGFGILIGVYGSSVIYM

SEQ ID NO: 128 - protein, 50 aa, human metapneumovirus; identical to SEQ ID NO: 127
  hMPVf991 - fragments of F protein of hMPV isolate

SEQ ID NO: 129 - protein, 50 aa, human metapneumovirus
  hMPVf941 - fragments of F protein of hMPV isolate
LMTDAELARAVSYMPTSAGQIKLMLENRAMVRRKGFGILIGVYGSSVIYM

SEQ ID NO: 130 - protein, 50 aa, human metapneumovirus; identical to SEQ ID NO: 129
  hMPVf0017 - fragments of F protein of hMPV isolate

SEQ ID NO: 131 - protein, 50 aa, Respiratory Syncytial Virus
  RSVF-long A - fragments of F protein of RSV isolate
LMTDAELARAVSNMPTSAGQIKLMLENRAMVRRKGFGILIGVYGSSVIYT

SEQ ID NO: 132 - protein, 24 aa, RSV (Respiratory Syncytial Virus)
  fragments of the F protein
NSELLSLINDMPITNDQKKLMSNN

SEQ ID NO: 133 - protein, 24 aa, RSV (Respiratory Syncytial Virus)
  fragments of the F protein
NSELLSLINDMPITNDQKRLMSNN

SEQ ID NO: 134 - protein, 24 aa, RSV (Respiratory Syncytial Virus)
  fragments of the F protein
NSELLSLINDMPITNDQKQLMSNN

SEQ ID NO: 135 - protein, 24 aa, RSV (Respiratory Syncytial Virus)
  fragments of the F protein
NSELLSLINDMPITNDQKTLMSNN

SEQ ID NO: 136 - protein, 24 aa, RSV (Respiratory Syncytial Virus)
  fragments of the F protein
NSELLSLINDMPITNDQKELMSNN

SEQ ID NO: 137 - protein, 24 aa, RSV (Respiratory Syncytial Virus)
  fragments of the F protein
NSELLSLINDMPITNDQKDLMSNN

SEQ ID NO: 138 - protein, 24 aa, RSV (Respiratory Syncytial Virus)
  fragments of the F protein
NSELLSLINDMPITNDQKMLMSNN

SEQ ID NO: 139 - protein, 24 aa, RSV (Respiratory Syncytial Virus)
  fragments of the F protein
NSELLSLINDMPITNDQKHLMSNN

SEQ ID NO: 140 - protein, 24 aa, RSV (Respiratory Syncytial Virus)
  fragments of the F protein
NSELLSLIQDMPITNDQKKLMSNN

SEQ ID NO: 141 - protein, 24 aa, RSV (Respiratory Syncytial Virus)
  fragments of the F protein
NSELLSLIYDMPITNDQKKLMSNN

SEQ ID NO: 142 - protein, 24 aa, RSV (Respiratory Syncytial Virus)
  fragments of the F protein
NSELLSLIKDMPITNDQKKLMSNN

SEQ ID NO: 143 - protein, 24 aa, RSV (Respiratory Syncytial Virus)
  fragments of the F protein
NSELLSLIDDMPITNDQKKLMSNN

SEQ ID NO: 144 - protein, 24 aa, RSV (Respiratory Syncytial Virus)
  fragments of the F protein
NSELLSLIHDMPITNDQKKLMSNN

SEQ ID NO: 145 - protein, 24 aa, RSV (Respiratory Syncytial Virus)
  fragments of the F protein
NSELLSLIRDMPITNDQKKLMSNN

SEQ ID NO: 146 - protein, 24 aa, RSV (Respiratory Syncytial Virus)
  fragments of the F protein
NSELLSLISDMPITNDQKKLMSNN

SEQ ID NO: 147 - protein, 24 aa, RSV (Respiratory Syncytial Virus)
  fragments of the F protein
NSELLSLIADMPITNDQKKLMSNN

SEQ ID NO: 148 - protein, 24 aa, RSV (Respiratory Syncytial Virus)
  fragments of the F protein
NSELSSLINDMPITNDQKKLMSNN

SEQ ID NO: 149 - protein, 24 aa, RSV (Respiratory Syncytial Virus)
  fragments of the F protein
NSELLSLINDMPITNDQKYLMSNN

SEQ ID NO: 150 - protein, 24 aa, RSV (Respiratory Syncytial Virus)
  fragments of the F protein
NSELLSLINDMPITIDQKKLMSNN

SEQ ID NO: 151 - protein, 24 aa, RSV (Respiratory Syncytial Virus)
  fragments of the F protein
NSELLSLINDMPITNDQKNLMSNN

SEQ ID NO: 152 - protein, 24 aa, RSV (Respiratory Syncytial Virus)
  fragments of the F protein
NSELLSLINDMPITNDQKKLMFNN

SEQ ID NO: 153 - protein, 24 aa, RSV (Respiratory Syncytial Virus)
  fragments of the F protein
NSELLSLINDMPITNDQKKLMSEN

SEQ ID NO: 154 - protein, 24 aa, RSV (Respiratory Syncytial Virus)
  fragments of the F protein
NSELLSLINDMPITNDQKKLMSYN

(19) United States
(12) Patent Application Publication
(10) Pub. No.: US 2006/0228367 A1
(43) Pub. Date: Oct. 12, 2006

(54) Antibodies against mammalian metapneumovirus

(75) Inventors: Nancy Ulbrandt, Gaitherburg, MD (US); JoAnn Suzich, Washington Grove, MD (US); Kathleen L. Coelingh, St. Helena, CA (US)

Correspondence Address: JONES DAY, 222 EAST 41ST ST, NEW YORK, NY, 10017, US

(73) Assignee: MedImmune, Inc.

(21) Appl. No.: 11/355,568

(22) Filed: Feb. 15, 2006

Related U.S. Application Data
(60) Provisional application No. 60/669,939, filed on Apr. 8, 2005.

(51) Int. Cl. A61K 39/42; C12Q 1/70

(52) U.S. Cl. 424/159.1; 435/5; 530/388.3

(57) ABSTRACT

The present invention provides antibodies that immunospecifically bind to a polypeptide of a mammalian metapneumovirus, compositions comprising said antibodies, and methods for producing such antibodies. In particular, the invention provides monoclonal antibodies that immunospecifically bind to the F protein of human metapneumovirus and that neutralize human metapneumovirus. The invention also provides antibodies that cross-react with both the F protein of a mammalian metapneumovirus and the F protein of a mammalian respiratory syncytial virus and that neutralize both viruses. Further, the invention provides recombinant antibodies, such as humanized antibodies, against mammalian metapneumovirus, and methods for producing such recombinant antibodies. The invention further provides methods for treating, managing, ameliorating symptoms of and/or preventing infections with mammalian metapneumovirus, such as human metapneumovirus. The invention also provides antibodies that immunospecifically bind the F protein of avian pneumovirus. Antibodies that immunospecifically bind the F protein of avian pneumovirus are useful in the diagnosis and treatment of infections with avian pneumovirus.

The present application claims benefit under 35 U.S.C. §119(e) of U.S. patent application No. 60/669,939 filed Apr. 8, 2005, which is incorporated herein by reference in its entirety.

### 1. FIELD OF THE INVENTION

The present invention provides antibodies that immunospecifically bind to a polypeptide of a mammalian metapneumovirus, compositions comprising said antibodies, and methods for producing such antibodies. In particular, the invention provides monoclonal antibodies that immunospecifically bind to the F protein of human metapneumovirus and that neutralize human metapneumovirus. The invention also provides antibodies that cross-react with both the F protein of a mammalian metapneumovirus and the F protein of a mammalian respiratory syncytial virus and that neutralize both viruses. Further, the invention provides recombinant antibodies, such as humanized or fully human antibodies, against mammalian metapneumovirus, and methods for producing such recombinant antibodies. The invention further provides methods for treating, managing, ameliorating symptoms of and/or preventing infections with mammalian metapneumovirus, such as human metapneumovirus. The invention also provides antibodies that immunospecifically bind the F protein of avian pneumovirus. Antibodies that immunospecifically bind the F protein of avian pneumovirus are useful in the diagnosis and treatment of infections with avian pneumovirus.

### 2. BACKGROUND OF THE INVENTION

Respiratory Syncytial Virus, Avian & Mammalian Metapneumovirus

Respiratory viruses account for a large proportion of upper and lower respiratory tract illness in humans. In the past few decades, many etiological agents of respiratory tract illness have been identified. Of these, respiratory syncytial virus (RSV) is the single most important cause of respiratory infections during infancy and early childhood (Welliver, 2003, J. Pediatr. 143:S112-S117). However, only 60% of clinically attended respiratory infections of infants and children are of a known etiology (Sinaniotis, 2004, Paediatr Respiratory Rev. 5:S197-S200). Recently, a new member of the Paramyxoviridae family has been isolated from 28 children with clinical symptoms reminiscent of those caused by human respiratory syncytial virus (“hRSV”) infection, ranging from mild upper respiratory tract disease to severe bronchiolitis and pneumonia (Van Den Hoogen et al., 2001, Nature Medicine 7:719-724). The new virus was named human metapneumovirus (HMPV) based on sequence homology and gene constellation. The study further showed that by the age of five years virtually all children in the Netherlands have been exposed to hMPV and that the virus has been circulating in humans for at least half a century. Additionally, the seasonality of the infection is similar to RSV, peaking in the winter months (Robinson, 2005, J. Med. Virol. 76:98-105; Williams, 2004, New Engl. J. Med. 350:443-450). However, unlike RSV, hMPV can be isolated year-round, albeit at a lower rate (Robinson, 2005, J. Med. Virol. 76:98-105; Williams, 2004, New Engl. J. Med. 350:443-450). Risk factors for hMPV infection are also similar to those found for RSV. Highest incidence of infection with human metapneumovirus has been found in young children, in the elderly and immunocompromised humans. Infection with human metapneumovirus is a significant burden of disease in at-risk premature infants, chronic lung disease of prematurity, congestive heart disease, and immunodeficiency (Robinson, 2005, J. Med. Virol. 76:98-105; Williams, 2004, New Engl. J. Med. 350:443-450).

The genomic organization of human metapneumovirus is described in van den Hoogen et al., 2002, Virology 295:119-132. Human metapneumovirus has recently been isolated from patients in North America (Peret et al., 2002, J. Infect. Diseases 185:1660-1663).

hMPV shares a similar genetic structure to RSV but lacks the non-structural genes found in RSV (van den Hoogen, 2002, Virology. 295:119-132). Both viruses code for similar surface proteins that are defined as the surface glycoprotein (G) protein and the fusion (F) protein. Based upon differences between the amino acid sequences of the G and F proteins, both RSV and hMPV have been subdivided into A and B groups. However, in hMPV there is a further bifurcation of A and B subgroups into A1, A2, B1, and B2 groupings (Boivin, 2004, Emerg. Infect. Dis. 10:1154-1157, 25). For both RSV and hMPV viruses, the sequences of the G proteins display a wide variance between subgroups; with hMPV the G protein has only 30% identity between A and B subgroups. For both RSV and hMPV the F protein is more conserved; across the known hMPV isolates the F protein amino acid sequence is 95% conserved (Biacchesi, 2003, Virology 315:1-9; Boivin, 2004, Emerg. Infect. Dis.10:1154-1157; van den Hoogen, 2004, Emerg. Infect. Dis. 10:658-666). Despite the similarities in structure of the viruses, the F proteins of hMPV and RSV share only a 33% amino acid sequence identity and antisera generated against either RSV or hMPV do not neutralize across the pneumoviridae group (Wyde, 2003, Antiviral Research. 60:51-59). With RSV a single monoclonal antibody directed at the fusion (F) protein can prevent severe lower respiratory tract RSV infection. Similarly, because of the high level of sequence conservation of the F protein across all the hMPV subgroups, this protein is likely to be the preferred antigenic target for the generation of cross-subgroup neutralizing antibodies.

Human metapneumovirus is related to avian metapneumovirus. For example, the F protein of hMPV is highly homologous to the F protein of avian pneumonovirus (“APV”). Alignment of the human metapneumoviral F protein with the F protein of an avian pneumovirus isolated from Mallard Duck shows 85.6% identity in the ectodomain. Alignment of the human metapneumoviral F protein with the F protein of an avian pneumovirus isolated from Turkey (subgroup B) shows 75% identity in the ectodomain. See, e.g., co-owned and co-pending Provisional Application No. 60/358,934, entitled “Recombinant Parainfluenza Virus Expression Systems and Vaccines Comprising Heterologous Antigens Derived from Metapneumovirus,” filed on Feb. 21, 2002, by Haller and Tang, which is incorporated herein by reference in its entirety.

Respiratory disease caused by an APV was first described in South Africa in the late 1970s (Buys et al., 1980, Turkey 28:36-46) where it had a devastating effect on the turkey industry. The disease in turkeys was characterized by sinusitis and rhinitis and was called turkey rhinotracheitis (TRT). The European isolates of APV have also been strongly implicated as factors in swollen head syndrome (SHS) in chickens (O'Brien, 1985, Vet. Rec. 117:619-620). Originally, the disease appeared in broiler chicken flocks infected with Newcastle disease virus (NDV) and was assumed to be a secondary problem associated with Newcastle disease (ND). Antibody against European APV was detected in affected chickens after the onset of SHS (Cook et al., 1988, Avian Pathol. 17:403-410), thus implicating APV as the cause.

The avian pneumovirus is a single stranded, non-segmented RNA virus that belongs to the sub-family Pneumovirinae of the family Paramyxoviridae, genus metapneumovirus (Cavanagh and Barrett, 1988, Virus Res. 11:241-256; Ling et al., 1992, J. Gen. Virol. 73:1709-1715; Yu et al., 1992, J. Gen. Virol. 73:1355-1363). The Paramyxoviridae family is divided into two sub-families: the Paramyxovirinae and Pneumovirinae. The subfamily Paramyxovirinae includes, but is not limited to, the genera: Paramyxovirus, Rubulavirus, and Morbillivirus. Recently, the sub-family Pneumovirinae was divided into two genera based on gene order, i.e., pneumovirus and metapneumovirus (Naylor et al., 1998, J. Gen. Virol., 79:1393-1398; Pringle, 1998, Arch. Virol. 143:1449-1159). The pneumovirus genus includes, but is not limited to, human respiratory syncytial virus (hRSV), bovine respiratory syncytial virus (bRSV), ovine respiratory syncytial virus, and mouse pneumovirus. The metapneumovirus genus includes, but is not limited to, European avian pneumovirus (subgroups A and B), which is distinguished from hRSV, the type species for the genus pneumovirus (Naylor et al., 1998, J. Gen. Virol., 79:1393-1398; Pringle, 1998, Arch. Virol. 143:1449-1159). The US isolate of APV represents a third subgroup (subgroup C) within metapneumovirus genus because it has been found to be antigenically and genetically different from European isolates (Seal, 1998, Virus Res. 58:45-52; Senne et al., 1998, In: Proc. 47th WPDC, California, pp. 67-68).

Electron microscopic examination of negatively stained APV reveals pleomorphic, sometimes spherical, virions ranging from 80 to 200 nm in diameter with long filaments ranging from 1000 to 2000 nm in length (Collins and Gough, 1988, J. Gen. Virol. 69:909-916). The envelope is made of a membrane studded with spikes 13 to 15 nm in length. The nucleocapsid is helical, 14 nm in diameter and has 7 nm pitch. The nucleocapsid diameter is smaller than that of the genera Paramyxovirus and Morbillivirus, which usually have diameters of about 18 nm.

Avian pneumovirus infection is an emerging disease in the USA despite its presence elsewhere in the world in poultry for many years. In May 1996, a highly contagious respiratory disease of turkeys appeared in Colorado, and an APV was subsequently isolated at the National Veterinary Services Laboratory (NVSL) in Ames, Iowa (Senne et al., 1997, Proc. 134th Ann. Mtg., AVMA, pp. 190). Prior to this time, the United States and Canada were considered free of avian pneumovirus (Pearson et al., 1993, In: Newly Emerging and Re-emerging Avian Diseases: Applied Research and Practical Applications for Diagnosis and Control, pp. 78-83; Hecker and Myers, 1993, Vet. Rec. 132:172). Early in 1997, the presence of APV was detected serologically in turkeys in Minnesota. By the time the first confirmed diagnosis was made, APV infections had already spread to many farms. The disease is associated with clinical signs in the upper respiratory tract: foamy eyes, nasal discharge and swelling of the sinuses. It is exacerbated by secondary infections. Morbidity in infected birds can be as high as 100%. The mortality can range from 1 to 90% and is highest in six to twelve week old poults.

Avian pneumovirus is transmitted by contact. Nasal discharge, movement of affected birds, contaminated water, contaminated equipment; contaminated feed trucks and load-out activities can contribute to the transmission of the virus. Recovered turkeys are thought to be carriers. Because the virus is shown to infect the epithelium of the oviduct of laying turkeys and because APV has been detected in young poults, egg transmission is considered a possibility.

Based upon the recent work with hMPV, hMPV likewise appears to be a significant factor in human, particularly, juvenile respiratory disease.

Phylogenetic analysis divides the hMPV strains into two genetic clusters, designated subgroups A and B that are distinct from APV viruses (Bastien et al 2003a and b; Biacchesi et al, 2003; Peret et al 2002 and 2004; van den Hoogen, 2002). Within these subgroups, hMPV can be further subdivided into A1, A2, B1, and B2 subtypes (van den Hoogen, 2003).

Complement Determining Region

There are three CDRs in each of the variable regions of the heavy chain and the light chain, which are designated CDR1, CDR2 and CDR3, for each of the variable regions. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers (Chothia & Lesk, J. Mol. Biol. 196:901-917 (1987) and Chothia et al., Nature 342:877-883 (1989)) found that certain sub-portions within Kabat CDRs adopt nearly identical peptide backbone conformations, despite having great diversity at the level of amino acid sequence. These sub-portions were designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262(5):732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Clothia defined CDRs.

### 3. SUMMARY OF THE INVENTION

The present invention provides antibodies that bind immunospecifically to the F protein of a mammalian metapneumovirus, wherein the antibody comprises at least one of the amino acid sequences of SEQ ID NO:2, 4, 6, 8, 10, 12, 14, 16, 18, 20, 22, 24, 26, 28, 30, and 32. The invention also provides fragments of such antibodies. In specific embodiments, the antibodies of the invention are human antibodies, chimeric antibodies or humanized antibodies.

The invention provides methods for generating antibodies that immunospecifically bind to an F protein of a mammalian metapneumovirus.

The invention further provides methods for treating and diagnosing an infection with mammalian metapneumovirus, such as human metapneumovirus, using an antibody of the invention. Pharmaceutical compositions comprising an antibody of the invention and a pharmaceutically acceptable carrier are also provided.

The invention also provides kits, wherein a kit of the invention comprises an antibody of the invention or a fragment of an antibody of the invention.

The invention also provides antibodies that immunospecifically bind to avian pneumovirus.

The invention also provides antibodies that cross-react with both the F protein of a mammalian metapneumovirus and the F protein of a mammalian respiratory syncytial virus and that neutralize both viruses.

3.1 Terminology

As used herein, a “derivative” of a proteinaceous agent (e.g., proteins, polypeptides, peptides, and antibodies) refers to a modified form of the proteinaceous agent, wherein the modification can be one or more of the following: (i) introduction of one or more amino acid residue substitutions; (ii) introduction of one or more deletions; (iii) one or more additions; (iv) the covalent attachment of any type of molecule to the proteinaceous agent resulting in, e.g., glycosylation, acetylation, formylation, pegylation, phosphorylation, amidation, derivatization by known protecting/blocking groups, proteolytic cleavage, linkage to a cellular ligand or other protein; (v) addition of one or more non-classical amino acids; and (vi) substitution with one or more non-classical amino acids. A derivative of a proteinaceous agent may be produced, e.g., by chemical modifications or by recombinant DNA technology.

As used herein, the term “effective amount” refers to the amount of a therapy (e.g., the amount of an antibody of the invention) that is sufficient to reduce and/or ameliorate the severity and/or duration of an infection with mammalian metapneumovirus in a subject, prevent the advancement of an infection with mammalian metapneumovirus, cause regression of an infection with mammalian metapneumovirus, prevent the recurrence, development, or onset of one or more symptoms associated with an infection with mammalian metapneumovirus, or enhance or improve the prophylactic or therapeutic effect(s) of another therapy (e.g., administration of an antiviral agent and/or administration of an agent that strengthens the subject's immune system).

As used herein, the term “human adult” or “adult” refers to a human 18 years of age or older.

As used herein, the terms “human child” or “child” or variations thereof refer to a human between 24 months of age and 18 years of age.

As used herein, the terms “elderly human,” “elderly,” or variations thereof refer to a human 65 years old or older, preferably 70 years old or older.

As used herein, the terms “human infant” or “infant” or variations thereof refer to a human less than 24 months of age, less than 12 months, less than 6 months, less than 3 months, less than 2 months, or less than 1 month of age.

As used herein, the terms “human infant born prematurely,” “preterm infant,” or “premature infant,” or variations thereof refer to a human born at less than 40 weeks of gestational age, less than 35 weeks gestational age, who is less than 6 months old, less than 3 months old, less than 2 months old, or less than 1 month old.

As used herein, the terms “manage,” “managing,” and “management” refer to the beneficial effects that a subject derives from a therapy (e.g., administration of an antibody against mammalian metapneumovirus), which does not result in a cure of the disease, e.g., infection with mammalian metapneumovirus, but allows to prevent the progression or worsening of the disease.

As used herein, the terms “prevent,” “preventing,” and “prevention” refer to the inhibition of the development or onset of a disease or disorder (e.g., an infection with mammalian metapneumovirus) or the prevention of the recurrence, onset, or development of one or more symptoms of such a disease or disorder in a subject resulting from the administration of a therapy (e.g., administration of an antibody against mammalian metapneumovirus).

As used herein, the term “prophylactically effective amount” refers to the amount of a therapy (e.g., administration of an antibody against mammalian metapneumovirus) that is sufficient to result in the prevention of the development, recurrence, or onset of a disease or disorder (e.g., infection with mammalian metapneumovirus).

As used herein, the terms “subject” and “patient” are used interchangeably. As used herein, the terms “subject” and “subjects” refer to an animal, such as a mammal or a bird. Mammals include non-primates (e.g., a cow, pig, horse, cat, dog, rat, and mouse) and primates (e.g., a monkey, such as a cynomolgous monkey, African green monkey, chimpanzee, and a human). Birds include, but are not limited to, turkey, chicken, duck, and goose.

As used herein, the term “therapeutically effective amount” refers to the amount of a therapy (e.g., administration of an antibody against mammalian metapneumovirus), that is sufficient to reduce the severity of a disease or disorder (e.g., infection with a mammalian metapneumovirus), reduce the duration of a respiratory condition, ameliorate one or more symptoms of such a disease or disorder, prevent the advancement of such a disease or disorder, cause regression of such a disease or disorder, or enhance or improve the therapeutic effect(s) of another therapy.

3.2 Abbreviations and Conventions

Abbreviation

hMPV human metapneumovirus
CDR complementarity determining region
K_{D} Dissociation constant
IC₅₀ concentration that is required for 50% inhibition of viral replication in vitro
EC₅₀ concentration required for obtaining 50% of the maximum effect in vivo mAb monoclonal antibody
b/hPIV3 chimeric virus with sequences of human PIV3 and with sequences of bovine PIV3
TCID₅₀ Tissue Culture Infecting Dose
MARM Monoclonal Antibody-Resistant Mutants
APV Avian pneumovirus
mAb234 monoclonal antibody 168-A5-234-114 (mouse hybridoma deposited with the ATCC under deposit no. PTA-6713)
mAb338 monoclonal antibody 168-A5-338-284 (mouse hybridoma deposited with the ATCC under deposit no. PTA-6714)
RSV Respiratory syncytial virus

Names of mutations indicate first the amino acid in the wild type amino acid sequence (single letter code), then the position of the mutated amino acid, and last the mutated amino acid (single letter code).

3.3 Deposit of Biological Material

Mouse hybridoma clone HMPV 168A5.234.114 has been deposited with the American Type Culture Collection (ATCC) as deposit number PTA-6713. And mouse hybridoma clone hMPV 168A5.338.284 has been deposited with the ATCC as deposit number PTA-6714. The address of the ATCC is 10801 University Blvd Manassas, Va. 20110-2209. The deposits were received on May 12, 2005.

### 4. BRIEF DESCRIPTION OF THE DRAWINGS

FIG. 1. Kinetics of 168-A5-234-114 (mAb234) Binding to Soluble F Protein Surfaces

FIG. 2. Kinetics of 168-A5-338-284 (mAb338) Binding to Soluble F Protein Surfaces

FIG. 3. Comparison of the microneutralization of A and B subtypes of virus.

FIG. 4. In Vivo Protection Against NL\1\00 Challenge. The amount and identity of the administered antibody are indicated on the x-axis; lung viral titers and serum IgG concentrations, respectively, are indicated on the y-axis.

FIG. 5. Amino acid sequence alignment of fragments of F proteins of different isolates of hMPV and RSV. Mutants conferring resistance to different monoclonal antibodies are set forth below the alignment. Positions of these mutations are indicated by underlining.

FIG. 6. 168-A5-234-114 (mAb234) Gamma Chain

FIG. 7. 168-A5-338-284 (mAb338) Gamma Chain

FIG. 8. 168-A5-234-114 (mAb234) Kappa Chain

FIG. 9. 168-A5-338-284 (mAb338) Kappa Chain

FIG. 10: Depiction of the epitopes recognized by the monoclonals. Each circle represents an individual epitope with the mAb number shown inside the circle. mAb numbers inside of the intersection of circles are those monoclonals that have recognition sites that are comprised of portion of two epitopes.

FIG. 11. In vivo protection against NL\1\00 challenge. Golden Syrian hamsters were injected 24 hours prior to intranasal challenge with NL\1\00 with varying does of mAb 234 and mAb 338 or with a BSA. Animals were bled prior to challenge to determine the levels of serum antibodies present at time of challenge. At 4 days post infection lungs (panel A) and nasal turbinates (panel B) were harvested and virus titers determined as described in materials and methods. Limit of detection, LOD, for the viral titers was 1.2 log/gm tissue. For IgG quantification in serum samples the sera were diluted 1:100 and 1:500 (panel C). The limit of the quantitation for this assay as performed was 0.1 μg/ml serum. *p=0.008

FIG. 12. In vivo protection against NL\1\99 challenge. Golden Syrian hamsters were injected 24 hours prior to intranasal challenge with NL\1\99 with varying does of mAb 234 and mAb 338 or with a BSA. Animals were bled prior to challenge to determine the levels of serum antibodies present at time of challenge. At 4 days post infection lungs (panel A) and nasal turbinates (panel B) were harvested and virus titers determined as described in materials and methods. Limit of detection, LOD, for the viral titers was 1.2 log/gm tissue. For IgG quantification in serum samples the sera were diluted 1:100 and 1:500 (Panel C). The limit of the quantitation for this assay as performed was 0.1 μg/ml serum. *p=0.0006, ^{#}p<0.0001.

FIG. 13. Comparison of the wild type sequences of the hMPV F protein from NL\1\00 and sequences of F protein derived from monoclonal resistant mutants obtained by selecting hMPV with either mAb 338, mAb 628 or mAb 234. Beneath the sequences of the hMPV F protein is the corresponding homologous region of the RSV F protein from long strain compared to the monoclonal resistant mutant sequences of the F protein selected with Synagis® (palivizumab)(see, Zhao et al., 2004, J. Inf. Dis. 190:1941-1946).

FIG. 14. Comparison of the wild type sequences of the hMPV F protein from NL\1\00 and sequences of F protein derived from monoclonal resistant mutants obtained by selecting hMPV with either mAb 338, mAb 628 or mAb 234. Below the mutant sequences is the wild type sequence of hMPV NL\1\99 which is not neutralized by these antibodies. The corresponding homologous region of RSV F protein is shown to indicate the amino acids in this region shown to elicit an neutralizing response in Corvaisier et al. (Corvaisier, 1997, Arch. Virol. 142: 1073-1086).

FIG. 15. Comparison of the wild type sequences of the hMPV F protein from NL\1\99 and sequences of F protein derived from monoclonal resistant mutants obtained by selecting hMPV with mAb 757. Below is the sequence of the bovine RSV F protein. Underlines and italicized is a region defined by Langedijik et al. as a conserved neutralization motif in the first heptad repeat of the RSV F protein. (Langedijik, 1998, Arch. Virol. 143: 313-320)

FIG. 16. Comparison of the wild type sequences of the hMPV F protein from NL\1\99 and sequences of F protein derived from monoclonal resistant mutants obtained by selecting hMPV with either mAb 836, mAb 710 or mAb 659.

FIG. 17. Cross neutralization of MARMS by hMPV neutralizing antibodies. Monoclonal antibodies (1^{st} column) were tested for their ability to neutralize viral mutants generated by selection with a specific neutralizing antibody (top row). Neutralization phenotype was rated as wild type (WT) if the mAb neutralized the corresponding wild type virus from which the mutant was derived with comparable efficacy. A resistant (R) phenotype was defined as loss of neutralization relative to the neutralization seen with the corresponding wild type virus.

### 5. DETAILED DESCRIPTION OF THE INVENTION

The present invention provides antibodies against mammalian metapneumovirus, such as human metapneumovirus (hMPV). In particular, the invention provides monoclonal antibodies that bind immunospecifically to the F protein of a mammalian metapneumovirus and that have neutralizing activity directed against mammalian metapneumovirus. The invention also provides antibodies that cross-react with both the F protein of a mammalian metapneumovirus and the F protein of a mammalian respiratory syncytial virus and that neutralize both viruses. The invention provides recombinant antibodies against mammalian metapneumovirus and methods for producing such recombinant antibodies. In certain embodiments, the recombinant antibodies of the invention bind to the F protein of a mammalian metapneumovirus, e.g., a human metapneumovirus. In certain aspects, a recombinant antibody of the invention comprises at least one of the CDRs of mAb338 (ATCC deposit no. PTA6714) or mAb234 (ATCC deposit no. PTA6713).

Antibodies of the invention bind immunospecifically to an F protein of a human metapneumovirus. Illustrative F proteins of human metapneumovirus have the amino acid sequence of one of SEQ ID NO:33 to 116. The different F-protein sequences are derived from different viral isolates of human metapneumovirus as set forth in Table 2.

TABLE 2
Origin Of Different F Protein Sequences

| | |
|---|---|
| SEQ ID NO: 33 | F-protein sequence for isolate NL/1/00 |
| SEQ ID NO: 34 | F-protein sequence for isolate UK/1/00 |
| SEQ ID NO: 35 | F-protein sequence for isolate NL/2/00 |
| SEQ ID NO: 36 | F-protein sequence for isolate NL/13/00 |
| SEQ ID NO: 37 | F-protein sequence for isolate NL/14/00 |
| SEQ ID NO: 38 | F-protein sequence for isolate FL/3/01 |
| SEQ ID NO: 39 | F-protein sequence for isolate FL/4/01 |
| SEQ ID NO: 40 | F-protein sequence for isolate FL/8/01 |
| SEQ ID NO: 41 | F-protein sequence for isolate UK/1/01 |
| SEQ ID NO: 42 | F-protein sequence for isolate UK/7/01 |
| SEQ ID NO: 43 | F-protein sequence for isolate FL/10/01 |
| SEQ ID NO: 44 | F-protein sequence for isolate NL/6/01 |
| SEQ ID NO: 45 | F-protein sequence for isolate NL/8/01 |
| SEQ ID NO: 46 | F-protein sequence for isolate NL/10/01 |
| SEQ ID NO: 47 | F-protein sequence for isolate NL/14/01 |
| SEQ ID NO: 48 | F-protein sequence for isolate NL/20/01 |
| SEQ ID NO: 49 | F-protein sequence for isolate NL/25/01 |
| SEQ ID NO: 50 | F-protein sequence for isolate NL/26/01 |
| SEQ ID NO: 51 | F-protein sequence for isolate NL/28/01 |
| SEQ ID NO: 52 | F-protein sequence for isolate NL/30/01 |
| SEQ ID NO: 53 | F-protein sequence for isolate BR/2/01 |
| SEQ ID NO: 54 | F-protein sequence for isolate BR/3/01 |
| SEQ ID NO: 55 | F-protein sequence for isolate NL/2/02 |
| SEQ ID NO: 56 | F-protein sequence for isolate NL/4/02 |
| SEQ ID NO: 57 | F-protein sequence for isolate NL/5/02 |
| SEQ ID NO: 58 | F-protein sequence for isolate NL/6/02 |
| SEQ ID NO: 59 | F-protein sequence for isolate NL/7/02 |
| SEQ ID NO: 60 | F-protein sequence for isolate NL/9/02 |
| SEQ ID NO: 61 | F-protein sequence for isolate FL/1/02 |
| SEQ ID NO: 62 | F-protein sequence for isolate NL/1/81 |
| SEQ ID NO: 63 | F-protein sequence for isolate NL/1/93 |
| SEQ ID NO: 64 | F-protein sequence for isolate NL/2/93 |
| SEQ ID NO: 65 | F-protein sequence for isolate NL/4/93 |
| SEQ ID NO: 66 | F-protein sequence for isolate NL/1/95 |
| SEQ ID NO: 67 | F-protein sequence for isolate NL/2/96 |
| SEQ ID NO: 68 | F-protein sequence for isolate NL/3/96 |
| SEQ ID NO: 69 | F-protein sequence for isolate NL/1/98 |
| SEQ ID NO: 70 | F-protein sequence for isolate NL/17/00 |
| SEQ ID NO: 71 | F-protein sequence for isolate NL/22/01 |
| SEQ ID NO: 72 | F-protein sequence for isolate NL/29/01 |
| SEQ ID NO: 73 | F-protein sequence for isolate NL/23/01 |
| SEQ ID NO: 74 | F-protein sequence for isolate NL/17/01 |
| SEQ ID NO: 75 | F-protein sequence for isolate NL/24/01 |
| SEQ ID NO: 76 | F-protein sequence for isolate NL/3/02 |
| SEQ ID NO: 77 | F-protein sequence for isolate NL/3/98 |
| SEQ ID NO: 78 | F-protein sequence for isolate NL/1/99 |
| SEQ ID NO: 79 | F-protein sequence for isolate NL/2/99 |
| SEQ ID NO: 80 | F-protein sequence for isolate NL/3/99 |
| SEQ ID NO: 81 | F-protein sequence for isolate NL/11/00 |
| SEQ ID NO: 82 | F-protein sequence for isolate NL/12/00 |
| SEQ ID NO: 83 | F-protein sequence for isolate NL/1/01 |
| SEQ ID NO: 84 | F-protein sequence for isolate NL/5/01 |
| SEQ ID NO: 85 | F-protein sequence for isolate NL/9/01 |
| SEQ ID NO: 86 | F-protein sequence for isolate NL/19/01 |
| SEQ ID NO: 87 | F-protein sequence for isolate NL/21/01 |
| SEQ ID NO: 88 | F-protein sequence for isolate UK/11/01 |
| SEQ ID NO: 89 | F-protein sequence for isolate FL/1/01 |
| SEQ ID NO: 90 | F-protein sequence for isolate FL/2/01 |
| SEQ ID NO: 91 | F-protein sequence for isolate FL/5/01 |
| SEQ ID NO: 92 | F-protein sequence for isolate FL/7/01 |
| SEQ ID NO: 93 | F-protein sequence for isolate FL/9/01 |
| SEQ ID NO: 94 | F-protein sequence for isolate UK/10/01 |
| SEQ ID NO: 95 | F-protein sequence for isolate NL/1/02 |
| SEQ ID NO: 96 | F-protein sequence for isolate NL/1/94 |
| SEQ ID NO: 97 | F-protein sequence for isolate NL/1/96 |
| SEQ ID NO: 98 | F-protein sequence for isolate NL/6/97 |
| SEQ ID NO: 99 | F-protein sequence for isolate NL/7/00 |
| SEQ ID NO: 100 | F-protein sequence for isolate NL/9/00 |
| SEQ ID NO: 101 | F-protein sequence for isolate NL/19/00 |
| SEQ ID NO: 102 | F-protein sequence for isolate NL/28/00 |
| SEQ ID NO: 103 | F-protein sequence for isolate NL/3/01 |
| SEQ ID NO: 104 | F-protein sequence for isolate NL/4/01 |
| SEQ ID NO: 105 | F-protein sequence for isolate NL/11/01 |
| SEQ ID NO: 106 | F-protein sequence for isolate NL/15/01 |
| SEQ ID NO: 107 | F-protein sequence for isolate NL/18/01 |
| SEQ ID NO: 108 | F-protein sequence for isolate FL/6/01 |
| SEQ ID NO: 109 | F-protein sequence for isolate UK/5/01 |
| SEQ ID NO: 110 | F-protein sequence for isolate UK/8/01 |
| SEQ ID NO: 111 | F-protein sequence for isolate NL/12/02 |
| SEQ ID NO: 112 | F-protein sequence for isolate HK/1/02 |
| SEQ ID NO: 113 | F protein sequence for HMPV isolate NL/1/00 |
| SEQ ID NO: 114 | F protein sequence for HMPV isolate NL/17/00 |
| SEQ ID NO: 115 | F protein sequence for HMPV isolate NL/1/99 |
| SEQ ID NO: 116 | F protein sequence for HMPV isolate NL/1/94 |

In certain embodiments, an antibody of the invention binds to any F protein of a human metapneumovirus. In other embodiments, an antibody of the invention binds to the F protein of one strain of human metapneumovirus with at least 2-fold, 5-fold, 10-fold, 25-fold, 50-fold, 100-fold, 500-fold, or 1000-fold higher affinity than the antibody binds to the F protein of a different strain of human metapneumovirus.

In certain embodiments, an antibody of the invention, in addition to binding to an F protein of a human MPV and thereby and neutralizing said human MPV, also binds to an F protein of a respiratory syncytial virus (RSV) and neutralizes said RSV. SEQ ID NO:132 to 154 set forth the amino acid sequences of portions of illustrative fragments of F proteins of RSV, wherein such fragments are immunospecifically bound by antibodies against RSV. In certain embodiments, an antibody of the invention binds to an F protein of a mammalian metapneumovirus, such as human metapneumovirus, and to an amino acid sequence of any one of SEQ ID NOs:132-154. In certain embodiments, an antibody of the invention binds to an F protein of a mammalian metapneumovirus, such as human metapneumovirus, and to an amino acid sequence that is at least 80%, 85%, 90%, 95%, 98%, 99%, or at least 99.5%, identical to any one of SEQ ID NOs:132-154.

Antibodies of the invention may further comprise any constant region known in the art, preferably any human constant region known in the art, including, but not limited to, human light chain kappa (κ), human light chain lambda (λ), the constant region of IgG1, the constant region of IgG2, the constant region of IgG3 or the constant region of IgG4.

The present invention provides for pharmaceutical compositions, kits, and articles of manufacture comprising one or more antibodies that immunospecifically binds to an F protein of a mammalian metapneumovirus, such as human metapneumovirus.

The invention also provides methods for producing antibodies that bind to an F protein of a mammalian metapneumovirus using recombinant DNA technology. In certain embodiments, recombinant DNA technology is used to engineer an antibody that has at least one CDR with the amino acid sequence of a CDR of mAb338 or mAb234 or an amino acid sequence that is at least 85%, 90%, 95%, 98%, 99%, or at least 99.5% identical to a CDR of mAb338 or mAb234. In certain embodiments, recombinant DNA technology is used to engineer an antibody that comprises the VH and/or the VL of mAb338 or mAb234. In certain embodiments, recombinant DNA technology is used to engineer an antibody that comprises an amino acid sequence that is at least 85%, 90%, 95%, 98%, 99%, or at least 99.5% identical to the VH and/or the VL of mAb338 or mAb234.

In certain embodiments, the invention also provides antibodies that immunospecifically bind to an F protein of APV. In certain aspects, an antibody that binds immunospecifically to an F protein of APV comprises an amino acid sequence that is at least 85%, 90%, 95%, 98%, or that is 100% identical to one or more of SEQ ID NO:2, 4, 6, 8, 10, 12, 14, 16, 18, 20, 22, 24, 26, 28, 30, or 32. In certain embodiments, an antibody that binds immunospecifically to an F protein of APV comprises the amino acid sequence of one or more complementarity determining regions (“CDRs”) of mAb338 or mAb234. In certain embodiments, an antibody that immunospecifically binds to an F protein of mammalian metapneumovirus as described in section 5.1 also binds to an F protein of APV. Antibodies that immunospecifically bind the F protein of avian pneumovirus are useful in the diagnosis and treatment of infections with avian pneumovirus.

In certain embodiments, an antibody of the invention binds immunospecifically to an F protein of human metapneumovirus and does not cross-react with an F protein of avian metapneumovirus.

In certain embodiments, the invention also provides antibodies that immunospecifically bind to an F protein of RSV. In certain aspects, an antibody that binds immunospecifically to an amino acid sequence that is at least 85%, 90%, 95%, 98%, or that is 100% identical to any one of SEQ ID NOs:132-154, wherein SEQ ID NOs:132-154 are the amino acid sequences of an antigenic region of different F proteins of RSV. In certain embodiments, an antibody that binds immunospecifically to an F protein of RSV comprises the amino acid sequence of one or more complementarity determining regions (“CDRs”) of mAb338 or mAb234. In certain embodiments, an antibody that immunospecifically binds to an F protein of mammalian metapneumovirus as described in section 5.1 also binds to an F protein of RSV.

5.1 Antibodies of the Invention

Antibodies or antibody fragments that can be produced using the methods of the invention include monoclonal antibodies, multispecific antibodies, bispecific antibodies, human antibodies, humanized antibodies, camelised antibodies, chimeric antibodies, single-chain Fvs (scFv), single chain antibodies, single domain antibodies, Fab fragments, F(ab′) fragments, disulfide-linked Fvs (sdFv), and anti-idiotypic (anti-Id) antibodies (including, e.g., anti-Id antibodies to antibodies of the invention), intrabodies, and epitope-binding fragments of any of the above. In particular, antibodies include immunoglobulin molecules and immunologically active fragments of immunoglobulin molecules, i.e., molecules that contain an antigen binding site. Immunoglobulin molecules can be of any type (e.g., IgG, IgE, IgM, IgD, IgA and IgY), class (e.g., IgG1, IgG2, IgG3, IgG4, IgA1 and IgA2) or subclass.

Antibodies or antibody fragments that can be used with the methods of the invention include monoclonal antibodies, multispecific antibodies, human antibodies, humanized antibodies, camelised antibodies, chimeric antibodies, single-chain Fvs (scFv), single chain antibodies, single domain antibodies, Fab fragments, F(ab′) fragments, disulfide-linked Fvs (sdFv), and anti-idiotypic (anti-Id) antibodies (including, e.g., anti-Id antibodies to antibodies of the invention), intrabodies, and epitope-binding fragments of any of the above. In particular, antibodies include immunoglobulin molecules and immunologically active fragments of immunoglobulin molecules, i.e., molecules that contain an antigen binding site. Immunoglobulin molecules can be of any type (e.g., IgG, IgE, IgM, IgD, IgA and IgY), class (e.g., IgG1, IgG2, IgG3, IgG4, IgA1 and IgA2) or subclass.

The present invention provides antibodies that immunospecifically bind to an F protein of a mammalian metapneumovirus, such as a human metapneumovirus. In certain aspects, an antibody of the invention binds to an F protein of a mammalian metapneumovirus with a K_{D} of at most 0.001 nM, 0.005 nM, 0.01 nM, 0.05 nM, 0.1 nM, 0.5 nM, 1 nM, 5 nM, 10 nM, 50 nM, 100 nM, or at most 500 nM. In certain aspects, an antibody of the invention binds to an F protein of a mammalian metapneumovirus with a K_{D} of at least 0.001 nM, 0.005 nM, 0.01 nM, 0.05 nM, 0.1 nM, 0.5 nM, 1 nM, 5 nM, 10 nM, 50 nM, 100 nM, or at least 500 nM. In certain aspects, the K_{D} of an antibody of the invention is between 0.5 nM and 5 nM.

In certain aspects, an antibody of the invention neutralizes mammalian metapneumovirus at an IC₅₀ of at most 0.001 microgram/ml, 0.005 microgram/ml, 0.01 microgram/ml, 0.05 microgram/ml, 0.1 microgram/ml, 0.5 microgram/ml, 1 microgram/ml, 5 microgram/ml, 10 microgram/ml, 50 microgram/ml, 100 microgram/ml, or at most 500 microgram/ml. In certain aspects, an antibody of the invention neutralizes mammalian metapneumovirus at an IC₅₀ of at least 0.001 microgram/ml, 0.005 microgram/ml, 0.01 microgram/ml, 0.05 microgram/ml, 0.1 microgram/ml, 0.5 microgram/ml, 1 microgram/ml, 5 microgram/ml, 10 microgram/ml, 50 microgram/ml, 100 microgram/ml, or at least 500 microgram/ml. In certain aspects, the IC₅₀ of an antibody of the invention for neutralizing mammalian metapneumovirus is between 0.01 microgram/ml and 10 microgram/ml, between 0.01 microgram/ml and 1 microgram/ml, between 0.1 microgram/mil and 1 microgram/ml, between 0.01 microgram/ml and 0.1 microgram/ml, between 0.5 microgram/mil and 5 microgram/mi, or between 0.05 microgram/ml and 2 microgram/ml

In particular, the invention provides the following antibodies that immunospecifically bind to an F protein of a mammalian metapneumovirus: mAb338 (ATCC deposit no. PTA-6714) or mAb234 (ATCC deposit no. PTA-6713). The invention also provides fragments of mAb338 (ATCC deposit no PTA-6714) or mAb234 (ATCC deposit no. PTA-6713), wherein the fragments immunospecifically bind the F protein of a mammalian metapneumovirus.

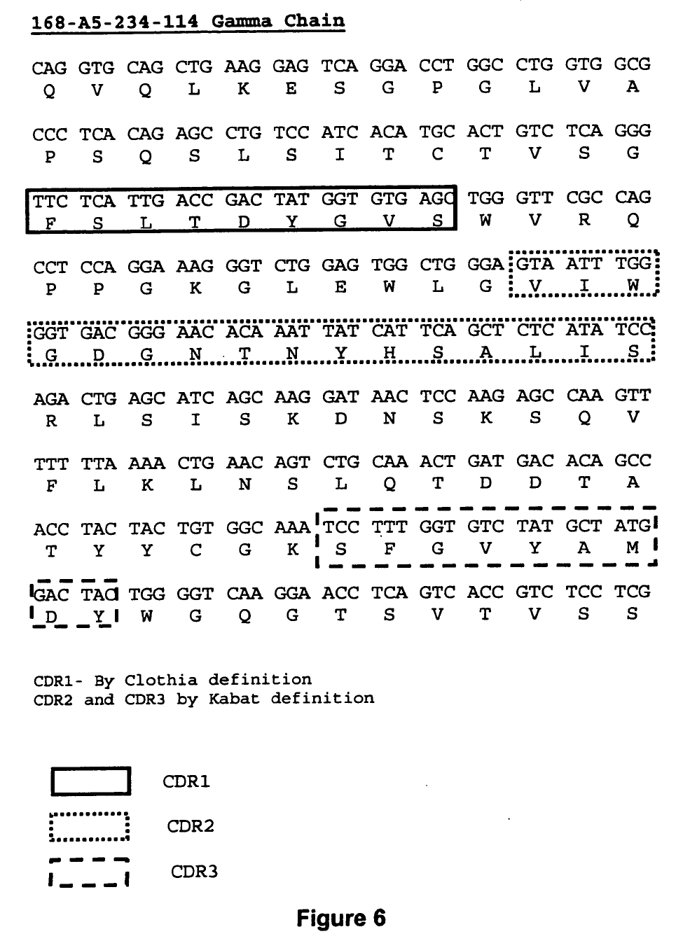

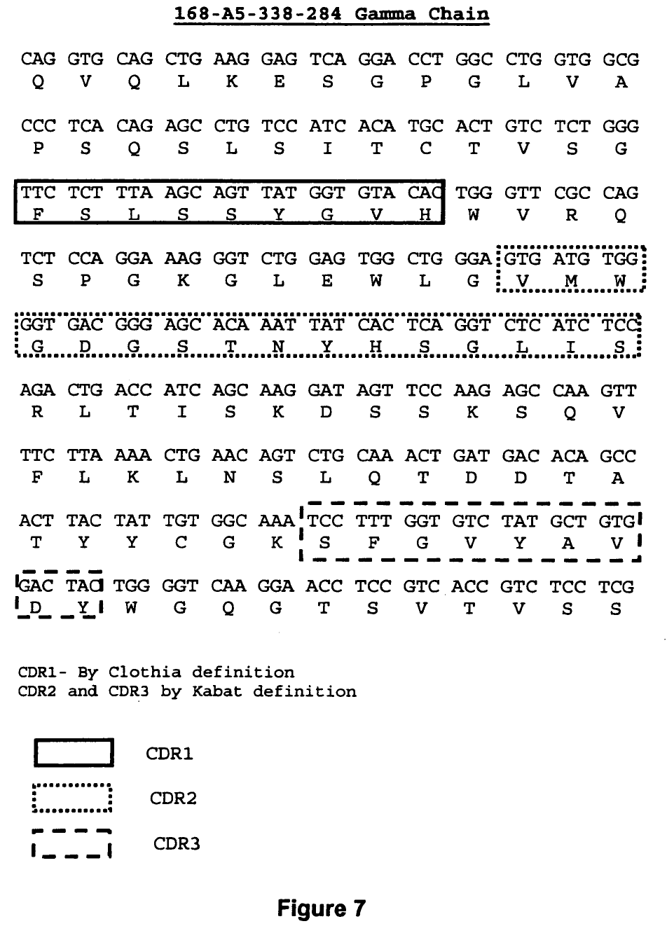

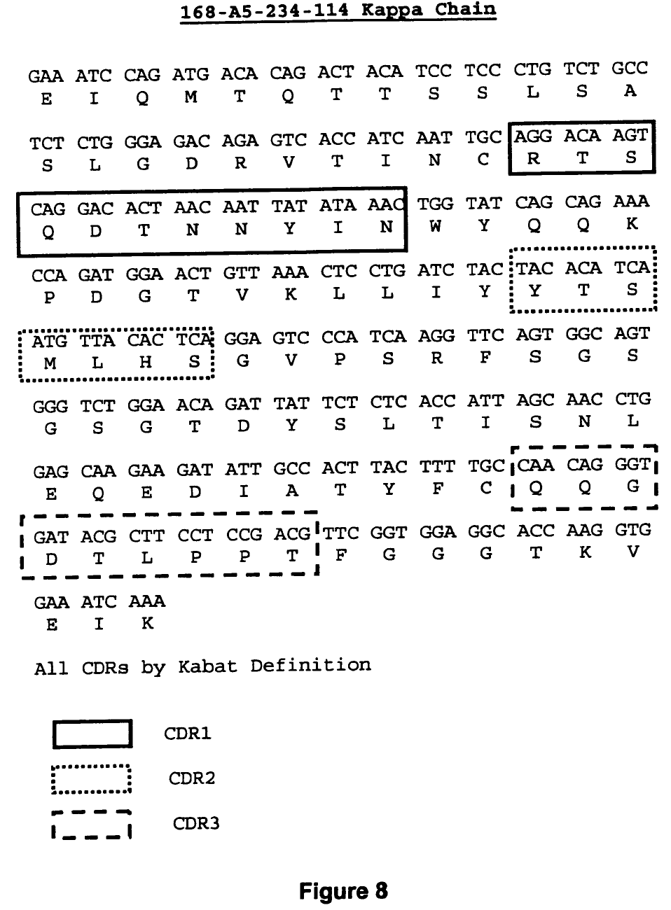

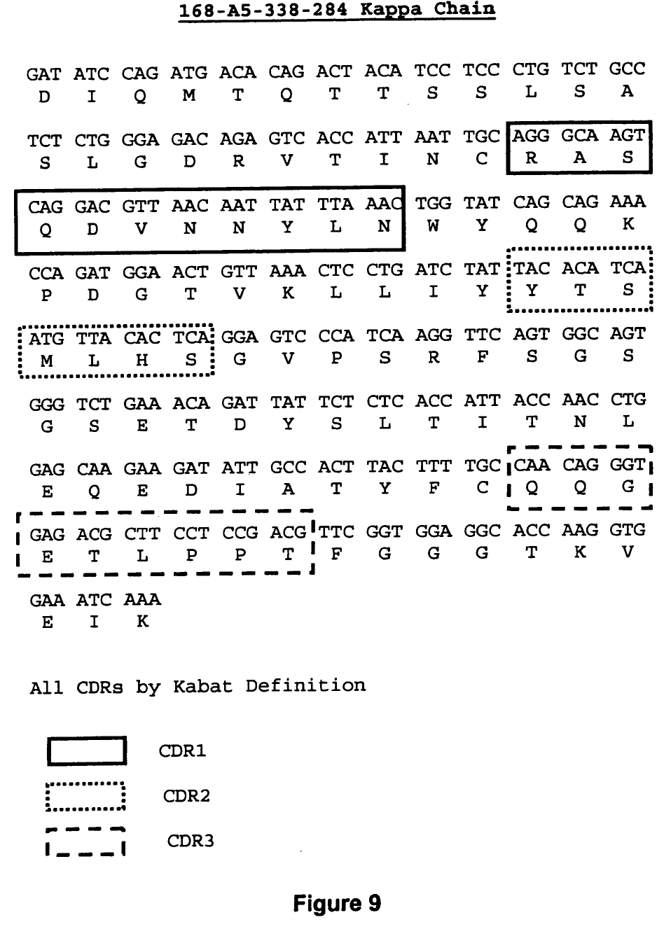

The present invention also provides for antibodies comprising a variable heavy (“VH”) domain and/or a variable light (“VL”) domain having an amino acid sequence of the VH domain and/or VL domain, respectively, of mAb338 (ATCC deposit no. PTA-6714) or mAb234 (ATCC deposit no. PTA-6713). The present invention provides for antibodies comprising one or more complementarity determining regions (“CDRs”) of mAb338 (ATCC deposit no. PTA-6714) or mAb234 (ATCC deposit no. PTA-6713). Sequences of VL and VH of mAb338 and mAb234, respectively, are shown in FIGS. 6-9.

The present invention provides antibodies that immunospecifically bind an F protein of a mammalian metapneumovirus, said antibodies comprising a VH domain having an amino acid sequence of the VH domain of mAb234 (SEQ ID NO.:2), or of the VH domain of mAb338 (SEQ ID NO:10). In certain embodiments, an antibody of the invention comprises a VH domain having an amino acid sequence with at least 85%, 90%, 95%, 98%, 99%, or at least 99.5% identity with the amino acid sequence of the VH domain of mAb234 (SEQ ID NO.:2). In certain embodiments, an antibody of the invention comprises a VH domain having an amino acid sequence with at least 85%, 90%, 95%, 98%, 99%, or at least 99.5% identity with the amino acid sequence of the VH domain of mAb338 (SEQ ID NO.:10).

The present invention provides antibodies that immunospecifically bind an F protein of a mammalian metapneumovirus, said antibodies comprising a VL domain having an amino acid sequence of the VL domain of mAb234 (SEQ ID NO.:18), or of the VL domain of mAb338 (SEQ ID NO:26). In certain embodiments, an antibody of the invention comprises a VL domain having an amino acid sequence with at least 85%, 90%, 95%, 98%, 99%, or at least 99.5% identity with the amino acid sequence of the VL domain of mAb234 (SEQ ID NO.:18). In certain embodiments, an antibody of the invention comprises a VL domain having an amino acid sequence with at least 85%, 90%, 95%, 98%, 99%, or at least 99.5% identity with the amino acid sequence of the VL domain of mAb338 (SEQ ID NO.:26).

In certain embodiments, the invention provides an antibody that immunospecifically binds to the same epitope in an F protein of a mammalian metapneumovirus as mAb234 or mAb338. In certain embodiments, the invention provides an antibody that binds with 2-fold, 5-fold, 10-fold, 25-fold, 50-fold, or 100-fold higher affinity to an epitope in an F protein of a mammalian metapneumovirus than mAb234 or mAb338. In certain specific embodiments, the invention provides an antibody that binds with 2-fold, 5-fold, 10-fold, 25-fold, 50-fold, or 100-fold higher affinity to an epitope in an F protein of a human metapneumovirus that comprises amino acid position 238, 241, and/or 242 of the F protein of a human metapneumovirus. In certain specific embodiments, the invention provides an antibody that binds with 2-fold, 5-fold, 10-fold, 25-fold, 50-fold, or 100-fold higher affinity to an epitope in an F protein of a mammalian metapneumovirus that comprises amino acid position(s) that are homologous to amino acid position 238, 241, and/or 242 in the F protein of a human metapneumovirus. The homologous amino acid positions can be identified by aligning the F protein amino acid sequence of the mammalian metapneumovirus with the amino acid sequence of the F protein of human metapneumovirus.

In certain embodiments, the invention provides methods for identifying antibodies that bind to the F protein of a mammalian metapneumovirus with higher affinity than mAb234 or mAb338. In certain aspects, a competitive binding assay is performed to determine whether a test antibody binds to the F protein of a mammalian metapneumovirus with higher affinity than mAb234 or mAb338. In an illustrative embodiment, mA234 or mA338 is labeled and the test antibody is not labeled. The F protein of a mammalian metapneumovirus is immobilized on a solid surface and incubated with labeled mAb234 or mAb338 and the test antibody under conditions conducive to binding of the antibodies to the F protein. The amount of label that can be detected on the solid support (i.e., the label is attached to the solid support via the F protein and the antibody) is a measure for how much mAb234 or mAb338 is bound to the F protein. The less label is detectable the higher is the affinity of the test antibody compared to mAb234 or mAb338. Any comparative binding test known to the skilled artisan can be used with the methods of the invention.

The present invention provides antibodies that immunospecifically bind to an F protein of a mammalian metapneumovirus, said antibodies comprising a VH CDR having an amino acid sequence of any one of the VH CDRs listed in Table 1, infra, or a VH CDR having an amino acid sequence of at least 85%, 90%, 95%, 98%, 99%, or at least 99.5% identity to any one of the VH CDRs listed in Table 1, infra. In certain embodiments, CDR sequences are deduced using the Kabat or Clothia defmed CDRs.

In particular, the invention provides antibodies that immunospecifically bind to an F protein of a mammalian metapneumovirus, said antibodies comprising (or alternatively, consisting of) one, two, three, four, five or more VH CDRs having an amino acid sequence of any of the VH CDRs listed in Table 1, infra. In one embodiment, an antibody that immunospecifically binds to an F protein of a mammalian metapneumovirus comprises a VH CDR1 having the amino acid sequence of SEQ ID NO.: 4 or SEQ ID NO.: 12. In another embodiment, an antibody that immunospecifically binds to an F protein of a mammalian metapneumovirus comprises a VH CDR2 having the amino acid sequence of SEQ ID NO.: 6 or SEQ ID NO.: 14. In another embodiment, an antibody that immunospecifically binds to an F protein of a mammalian metapneumovirus comprises a VH CDR3 having the amino acid sequence of SEQ ID NO.: 8 or SEQ ID NO.: 16. In another embodiment, an antibody that immunospecifically binds to an F protein of a mammalian metapneumovirus comprises a VH CDR1 having the amino acid sequence of SEQ ID NO.: 4 or SEQ ID NO.: 12 and a VH CDR2 having the amino acid sequence of SEQ ID NO.: 6 or SEQ ID NO.: 14. In another embodiment, an antibody that immunospecifically binds to an F protein of a mammalian metapneumovirus comprises a VH CDR1 having the amino acid sequence of SEQ ID NO.: 4 or SEQ ID NO.: 12 and a VH CDR3 having the amino acid sequence of SEQ ID NO.: 8 or SEQ ID NO.: 16. In another embodiment, an antibody that immunospecifically binds to an F protein of a mammalian metapneumovirus comprises a VH CDR2 having the amino acid sequence of SEQ ID NO.: 6 or SEQ ID NO.: 14 and a VH CDR3 having the amino acid sequence of SEQ ID NO.: 8 or SEQ ID NO.: 16. In another embodiment, an antibody that immunospecifically binds to an F protein of a mammalian metapneumovirus comprises a VH CDR1 having the amino acid sequence of SEQ ID NO.: 4 or SEQ ID NO.: 12, a VH CDR2 having the amino acid sequence of SEQ ID NO.: 6 or SEQ ID NO.: 14, and a VH CDR3 having the amino acid sequence of SEQ ID NO.: 8 or SEQ ID NO.: 16.

The present invention provides antibodies that immunospecifically bind to an F protein of a mammalian metapneumovirus, said antibodies comprising a VL domain having an amino acid sequence of the VL domain of mAb234 (SEQ ID NO:18) or mAb338 (SEQ ID NO:26). The present invention provides antibodies that immunospecifically bind to an F protein of a mammalian metapneumovirus, said antibodies comprising a VL domain having an amino acid sequence that is at least 85%, 90%, 95%, 98%, 99% or at least 99.5% identical to the amino acid sequence of the VL domain of mAb234 (SEQ ID NO:18) or mAb338 (SEQ ID NO:26).

The present invention also provides antibodies that immunospecifically bind to an F protein of a mammalian metapneumovirus, said antibodies comprising a VL CDR having an amino acid sequence of any one of the VL CDRs listed in Table 1, infra. The present invention also provides antibodies that immunospecifically bind to an F protein of a mammalian metapneumovirus, said antibodies comprising a VL CDR having an amino acid sequence that is at least 85%, 90%, 95%, 98%, 99% or at least 99.5% identical to the amino acid sequence of any one of the VL CDRs listed in Table 1, infra.

In particular, the invention provides antibodies that immunospecifically bind to an F protein of a mammalian metapneumovirus, said antibodies comprising (or alternatively, consisting of) one, two, three or more VL CDRs having an amino acid sequence of any of the VL CDRs listed in Table 1, infra. In one embodiment, an antibody that immunospecifically binds to an F protein of a mammalian metapneumovirus comprises a VL CDR1 having the amino acid sequence of SEQ ID NO.: 20 or SEQ ID NO.: 28. In another embodiment, an antibody that immunospecifically binds to an F protein of a mammalian metapneumovirus comprises a VL CDR2 having the amino acid sequence of SEQ ID NO.: 22 or SEQ ID NO.: 30. In another embodiment, an antibody that immunospecifically binds to an F protein of a mammalian metapneumovirus comprises a VL CDR3 having the amino acid sequence of SEQ ID NO.: 24 or SEQ ID NO.: 32. In another embodiment, an antibody of that immunospecifically binds to an F protein of a mammalian metapneumovirus comprises a VL CDR1 having the amino acid sequence of SEQ ID NO.: 20 or SEQ ID NO.: 28 and a VL CDR2 having the amino acid sequence of SEQ ID NO.: 22 or SEQ ID NO.: 30. In another embodiment of an antibody that immunospecifically binds to an F protein of a mammalian metapneumovirus comprises a VL CDR1 having the amino acid sequence of SEQ ID NO.: 20 or SEQ ID NO.: 28 and a VL CDR3 having the amino acid sequence of SEQ ID NO.: 24 or SEQ ID NO.: 32. In another embodiment, an antibody that immunospecifically binds to an F protein of a mammalian metapneumovirus comprises a VL CDR2 having the amino acid sequence of SEQ ID NO.: 22 or SEQ ID NO.: 30 and a VL CDR3 having the amino acid sequence of SEQ ID NO.: 24 or SEQ ID NO.: 32. In another embodiment, an antibody that immunospecifically binds to an F protein of a mammalian metapneumovirus comprises a VL CDR1 having the amino acid sequence of SEQ ID NO.: 20 or SEQ ID NO.: 28, a VL CDR2 having the amino acid sequence of SEQ ID NO.: 22 or SEQ ID NO.: 30, and a VL CDR3 having the amino acid sequence of SEQ ID NO.: 24 or SEQ ID NO.:32, being a part of the antibody.

In certain embodiments, the invention provides an antibody that binds immunospecifically to the F protein of a mammalian metapneumovirus wherein the antibody comprises the amino acid sequence of one or more CDRs of mAb234 or mAb338 (i.e., the amino acid sequence of SEQ ID NO:4, 6, 8, 12, 14, 16, 20, 22, 24, 28, 30, or 32) or at least one amino acid sequence that has 1, 2, 3,4, 5, 6, or 7 amino acid substitutions, amino acid deletions, or amino acid additions relative to the amino acid sequence of a CDR of mAb234 or mAb338 (i.e., the amino acid sequence of SEQ ID NO:4, 6, 8, 12, 14, 16, 20, 22, 24, 28, 30, or 32). In a more specific embodiment, the amino acid substitution is a conservative amino acid substitution. In certain embodiments, the amino acid substitution is such that one amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). In certain embodiments, the amino acid substitution(s) is(are) at the amino acid position(s) that is(are) indicated in bold font in the sequences in Table 1.

TABLE 1

Residues that are different between each amino acid
sequence encoding the various CDRs appear in bold font.

| | | | | |
|---|---|---|---|---|
| Antibody Name | VH Domain | VH CDR1 | VH CDR2 | VH CDR3 |

| | | | | |
|---|---|---|---|---|
| mAb234 | SEQ ID NO:2 | FSLTDYGVS | VIWGDGNTNYHSALIS | SFGVYAMDY |
| | | SEQ ID NO:4 | SEQ ID NO:6 | SEQ ID NO:8 |
| encoded by: | SEQ ID NO:1 | SEQ ID NO:3 | SEQ ID NO:5 | SEQ ID NO:7 |
| mAb338 | SEQ ID NO:10 | FSLSSYGVH | VMWGDGSTNYHSGLIS | SFGVYAVDY |
| | | SEQ ID NO:12 | SEQ ID NO:14 | SEQ ID NO:16 |
| encodedby: | SEQ ID NO:9 | SEQ ID NO:11 | SEQ ID NO:13 | SEQ ID NO:15 |

| | | | | |
|---|---|---|---|---|
| Antibody name | VL Domain | VL CDR1 | VL CDR2 | VL CDR3 |

| | | | | |
|---|---|---|---|---|
| mAb234 | SEQ ID NO:18 | RTSQDTNNYIN | YTSMLHS | QQGDTLPPT |
| | | SEQ ID NO:20 | SEQ ID NO:22 | SEQ ID NO:24 |
| encoded by: | SEQ ID NO:17 | SEQ ID NO:19 | SEQ ID NO:21 | SEQ ID NO:23 |
| mAb338 | SEQ ID NO:26 | RASQDVNNYLN | YTSMLHS | QQGETLPPT |
| | | SEQ ID NO:28 | SEQ ID NO:30 | SEQ ID NO:32 |
| encoded by: | SEQ ID NO:25 | SEQ ID NO:27 | SEQ ID NO:29 | SEQ ID NO:31 |

The present invention provides antibodies that immunospecifically bind to an F protein of a mammalian metapneumovirus, such as a human metapneumovirus, said antibodies comprising a VH domain of mAb234 or mAb338 (i.e., SEQ ID NO:2 or 10) or a homolog thereof combined with a VL domain of mAb234 or mAb338 (i.e., SEQ ID NO:18 or 26) or a homolog thereof. Said antibodies may further comprise one or more CDRs of mAb234 or mAb338 (i.e., the amino acid sequence of SEQ ID NO:4, 6, 8, 12, 14, 16,20, 22,24, 28, 30, or 32) or at least one amino acid sequence that has 1, 2, 3, 4, 5, 6, or 7 amino acid substitutions, amino acid deletions, or amino acid additions relative to the amino acid sequence of a CDR of mAb234 or mAb338 (i.e., the amino acid sequence of SEQ ID NO:4, 6, 8, 12, 14, 16, 20, 22, 24, 28, 30, or 32).

The present invention provides antibodies that immunospecifically bind to an F protein of a mammalian metapneumovirus, said antibodies comprising one or more VH CDRs and one or more VL CDRs listed in Table 1, supra. In particular, the invention provides an antibody that immunospecifically binds to an F protein of a mammalian metapneumovirus, said antibody comprising (or alternatively, consisting of) a VH CDR1 and a VL CDR1; a VH CDR1 and a VL CDR2; a VH CDR1 and a VL CDR3; a VH CDR2 and a VL CDR1; VH CDR2 and VL CDR2; a VH CDR2 and a VL CDR3; a VH CDR3 and a VH CDR1; a VH CDR3 and a VL CDR2; a VH CDR3 and a VL CDR3; a VH1 CDR1, a VH CDR2 and a VL CDR1; a VH CDR1, a VH CDR2 and a VL CDR2; a VH CDR1, a VH CDR2 and a VL CDR3; a VH CDR2, a VH CDR3 and a VL CDR1, a VH CDR2, a VH CDR3 and a VL CDR2; a VH CDR2, a VH CDR2 and a VL CDR3; a VH CDR1, a VL CDR1 and a VL CDR2; a VH CDR1, a VL CDR1 and a VL CDR3; a VH CDR2, a VL CDR1 and a VL CDR2; a VH CDR2, a VL CDR1 and a VL CDR3; a VH CDR3, a VL CDR1 and a VL CDR2; a VH CDR3, a VL CDR1 and a VL CDR3; a VH CDR1, a VH CDR2, a VH CDR3 and a VL CDR1; a VH CDR1, a VH CDR2, a VH CDR3 and a VL CDR2; a VH CDR1, a VH CDR2, a VH CDR3 and a VL CDR3; a VH CDR1, a VH CDR2, a VL CDR1 and a VL CDR2; a VH CDR1, a VH CDR2, a VL CDR1 and a VL CDR3; a VH CDR1, a VH CDR3, a VL CDR1 and a VL CDR2; a VH CDR1, a VH CDR3, a VL CDR1 and a VL CDR3; a VH CDR2, a VH CDR3, a VL CDR1 and a VL CDR2; a VH CDR2, a VH CDR3, a VL CDR1 and a VL CDR3; a VH CDR2, a VH CDR3, a VL CDR2 and a VL CDR3; a VH CDR1, a VH CDR2, a VH CDR3, a VL CDR1 and a VL CDR2; a VH CDR1, a VH CDR2, a VH CDR3, a VL CDR1 and a VL CDR3; a VH CDR1, a VH CDR2, a VL CDR1, a VL CDR2, and a VL CDR3; a VH CDR1, a VH CDR3, a VL CDR1, a VL CDR2, and a VL CDR3; a VH CDR2, a VH CDR3, a VL CDR1, a VL CDR2, and a VL CDR3; or any combination thereof of the VH CDRs and VL CDRs listed in Table 1, supra.

In one embodiment, an antibody that immunospecifically binds to an F protein of a mammalian metapneumovirus comprises a VH CDR1 having the amino acid sequence of SEQ ID NO.: 4 or SEQ ID NO.: 12 and a VL CDR1 having the amino acid sequence of SEQ ID NO.: 20 or SEQ ID NO.: 28. In another embodiment, an antibody that immunospecifically binds to an F protein of a mammalian metapneumovirus comprises a VH CDR1 having the amino acid sequence of SEQ ID NO.: 4 or SEQ ID NO.: 12 and a VL CDR2 having the amino acid sequence of SEQ ID NO.: 22 or SEQ ID NO.: 30. In another embodiment, an antibody that immunospecifically binds to an F protein of a mammalian metapneumovirus comprises a VH CDR1 having the amino acid sequence of SEQ ID NO.: 4 or SEQ ID NO.: 12 and a VL CDR3 having an amino acid sequence of SEQ ID NO.: 24 or SEQ ID NO.: 32.

In one embodiment, an antibody that immunospecifically binds to an F protein of a mammalian metapneumovirus comprises a VH CDR2 having the amino acid sequence of SEQ ID NO.: 6 or SEQ ID NO.: 14 and a VL CDR1 having the amino acid sequence of SEQ ID NO.: 20 or SEQ ID NO.: 28. In another embodiment, an antibody that immunospecifically binds to an F protein of a mammalian metapneumovirus comprises a VH CDR2 having the amino acid sequence of SEQ ID NO.: 6 or SEQ ID NO.: 14 and a VL CDR2 having the amino acid sequence of SEQ ID NO.: 22 or SEQ ID NO.: 30. In another embodiment, an antibody that immunospecifically binds to an F protein of a mammalian metapneumovirus comprises a VH CDR2 having the amino acid sequence of SEQ ID NO.: 6 or SEQ ID NO.: 14 and a VL CDR3 having an amino acid sequence of SEQ ID NO.: 24 or SEQ D NO.: 32.

In one embodiment, an antibody that immunospecifically binds to an F protein of a mammalian metapneumovirus comprises a VH CDR3 having the amino acid sequence of SEQ ID NO.: 8 or SEQ ID NO.: 16 and a VL CDR1 having the amino acid sequence of SEQ ID NO.: 20 or SEQ ID NO.: 28. In another embodiment, an antibody that immunospecifically binds to an F protein of a mammalian metapneumovirus comprises a VH CDR3 having the amino acid sequence of SEQ ID NO.: 8 or SEQ ID NO.: 16 and a VL CDR2 having the amino acid sequence of SEQ ID NO.: 22 or SEQ ID NO.: 30. In another embodiment, an antibody that immunospecifically binds to an F protein of a mammalian metapneumovirus comprises a VH CDR3 having the amino acid sequence of SEQ ID NO.: 8 or SEQ ID NO.: 16 and a VL CDR3 having an amino acid sequence of SEQ ID NO.: 24 or SEQ ID NO.: 32.

The present invention provides for a nucleic acid molecule, generally isolated, encoding an antibody of the present invention (as described above) that immunospecifically binds to an F protein of a mammalian metapneumovirus. In particular, the invention provides an isolated nucleic acid molecule encoding an antibody of the invention wherein the nucleic acid comprises one or more of the following nucleotide sequences: SEQ ID NO:1, 3, 5, 7, 9, 11, 13, 15, 17, 19, 21, 23, 25, 27, 29, and/or 31 or a nucleotide sequence that is at least 50%, 60%, 70%, 80%, 85%, 90%, 95%, 98%, 99%, or 99.5% identical to SEQ ID NO:1, 3, 5, 7, 9, 11, 13, 15, 17, 19, 21, 23, 25, 27, 29, and/or 31.

In a specific embodiment, the invention provides a nucleic acid encoding mAb234 or mAb338.

The present invention provides nucleic acid molecules encoding antibodies that immunospecifically bind to an F protein of a mammalian metapneumovirus, said antibodies comprising one or more VH CDRs and one or more VL CDRs listed in Table 1, supra. In particular, the invention provides an isolated nucleic acid molecule encoding an antibody that immunospecifically binds to an F protein of a mammalian metapneumovirus, said antibody comprising (or alternatively, consisting of) a VH CDR1 and a VL CDR1; a VH CDR1 and a VL CDR2; a VH CDR1 and a VL CDR3; a VH CDR2 and a VL CDR1; VH CDR2 and VL CDR2; a VH CDR2 and a VL CDR3; a VH CDR3 and a VH CDR1; a VH CDR3 and a VL CDR2; a VH CDR3 and a VL CDR3; a VH1 CDR1, a VH CDR2 and a VL CDR1; a VH CDR1, a VH CDR2 and a VL CDR2; a VH CDR1, a VH CDR2 and a VL CDR3; a VH CDR2, a VH CDR3 and a VL CDR1, a VH CDR2, a VH CDR3 and a VL CDR2; a VH CDR2, a VH CDR2 and a VL CDR3; a VH CDR1, a VL CDR1 and a VL CDR2; a VH CDR1, a VL CDR1 and a VL CDR3; a VH CDR2, a VL CDR1 and a VL CDR2; a VH CDR2, a VL CDR1 and a VL CDR3; a VH CDR3, a VL CDR1 and a VL CDR2; a VH CDR3, a VL CDR1 and a VL CDR3; a VH CDR1, a VH CDR2, a VH CDR3 and a VL CDR1; a VH CDR1, a VH CDR2, a VH CDR3 and a VL CDR2; a VH CDR1, a VH CDR2, a VH CDR3 and a VL CDR3; a VH CDR1, a VH CDR2, a VL CDR1 and a VL CDR2; a VH CDR1, a VH CDR2, a VL CDR1 and a VL CDR3; a VH CDR1, a VH CDR3, a VL CDR1 and a VL CDR2; a VH CDR1, a VH CDR3, a VL CDR1 and a VL CDR3; a VH CDR2, a VH CDR3, a VL CDR1 and a VL CDR2; a VH CDR2, a VH CDR3, a VL CDR1 and a VL CDR3; a VH CDR2, a VH CDR3, a VL CDR2 and a VL CDR3; a VH CDR1, a VH CDR2, a VH CDR3, a VL CDR1 and a VL CDR2; a VH CDR1, a VH CDR2, a VH CDR3, a VL CDR1 and a VL CDR3; a VH CDR1, a VH CDR2, a VL CDR1, a VL CDR2, and a VL CDR3; a VH CDR1, a VH CDR3, a VL CDR1, a VL CDR2, and a VL CDR3; a VH CDR2, a VH CDR3, a VL CDR1, a VL CDR2, and a VL CDR3; or any combination thereof of the VH CDRs and VL CDRs listed in Table 1, supra. The sequence identified numbers of the nucleotide sequences encoding the different domains are also listed in Table 1.

In a specific embodiment, an antibody that immunospecifically binds to an F protein of a mammalian metapneumovirus comprises an amino acid sequence that is encoded by a nucleotide sequence that hybridizes to the nucleotide sequence of SEQ ID NO:1, 3, 5, 7, 9, 11, 13, 15, 17, 19, 21, 23, 25, 27, 29, and/or 31. Thus, the invention provides an antibody comprising a combination of domains of mAb234 or mAb338 listed in Table 1 as described above and antibodies comprising a combination of domains of mAb234 or mAb338 listed in Table 1, wherein one or more of the domains is encoded by a nucleic acid that hybridizes under stringent conditions to SEQ ID NO:1, 3, 5, 7, 9, 11, 13, 15, 17, 19, 21, 23, 25, 27, 29, or 31.

In another embodiment, an antibody that immunospecifically binds to an F protein of a mammalian metapneumovirus comprises an amino acid sequence of a VH domain or an amino acid sequence a VL domain encoded by a nucleotide sequence that hybridizes to the nucleotide sequence encoding the VH or VL domains of mAb234 or mAb338 under stringent conditions. In another embodiment, an antibody that immunospecifically binds to an F protein of a mammalian metapneumovirus comprises an amino acid sequence of a VH domain and an amino acid sequence of a VL domain encoded by a nucleotide sequence that hybridizes to the nucleotide sequence encoding the VH and VL domains of mAb234 or mAb338 under stringent conditions. In another embodiment, an antibody that immunospecifically binds to an F protein of a mammalian metapneumovirus comprises an amino acid sequence of a VH CDR or an amino acid sequence of a VL CDR encoded by a nucleotide sequence that hybridizes to the nucleotide sequence encoding any one of the VH CDRs or VL CDRs listed in Table 1 under stringent conditions. In another embodiment, an antibody that immunospecifically binds to a an F protein of a mammalian metapneumovirus comprises an amino acid sequence of a VH CDR and an amino acid sequence of a VL CDR encoded by nucleotide sequences that hybridize to the nucleotide sequences encoding any one of the VH CDRs listed in Table 1 and any one of the VL CDRs listed Table 1 under stringent conditions.

In another embodiment, the present invention provides an antibody that immunospecifically binds to an F protein of a mammalian metapneumovirus, said antibody comprising a VH domain and/or VL domain encoded by a nucleotide sequence that hybridizes to the nucleotide sequence encoding the VH domain and/or VL domain of mAb234 or mAb338 (SEQ ID NO.: 1 and/or 17 or SEQ ID NO.: 9 and/or 25, respectively) under stringent conditions. In another embodiment, the present invention provides an antibody that immunospecifically binds to an F protein of a mammalian metapneumovirus, said antibody comprising a VH CDR and/or VL CDR encoded by a nucleotide sequence that hybridizes to the nucleotide sequence of the VH CDR and/or VL CDR of 7 mAb234 or mAb338 under stringent conditions.

The present invention provides antibodies that immunospecifically bind to an F protein of a mammalian metapneumovirus, said antibodies comprising derivatives of the VH domains, VH CDRs, VL domains, or VL CDRs described herein that immunospecifically bind to an F protein of a mammalian metapneumovirus. Standard techniques known to those of skill in the art can be used to introduce mutations (e.g., deletions, additions, and/or substitutions) in the nucleotide sequence encoding an antibody of the invention, including, for example, site directed mutagenesis and PCR mediated mutagenesis which results in amino acid substitutions. Preferably, the derivatives include less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the original molecule. In a preferred embodiment, the derivatives have conservative amino acid substitutions are made at one or more predicted non essential amino acid residues (i.e., amino acid residues which are not critical for the antibody to immunospecifically bind to an F protein of a mammalian metapneumovirus). In certain embodiments, mutations can be introduced randomly along all or part of the coding sequence, such as by saturation mutagenesis. Following mutagenesis, the encoded antibody can be expressed and the ability of the antibody to bind to an F protein of a mammalian metapneumovirus can be determined. Any method known to the skilled artisan can be used to test the biological activity of the antibody. Such methods include, but are not limited to, direct testing for binding (e.g., Biacore), competitive binding with mAb234 or mAb338, or inhibition of growth of a mammalian metapneumovirus.

In a specific embodiment, an antibody that immunospecifically binds to an F protein of a mammalian metapneumovirus comprises an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of mAb234 or mAb338, or an antigen-binding fragment thereof. In another embodiment, an antibody that immunospecifically binds to an F protein of a mammalian metapneumovirus comprises an amino acid sequence of a VH domain that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the VH domain of mAb234 or mAb338. In another embodiment, an antibody that immunospecifically binds to an F protein of a mammalian metapneumovirus comprises an amino acid sequence of a VL domain that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the VL domain of mAb234 or mAb338.

In another embodiment, an antibody that immunospecifically binds to an F protein of a mammalian metapneumovirus comprises an amino acid sequence of one or more VL CDRs that are at least 35%, at least 40%, at least 45%, at least 50%, at least 55%, at least 60%, at least 65%, at least 70%, at least 75%, at least 80%, at least 85%, at least 90%, at least 95%, or at least 99% identical to any of the VL CDRs listed in Table 1. In another embodiment, an antibody that immunospecifically binds to an F protein of a mammalian metapneumovirus comprises an amino acid sequence of one or more VL CDRs that are at least 35%, at least 40%, at least 45%, at least 50%, at least 55%, at least 60%, at least 65%, at least 70%, at least 75%, at least 80%, at least 85%, at least 90%, at least 95%, or at least 99% identical to any of one of the VL CDRs listed in Table 1.

In another embodiment, the invention provides an antibody that immunospecifically binds to an F protein of a mammalian metapneumovirus, said antibody encoded by a nucleotide sequence that is at least 65%, preferably at least 70%, at least 75%, at least 80%, at least 85%, at least 90%, at least 95%, or at least 99% identical to the nucleotide sequence encoding mAb234 or mAb338. In another embodiment, the invention provides an antibody that immunospecifically binds to an F protein of a mammalian metapneumovirus, said antibody comprising a VH domain and/or VL domain encoded by a nucleotide sequence that is at least 65%, preferably at least 70%, at least 75%, at least 80%, at least 85%, at least 90%, at least 95%, or at least 99% identical to the nucleotide sequence of the VH domain and/or VL domain of mAb234 or mAb338 (see Table 1). In another embodiment, the invention provides an antibody that immunospecifically binds to an F protein of a mammalian metapneumovirus, said antibody comprising a VH CDR and/or a VL CDR encoded by a nucleotide sequence that is at last 65%, preferably at least 70%, at least 75%, at least 80%, at least 85%, at least 90%, at least 95%, or at least 99% identical to the nucleotide sequence of the VH CDR and/or VL CDR of mAb234 or mAb338.

The present invention encompasses antibodies that compete with an antibody described herein for binding to an F protein of a mammalian metapneumovirus. In particular, the present invention encompasses antibodies that compete with mAb234 or mAb338 or an antigen-binding fragment thereof for binding to the F protein of a mammalian metapneumovirus. In a specific embodiment, the invention encompasses an antibody that reduces the binding of mAb234 or mAb338 to an F protein of a mammalian metapneumovirus by at least 25%, at least 30%, at least 35%, at least 40 %, at least 45%, at least 50%, at least 55%, at least 60%, at least 65%, at least 70%, at least 75%, at least 80%, at least 85%, at least 90%, at least 95% or more, 25% to 50%, 45 to 75%, or 75 to 99% relative to a control in the competition assay described herein or competition assays well known in the art. In another embodiment, the invention encompasses an antibody that reduces binding of mAb234 or mAb338 to an F protein of a mammalian metapneumovirus by at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or more, or 25% to 50%, 45 to 75%, or 75 to 99% relative to a control in an ELISA competition assay.

In a specific embodiment, an ELISA competition assay may be performed in the following manner: recombinant F protein of a mammalian metapneumovirus is prepared in PBS at a concentration of 1 μg/ml. 100 μl of this solution is added to each well of an ELISA 98-well microtiter plate and incubated overnight at 4-8° C. The ELISA plate is washed with PBS supplemented with 0.1% Tween to remove excess recombinant F protein. Non-specific protein-protein interactions are blocked by adding 100 μl of bovine serum albumin (BSA) prepared in PBS to a final concentration of 1%. After one hour at room temperature, the ELISA plate is washed. Unlabeled competing antibodies are prepared in blocking solution at different concentrations. The concentration of the competing unlabeled antibodies may range from 10 μg/ml to 0.01 μg/ml. Control wells contain either blocking solution only or control antibodies at concentrations ranging from 1 μg/ml to 0.01 μg/ml. Test antibody (e.g., mAb234 or mAb338) labeled with horseradish peroxidase or biotin is added to competing antibody dilutions at a fixed final concentration of 1 μg/ml. The concentration of test antibody can be lowered to increase the sensitivity of the assay, e.g., the concentration of the test antibody can be 500 ng/ml, 100 ng/ml, 50 ng/ml, 10 ng/ml or 1 ng/ml. 100 μl of test and competing antibody mixtures are added to the ELISA wells in triplicate and the plate is incubated for 1 hour at room temperature. Residual unbound antibody is washed away. Bound test antibody is detected by adding 100 μl of horseradish peroxidase substrate to each well. The plate is incubated for 30 min. at room temperature, and absorbance is read using an automated plate reader. The average of triplicate wells is calculated. Antibodies which compete well with the test antibody reduce the measured absorbance compared with control wells.

In another embodiment, the invention encompasses an antibody that reduces the binding of an antibody comprising (alternatively, consisting of) an antigen-binding fragment (e.g., a VH domain, a VH CDR, a VL domain or a VL CDR) of mAb234 or mAb338 to an F protein of a mammalian metapneumovirus by at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 25% to 50%, 45 to 75%, or 75 to 99% relative to a control in a competition assay described herein or well-known to one of skill in the art, e.g., in an ELISA competition assay.

The present invention encompasses polypeptides or proteins comprising (alternatively, consisting of) VH domains that compete with the VH domain of mAb234 or mAb338 for binding to an F protein of a mammalian metapneumovirus. The present invention also encompasses polypeptides or proteins comprising (alternatively, consisting of) VL domains that compete with a VL domain of mAb234 or mAb338 for binding to an F protein of a mammalian metapneumovirus.

The present invention encompasses polypeptides or proteins comprising (alternatively, consisting of) VH CDRs that compete with a VH CDR listed in Table 1, supra, for binding to an F protein of a mammalian metapneumovirus. The present invention also encompasses polypeptides or proteins comprising (alternatively, consisting of) VL CDRs that compete with a VL CDR listed in Table 1, supra for binding to an F protein of a mammalian metapneumovirus.

In certain embodiments, the invention provides an antibody that immunospecifically bind to an F protein of a mammalian metapneumovirus as described above, wherein the antibody is modified, e.g., by the covalent attachment of any type of molecule to the antibody. For example, but not by way of limitation, the antibody can be modified by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formylation, metabolic synthesis of tunicamycin, etc. Additionally, an antibody of the invention may be modified to contain one or more non-classical amino acids.

The present invention also provides antibodies that immunospecifically bind to an F protein of a mammalian metapneumovirus, said antibodies comprising a framework region known to those of skill in the art (e.g., a human or non-human framework). The framework regions may be naturally occurring or consensus framework regions. Preferably, the fragment region of an antibody of the invention is human (see, e.g., Chothia et al., 1998, J. Mol. Biol. 278:457-479 for a listing of human framework regions, which is incorporated herein by reference in its entirety).

In certain embodiments, an antibody of the invention is a humanized antibody. Any method know to the skilled artisan to humanize an antibody may be used. In specific embodiments, an antibody of the invention is humanized using the methods taught in U.S. patent application Ser. No. 10/923,068 filed Aug. 20, 2004 (published as US 2005/0042664 on Feb. 24, 2005), which is incorporated herein in its entirety.

In certain embodiments, an antibody of the invention is a fully human antibody.

Completely human antibodies are particularly desirable for therapeutic treatment of human subjects. Human antibodies can be made by a variety of methods known in the art including phage display methods described above using antibody libraries derived from human immunoglobulin sequences. See also U.S. Pat. Nos. 4,444,887 and 4,716,111; and International Publication Nos. WO 98/46645, WO 98/50433, WO 98/24893, WO98/16654, WO 96/34096, WO 96/33735, and WO 91/10741; each of which is incorporated herein by reference in its entirety.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., PCT publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598, which are incorporated by reference herein in their entirety. In addition, companies such as Abgenix, Inc. (Freemont, Calif.) and Genpharm (San Jose, Calif.) can be engaged to provide human antibodies directed against a selected antigen using technology similar to that described above.

In certain embodiments, the invention provides an antibody that immunospecifically binds to an F protein of a mammalian metapneumovirus as described above, wherein the constant regions and/or the framework regions are from a species to which the antibody is to be administered, e.g., human, primate, avian (e.g., turkey or chicken), horse, goat, or bovine.

The present invention encompasses antibodies that immunospecifically bind to an F protein of a mammalian metapneumovirus, said antibodies comprising the amino acid sequence of mAb234 or mAb338 with mutations (e.g., one or more amino acid substitutions) in the framework regions. In certain embodiments, antibodies that immunospecifically bind to an F protein of a mammalian metapneumovirus comprise the amino acid sequence of mAb234 or mAb338 with one or more amino acid residue substitutions in the framework regions of the VH and/or VL domains. In certain embodiments, a humanized antibody of the invention is further modified to comprise one or more mutations, such as amino acid substitutions, in its framework.

In one embodiment, an antibody that immunospecifically binds to an F protein of a mammalian metapneumovirus inhibits and/or reduces the interaction between the F protein and a host cell by approximately 25%, approximately 30%, approximately 35%, approximately 45%, approximately 50%, approximately 55%, approximately 60%, approximately 65%, approximately 70%, approximately 75%, approximately 80%, approximately 85%, approximately 90%, approximately 95%, or approximately 98% relative to a control such as PBS or a control IgG antibody in an in vivo and/or in vitro assay described herein or well-known to one of skill in the art (e.g., an immunoassay such as an ELISA). In one embodiment, an antibody that immunospecifically binds to an F protein of a mammalian metapneumovirus inhibits and/or reduces the interaction between the F protein and a host cell by at least 25%, 30%, 35%, 45%, 50%, 55%, 60%, 65%, 70%, 75%, 80%, 85%, 90%, 95%, or at least 98% relative to a control such as PBS or a control IgG antibody in an in vivo and/or in vitro assay described herein or well-known to one of skill in the art (e.g., an immunoassay such as an ELISA). In one embodiment, an antibody that immunospecifically binds to an F protein of a mammalian metapneumovirus inhibits and/or reduces the interaction between the F protein and a host cell by at most 25%, 30%, 35%, 45%, 50%, 55%, 60%, 65%, 70%, 75%, 80%, 85%, 90%, 95%, or at most 98% relative to a control such as PBS or a control IgG antibody in an in vivo and/or in vitro assay described herein or well-known to one of skill in the art (e.g., an immunoassay such as an ELISA).

In certain embodiments, an antibody of the invention inhibits and/or reduces the ability of a mammalian metapneumovirus, such as a human metapneumovirus, to infect a host cell, such as a mammalian host cell, by at least 25%, preferably at least 30%, at least 35%, at least 40%, at least 50%, at least 55%, at least 60%, at least 65%, at least 70%, at least 75%, at least 80%, at least 85%, at least 90%, at least 95%, or at least 98% relative to a control such as PBS or a control IgG antibody in an in vivo and/or in vitro assay.

In a specific embodiment, an antibody that immunospecifically binds to an F protein of a mammalian metapneumovirus acts synergistically with an antiviral agent to inhibit or reduce an infection with a mammalian metapneumovirus.

The antibodies of the present invention that immunospecifically bind to an F protein of a mammalian metapneumovirus may be monospecific, bispecific, trispecific or of greater multispecificity. Multispecific antibodies may be specific for different epitopes of an F protein of a mammalian metapneumovirus or may be specific for both an F protein of a mammalian metapneumovirus as well as for an epitope of another protein of the mammalian metapneumovirus. See, e.g., International publications WO 93/17715, WO 92/08802, WO 91/00360, and WO 92/05793; Tutt, et al., J. Immunol. 147:60-69(1991); U.S. Pat. Nos. 4,474,893, 4,714,681, 4,925,648, 5,573,920, and 5,601,819; and Kostelny et al., J. Immunol. 148:1547-1553 (1992).

The present invention provides for antibodies that have a high binding affinity for an F protein of a mammalian metapneumovirus. In a specific embodiment, an antibody that immunospecifically binds to an F protein of a mammalian metapneumovirus polypeptide has an association rate constant or kₒₙ rate (antibody (Ab)+antigen (Ag)→Ab-Ag) of at least 10⁵ M⁻¹s⁻¹, at least 1.5×10⁵ M⁻¹s⁻¹, at least 2×10⁵ M⁻¹s⁻¹, at least 2.5×10⁵ M⁻¹s⁻¹, at least 5×10⁵ M⁻¹s⁻¹, at least 10⁶ M⁻¹s⁻¹, at least 5×10⁶ M⁻¹s⁻¹, at least 10⁷ M⁻¹s⁻¹, at least 5×10⁷ M⁻¹s⁻¹, or at least 10⁸ M⁻¹s⁻¹, or 10⁵-10⁸ M⁻¹s⁻¹, 1.5×10⁵ M⁻¹s⁻¹-1×10⁷ M⁻¹s⁻¹, 2×10⁵-1×10⁶ M⁻¹s⁻¹, or 4.5×10⁵×10⁷ M⁻¹s⁻¹. In a preferred embodiment, an antibody that immunospecifically binds to an F protein of a mammalian metapneumovirus has a kon of at least 2×10⁵ M⁻¹s⁻¹, at least 2.5×10⁵ M⁻¹s⁻¹, at least 5×10⁵ M⁻¹s⁻¹, at least 10⁶ M⁻¹s⁻¹, at least 5×10⁶ M⁻¹ s⁻¹, at least 10⁷ M⁻¹s⁻¹, at least 5×10⁷ M⁻¹s⁻¹, or at least 10⁸ M⁻¹s⁻¹ as determined by a BIAcore assay.

In certain embodiments, an antibody that immunospecifically binds to an F protein of a mammalian metapneumovirus has a kₒₙ of at most 10⁸ M⁻¹s⁻¹, at most 10⁹ M⁻¹s⁻¹, at most 10¹⁰ M⁻¹s⁻¹, at most 10¹¹ M⁻¹s⁻¹, or at most 10¹² M⁻¹s⁻¹ as determined by a BIAcore assay.

In another embodiment, an antibody that immunospecifically binds to an F protein of a mammalian metapneumovirus has a k_{off} rate (antibody (Ab)+antigen (Ag)→Ab-Ag) of less than 10⁻³ s⁻¹, less than 5×10⁻³ s⁻¹, less than 10⁻⁴ s⁻¹, less than 2×10⁻⁴ s⁻¹, less than 5×10⁻⁴ s⁻¹, less than 10⁻⁵ s⁻¹, less than 5×10⁻⁵ s⁻¹, less than 10⁻⁶ s⁻¹, less than 5×10⁻⁶ s⁻¹, less than 10⁻⁷ s⁻¹, less than 5×10⁻⁷ s⁻¹, less than 10⁻⁸ s⁻¹, less than 5×10⁻⁸ s⁻¹, less than 5×10⁻⁹ s⁻¹, less than 5×10⁻⁹ s⁻¹, or less than 10⁻¹⁰ s⁻¹, or 10⁻³-10⁻¹⁰ s⁻¹, 10⁻⁴-10⁻⁸ s⁻¹, or 10⁻⁵-10⁻⁸ s⁻¹. In another embodiment an antibody that immunospecifically binds to an F protein of a mammalian metapneumovirus has a k_{off} rate (antibody (Ab)+antigen (Ag)→Ab-Ag) of greater than 10⁻³ s⁻¹, less than 5×10⁻³ s⁻¹, less than 10⁻⁴ s⁻¹, less than 2×10⁻⁴ s⁻¹, less than 5×10⁻⁴ s⁻¹, less than 10⁻⁵ s⁻¹, less than 5×10⁻⁵ s⁻¹, less than 10⁻⁶ s⁻¹, less than 5×10⁻⁶ s⁻¹, less than 10⁻⁷ s⁻¹, less than 5×10⁻⁷ s⁻¹, less than 10⁻⁸ s⁻¹, less than 5×10⁻⁸ s⁻¹, less than 10⁻⁹ s⁻¹, less than 5×10⁻⁹ s⁻¹, or less than 10⁻¹⁰ s⁻¹, or 10⁻³-10⁻¹⁰ s⁻¹, 10⁻⁴-10⁻⁸ s⁻¹, or 10⁻⁵-10⁻⁸ s⁻¹.

In another embodiment, an antibody that immunospecifically binds to an F protein of a mammalian metapneumovirus has an affinity constant or Kₐ (kon/koff) of at least 10² M⁻¹, at least 5×10² M⁻¹, at least 10³ M⁻¹, at least 5×10³ M⁻¹, at least 10⁴ M⁻¹, at least 5×10⁴ M⁻¹, at least 10⁵ M⁻¹, at least 5×10⁵ M⁻¹, at least 10⁶ M⁻¹, at least 5×10⁶ M⁻¹, at least 10⁷ M⁻¹, at least 5×10⁷ M⁻¹, at least 10⁸ M⁻¹, at least 5×10⁸ M⁻¹, at least 10⁹ M⁻¹, at least 5×10⁹ M⁻¹, at least 10¹⁰ M⁻¹, at least 5×10¹⁰ M⁻¹, at least 10¹¹ M⁻¹, at least 5×10¹¹ M⁻¹, at least 10¹² M⁻¹, at least 5×10¹² M⁻¹, at least 10¹³ M⁻¹, at least 5×10¹³ M⁻¹, at least 10¹⁴ M⁻¹, at least 5×10¹⁴ M⁻¹, at least 10¹⁵ M⁻¹, or at least 5×10¹⁵ M⁻¹, or 10²-5×10⁵ M⁻¹, 10⁴-1×10¹⁰ M⁻¹, or 10⁵-1×10⁸ M⁻¹. In another embodiment, an antibody that immunospecifically binds to an F protein of a mammalian metapneumovirus has an affinity constant or Kₐ (kon/koff) of at most 10² M⁻¹, at most 5×10² M⁻¹, at most 10³ M⁻¹, at most 5×10³ M⁻¹, at most 10⁴ M⁻¹, at most 5×10⁴ M⁻¹, at most 10⁵ M⁻¹, at most 5×10⁵ M⁻¹, at most 10⁶ M⁻¹, at most 5×10⁶ M⁻¹, at most 10⁷ M⁻¹, at most 5×10⁷ M⁻¹, at most 10⁸ M⁻¹, at most 5×10⁸ M⁻¹, at most 10⁹ M⁻¹, at most 5×10⁹ M⁻¹, at most 10¹⁰ M⁻¹, at most 5×10¹⁰ M⁻¹, at most 10¹¹ M⁻¹, at most 5×10¹¹ M⁻¹, at most 10¹² M⁻¹, at most 5×10¹² M⁻¹, at most 10¹³ M⁻¹, at most 5×10¹³ M⁻¹, at most 10¹⁴ M⁻¹, at most 5×10¹⁴ M⁻¹, at most 10¹⁵ M⁻¹, or at most 5×10⁵ M⁻¹.

In another embodiment, an antibody that immunospecifically binds to an F protein of a mammalian metapneumovirus has a dissociation constant or Kd (k_{off}/kₒₙ) of less than 10⁻⁵ M, less than 5×10⁻⁵ M, less than 10⁻⁶ M, less than 5×10⁻⁶ M, less than 10⁻⁷ M, less than 5×10⁻⁷ M, less than 10⁻⁸ M, less than 5×10⁻⁸ M, less than 10⁻⁹ M, less than 5×10⁻⁹ M, less than 10⁻¹⁰ M, less than 5×10⁻¹⁰ M, less than 10⁻¹¹ M, less than 5×10¹¹ M, less than 10⁻¹² M, less 5×10⁻¹² M, less than 10⁻¹³ M, less than 5×10⁻¹³ M, less than 10⁻¹⁴ M, less than 5×10⁻¹⁴ M, less than 10⁻¹⁵ M, or less than 5×10⁻¹⁵ M or 10⁻² M-5×10⁻⁵ M, 10⁻⁶-10⁻¹⁵ M, or 10⁻⁸-10⁻¹⁴ M. In another embodiment, an antibody that immunospecifically binds to an F protein of a mammalian metapneumovirus has a dissociation constant or Kd (k_{off}/kₒₙ) of greater than 10⁻⁵ M, greater than 5×10⁻⁵ M, greater than 10⁻⁶ M, greater than 5×10⁻⁶ M, greater than 10⁻⁷ M, greater than 5×10⁻⁷ M, greater than 10⁻⁸ M, greater than 5×10⁻⁸ M, greater than 10⁻⁹ M, greater than 5×10⁻⁹ M, greater than 10⁻¹⁰ M, greater than 5×10⁻¹⁰ M, greater than 10⁻¹¹ M, greater than 5×10^{'11} M, greater than 10⁻¹² M, greater than 5×10⁻¹² M, greater than 10⁻¹³ M, greater than 5×10⁻¹³ M, greater than 10⁻¹⁴ M, greater than 5×10⁻¹⁴ M, greater than 10⁻¹⁵ M, or greater than 5×10⁻¹⁵ M.

The antibodies of the invention do not include antibodies known in the art that immunospecifically bind to an F protein of a mammalian metapneumovirus. However, the antibodies of the invention may include those that cross react with both an F protein of a mammalian metapneumovirus and an F protein of respiratory syncytial virus.

The present invention provides peptides, polypeptides and/or proteins comprising one or more variable or hypervariable regions of the antibodies described herein. Preferably, peptides, polypeptides or proteins comprising one or more variable or hypervariable regions of antibodies of the invention further comprise a heterologous amino acid sequence. In certain embodiments, such a heterologous amino acid sequence comprises at least 5 contiguous amino acid residues, at least 10 contiguous amino acid residues, at least 15 contiguous amino acid residues, at least 20 contiguous amino acid residues, at least 25 contiguous amino acid residues, at least 30 contiguous amino acid residues, at least 40 contiguous amino acid residues, at least 50 contiguous amino acid residues, at least 75 contiguous amino acid residues, at least 100 contiguous amino acid residues or more contiguous amino acid residues. Such peptides, polypeptides and/or proteins may be referred to as fusion proteins.

In a specific embodiment, peptides, polypeptides or proteins comprising one or more variable or hypervariable regions of the antibodies of the invention are 10 amino acid residues, 15 amino acid residues, 20 amino acid residues, 25 amino acid residues, 30 amino acid residues, 35 amino acid residues, 40 amino acid residues, 45 amino acid residues, 50 amino acid residues, 75 amino acid residues, 100 amino acid residues, 125 amino acid residues, 150 amino acid residues or more amino acid residues in length. In certain embodiments, peptides, polypeptides, or proteins comprising one or more variable or hypervariable regions of an antibody of the invention immunospecifically bind to an F protein of a mammalian metapneumovirus.

In a specific embodiment, the present invention provides peptides, polypeptides and/or proteins comprising a VH domain and/or VL domain of one of the antibodies described herein (see Table 1). In a preferred embodiment, the present invention provides peptides, polypeptides and/or proteins comprising one or more CDRs having the amino acid sequence of any of the CDRs listed in Table 1. In accordance with these embodiments, the peptides, polypeptides or proteins may further comprise a heterologous amino acid sequence.

Peptides, polypeptides or proteins comprising one or more variable or hypervariable regions have utility, e.g., in the production of anti-idiotypic antibodies. The anti-idiotypic antibodies produced can also be utilized in immunoassays, such as, e.g., ELISAs, for the detection of antibodies which comprise a variable or hypervariable region contained in the peptide, polypeptide or protein used in the production of the anti-idiotypic antibodies.

In certain specific embodiments, an antibody of the invention binds specifically to a subgroup of human metapneumovirus, i.e., subgroup A or subgroup B. In certain aspects, an antibody of the invention binds the F protein of a subgroup A human metapneumovirus with at least 5-fold, 10-fold, 50-fold, 100-fold, or 1000-fold higher affinity than the F protein of a subgroup B human metapneumovirus. In certain aspects, an antibody of the invention binds the F protein of a subgroup A human metapneumovirus with at most 5-fold, 10-fold, 50-fold, 100-fold, or 1000-fold higher affinity than the F protein of a subgroup B human metapneumovirus. In certain aspects, an antibody of the invention binds the F protein of a subgroup B human metapneumovirus with at least 5-fold, 10-fold, 50-fold, 100-fold, or 1000-fold higher affinity than the F protein of a subgroup A human metapneumovirus. In certain aspects, an antibody of the invention binds the F protein of a subgroup B human metapneumovirus with at most 5-fold, 10-fold, 50-fold, 100-fold, or 1000-fold higher affinity than the F protein of a subgroup A human metapneumovirus. In certain aspects, an antibody of the invention binds specifically to both subgroups A and B of human metapneumovirus with comparable affinity.

In certain specific embodiments, an antibody binds specifically to a subtype of human metapneumovirus, i.e., subtype A1, A2, B1, and B2. In certain aspects, an antibody of the invention binds the F protein of one subtype of human metapneumovirus with at least 5-fold, 10-fold, 50-fold, 100-fold, or 1000-fold higher affinity than the F protein of a different subtype of human metapneumovirus. In certain aspects, an antibody of the invention binds the F protein of one subtype of human metapneumovirus with at most 5-fold, 10-fold, 50-fold, 100-fold, or 1000-fold higher affinity than the F protein of a different subtype of human metapneumovirus.

In certain embodiments, the antibody of the invention binds to the F protein of all subtypes of mammalian metapneumovirus, i.e., subtype A1, A2, B1, and B2. In certain embodiments, an antibody binds specifically to a subtype of human metapneumovirus, i.e., subtype A1, A2, B1, and B2.

In certain embodiments, an antibody of the invention protects a mammal against infection with mammalian metapneumovirus. In certain, more specific embodiments, an antibody of the invention protects humans against infection with human metapneumovirus. In certain embodiments, an antibody of the invention protects birds (e.g., chickens, turkeys, and ducks) against infection with avian pneumovirus.

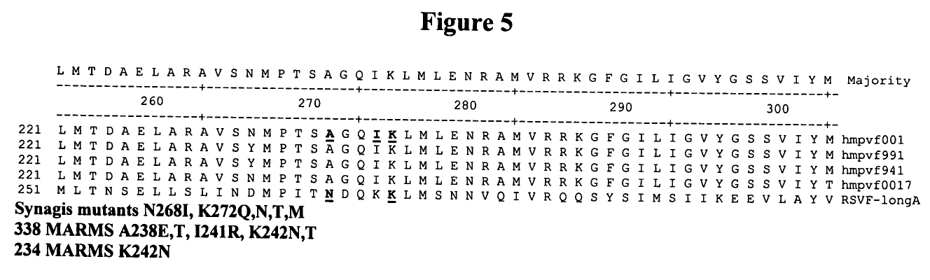

In certain embodiments, an antibody of the invention binds to an F protein of a mammalian metapneumovirus in the vicinity, i.e., within 50 amino acids, within 25 amino acids, within 10 amino acids, within 5 amino acids, or within 2 amino acids, of any one of the amino acid substitutions that were identified as conferring resistance to mAb338 or mAb234 (FIG. 5, section 6.2). In a specific embodiment, an antibody of the invention is an antibody against which a mutation that confers resistance to mAb338 or mAb234 also confers resistance.

In certain embodiments, the recitation of sequence identities refers to an alignment over the entire length of the nucleotide sequence or amino acid sequence of the respective SEQ ID NO.

5.1.1 Antibodies Having Increased Half-Lives

The present invention provides for antibodies that immunospecifically bind to an F protein of a mammalian metapneumovirus which have an extended half-life in vivo. In particular, the present invention provides antibodies that immunospecifically bind to an F protein of a mammalian metapneumovirus which have a half-life in a subject, preferably a mammal and most preferably a human, of greater than 3 days, greater than 7 days, greater than 10 days, preferably greater than 15 days, greater than 25 days, greater than 30 days, greater than 35 days, greater than 40 days, greater than 45 days, greater than 2 months, greater than 3 months, greater than 4 months, or greater than 5 months.

To prolong the serum circulation of antibodies (e.g., monoclonal antibodies, single chain antibodies and Fab fragments) in vivo, for example, inert polymer molecules such as high molecular weight polyethyleneglycol (PEG) can be attached to the antibodies with or without a multifunctional linker either through site-specific conjugation of the PEG to the N— or C-terminus of the antibodies or via epsilon-amino groups present on lysine residues. Linear or branched polymer derivatization that results in minimal loss of biological activity will be used. The degree of conjugation can be closely monitored by SDS-PAGE and mass spectrometry to ensure proper conjugation of PEG molecules to the antibodies. Unreacted PEG can be separated from antibody-PEG conjugates by size-exclusion or by ion-exchange chromatography. PEG-derivatized antibodies can be tested for binding activity as well as for in vivo efficacy using methods well-known to those of skill in the art, for example, by immunoassays described herein.

Antibodies having an increased half-life in vivo can also be generated introducing one or more amino acid modifications (i.e., substitutions, insertions or deletions) into an IgG constant domain, or FcRn binding fragment thereof (preferably a Fc or hinge Fc domain fragment). See, e.g., International Publication No. WO 98/23289; International Publication No. WO 97/34631; International Publication No. WO 02/060919; and U.S. Pat. No. 6,277,375, each of which is incorporated herein by reference in its entirety.

Further, antibodies can be conjugated to albumin in order to make the antibody or antibody fragment more stable in vivo or have a longer half life in vivo. The techniques are well-known in the art, see, e.g., International Publication Nos. WO 93/15199, WO 93/15200, and WO 01/77137; and European Patent No. EP 413,622, all of which are incorporated herein by reference.

5.1.2 Antibody Conjugates

The present invention provides an antibody or fragments thereof that immunospecifically binds to an F protein of a mammalian metapneumovirus wherein the antibody is recombinantly fused or chemically conjugated (including both covalent and non-covalent conjugations) to a heterologous protein or polypeptide (or fragment thereof, preferably to a polypeptide of at least 10, at least 20, at least 30, at least 40, at least 50, at least 60, at least 70, at least 80, at least 90 or at least 100 amino acids) to generate fusion proteins. In particular, the invention provides fusion proteins comprising an antigen-binding fragment of an antibody described herein (e.g., a Fab fragment, Fd fragment, Fv fragment, F(ab)2 fragment, a VH domain, a VH CDR, a VL domain or a VL CDR (see Table 1)) and a heterologous protein, polypeptide, or peptide. Preferably, the heterologous protein, polypeptide, or peptide that the antibody or antibody fragment is fused to is useful for targeting the antibody to tissue that is being infected with mammalian metapneumovirus or at risk of being infected with mammalian metapneumovirus. In a specific embodiment, an antibody that immunospecifically binds to an F protein of a mammalian metapneumovirus is fused or conjugated to an anti-viral agent. Methods for fusing or conjugating proteins, polypeptides, or peptides to an antibody or an antibody fragment are known in the art. See, e.g., U.S. Pat. Nos. 5,336,603, 5,622,929, 5,359,046, 5,349,053, 5,447,851, and 5,112,946; European Patent Nos. EP 307,434 and EP 367,166; International Publication Nos. WO 96/04388 and WO 91/06570; Ashkenazi et al., 1991, Proc. Natl. Acad. Sci. USA 88: 10535-10539; Zheng et al., 1995, J. Immunol. 154:5590-5600; and Vil et al., 1992, Proc. Natl. Acad. Sci. USA 89:11337-11341 (said references are incorporated herein by reference in their entireties).

Additional fusion proteins may be generated through the techniques of gene-shuffling, motif-shuffling, exon-shuffling, and/or codon-shuffling (collectively referred to as “DNA shuffling”). DNA shuffling may be employed to alter the activities of antibodies of the invention or fragments thereof (e.g., antibodies or fragments thereof with higher affinities and lower dissociation rates). See, generally, U.S. Pat. Nos. 5,605,793, 5,811,238, 5,830,721, 5,834,252, and 5,837,458; Patten et al., 1997, Curr. Opinion Biotechnol. 8:724-33; Harayama, 1998, Trends Biotechnol. 16(2):76-82; Hansson, et al., 1999, J. Mol. Biol. 287:265-76; and Lorenzo and Blasco, 1998, Biotechniques 24(2):308-313 (each of these patents and publications are hereby incorporated by reference in its entirety). Antibodies or fragments thereof, or the encoded antibodies or fragments thereof, may be altered by being subjected to random mutagenesis by error-prone PCR, random nucleotide insertion or other methods prior to recombination. A polynucleotide encoding an antibody or fragment thereof that immunospecifically binds to an F protein of a mammalian metapneumovirus may be recombined with one or more components, motifs, sections, parts, domains, fragments, etc. of one or more heterologous molecules.

Moreover, the antibodies or fragments thereof can be fused to marker sequences, such as a peptide to facilitate purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., 1989, Proc. Natl. Acad. Sci. USA 86:821-824, for instance, hexa-histidine provides for convenient purification of the fusion protein. Other peptide tags useful for purification include, but are not limited to, the hemagglutinin (“HA”) tag, which corresponds to an epitope derived from the influenza hemagglutinin protein (Wilson et al., 1984, Cell 37:767), and the “flag” tag.

In other embodiments, antibodies of the present invention or fragments thereof conjugated to a diagnostic or detectable agent. Such antibodies can be useful for monitoring or prognosing the onset, development, progression and/or severity of an infection with a mammalian metapneumovirus. Such diagnosis and detection can accomplished by coupling the antibody to detectable substances including, but not limited to, various enzymes, such as, but not limited to, horseradish peroxidase, alkaline phosphatase, beta-galactosidase, or acetylcholinesterase; prosthetic groups, such as, but not limited to, streptavidin/biotin and avidin/biotin; fluorescent materials, such as, but not limited to, umbelliferone, fluorescein, fluorescein isothiocynate, rhodamine, dichlorotriazinylamine fluorescein, dansyl chloride or phycoerythrin; luminescent materials, such as, but not limited to, luminol; bioluminescent materials, such as but not limited to, luciferase, luciferin, and aequorin; radioactive materials, such as, but not limited to, iodine (131I, 125I, 123I, and 121I,), carbon (14C), sulfur (35S), tritium (3H), indium (115In, 113In, 112In, and 111In,), technetium (99Tc), thallium (201Ti), gallium (68Ga, 67Ga), palladium (103Pd), molybdenum (99Mo), xenon (133Xe), fluorine (18F), 153Sm, 177Lu, 159Gd, 149Pm, 140La, 175Yb, 166Ho, 90Y, 47Sc, 186Re, 188Re, 142 Pr, 105Rh, 97Ru, 68Ge, 57Co, 65Zn, 85Sr, 32P, 153Gd, 169Yb, 51Cr, 54Mn, 75Se, 113Sn, and 117Sn; and positron emitting metals using various positron emission tomographies, and non-radioactive paramagnetic metal ions.

Techniques for conjugating therapeutic moieties to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies 84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., 1982, Immunol. Rev. 62:119-58.

Alternatively, an antibody of the invention can be conjugated to a second antibody to form an antibody heteroconjugate as described by Segal in U.S. Pat. No. 4,676,980, or an antibody heteropolymer as described by Taylor in U.S. Pat. No. 5,470,570, or as described by Mohamed et al in International Patent Application WO 2004/024889, all of which are incorporated herein by reference in its entirety.

Antibodies of the invention may also be attached to solid supports, which are particularly useful for immunoassays or purification of mammalian metapneumovirus or the F protein of mammalian metapneumovirus. Such solid supports include, but are not limited to, glass, cellulose, polyacrylamide, nylon, polystyrene, polyvinyl chloride or polypropylene.

5.1.3. Strategies for Generating MPV F Protein-Specific Antibodies

Any technique known to the skilled artisan can be used to generate antibodies that bind immunospecifically to an F protein of a mammalian metapneumovirus, such as human metapneumovirus. Such techniques include, but are not limited to standard hybridoma technology using mice, hamsters, or Hu-mAb-mice and recombinant technology. Immunization for the hybridoma techniques can be performed by, e.g., DNA immunization, infection with a chimeric virus expressing the F protein, immunization with transfected cells expressing the F protein, infection with mammalian metapneumovirus, immunization with MPV-infected cells, immunization with Adenovirus-vectored MPV F protein, and immunization with hMPV F protein. An illustrative recombinant technology is phage display (Dyax) using soluble MPV F as the target. Individual fragments or epitope of the F protein can also be used for immunization.

Monoclonal antibodies can be selected and isolated using any method known to the skilled artisan. In an illustrative embodiment, positive hybridomas are selected using an infected cell ELISA. Hybridomas with 5-fold over background cell reactivity are selected. Posititve hybridomas are expanded to 24 wells and retested using infected cell ELISA. In vitro neutralization is also tested at this stage. Subsequently, limited dilution cloning is performed. The hybridomas are retested using infected cell ELISA and neutralizing effect. The positive hyridomas can then be used to produce and purify the antibodies using any method known to the skilled artisan.

5.2 Therapies

The present invention also provides methods for preventing, managing, treating, and/or ameliorating infections with mammalian metapneumovirus. The present invention also provides compositions comprising one or more antibodies that immunospecifically bind to an F protein of a mammalian metapneumovirus and one or more prophylactic or therapeutic agents other than antibodies that immunospecifically bind to an F protein of a mammalian metapneumovirus and methods of preventing, managing, treating, and/or ameliorating a disease or disorder utilizing said compositions. Therapeutic or prophylactic agents include, but are not limited to, small molecules, synthetic drugs, peptides, polypeptides, proteins, nucleic acids (e.g., DNA and RNA nucleotides including, but not limited to, antisense nucleotide sequences, triple helices, RNAi, and nucleotide sequences encoding biologically active proteins, polypeptides or peptides) antibodies, synthetic or natural inorganic molecules, mimetic agents, and synthetic or natural organic molecules.

The antibodies of the present invention can be used in combination with other anti-viral agents against other viruses to provide a broadspectrum antiviral treatment and/or prevention. Such broadspectrum antiviral treatments are described, e.g., in U.S. application Ser. No. 10/628,088 filed Jul. 25, 2003 (published as US 2004/0096451 on May 20, 2004), which is incorporated herein by reference in its entirety.

In certain embodiments, one or more immunomodulatory agents are administered in combination with an antibody of the invention to treat or prevent an infection with mammalian metapneumovirus.

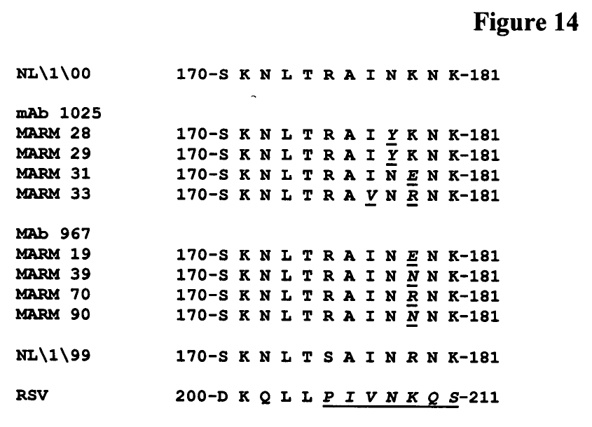

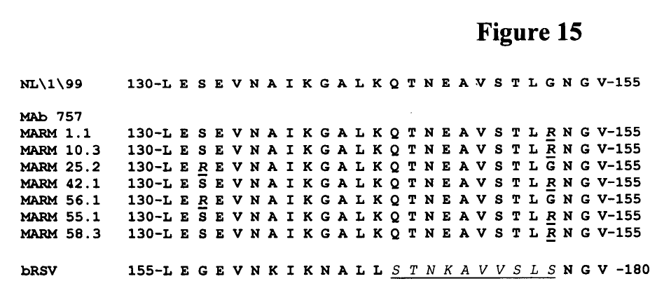

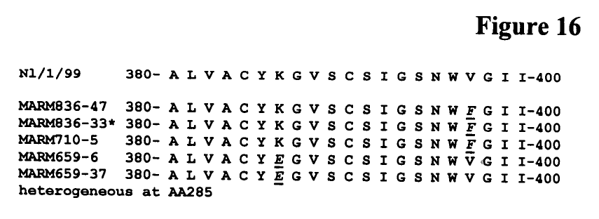

In certain embodiments of the invention, peptides comprising the MARMs identified herein (FIGS. 5, 13 to 16) can be administered to a subject. In more specific embodiments, the peptides are administered in combination with the antibodies that were used to select the MARM that is carried by the peptide. Without being bound by theory, the MARM bearing peptide will induce antibodies against any viruses that may evolve in the subject to evade the neutralizing effect of the antibody that is being administered. In certain embodiments, the peptide is at least 5, 10, 15, 25, 50, 75, 100, 250, or 500 amino acids in length. In certain embodiments, the peptide is at most 5, 10, 15, 25, 50, 75, 100, 250, or 500 amino acids in length.

5.2.1 Anti-Viral Agents

Any anti-viral agent well-known to one of skill in the art can be used in the compositions and the methods of the invention in addition to an antibody of the invention. Non-limiting examples of anti-viral agents include proteins, polypeptides, peptides, fusion proteins antibodies, nucleic acid molecules, organic molecules, inorganic molecules, and small molecules that inhibit and/or reduce the attachment of a virus to its receptor, the internalization of a virus into a cell, the replication of a virus, or release of virus from a cell. In particular, anti-viral agents include, but are not limited to, nucleoside analogs (e.g., zidovudine, acyclovir, gangcyclovir, vidarabine, idoxuridine, trifluridine, and ribavirin), foscarnet, amantadine, rimantadine, saquinavir, indinavir, ritonavir, alpha-interferons and other interferons, and AZT.

In specific embodiments, the anti-viral agent is an immunomodulatory agent that is immunospecific for a viral antigen. As used herein, the term “viral antigen” includes, but is not limited to, any viral peptide, polypeptide and protein (e.g., HIV gp120, HIV nef, RSV F glycoprotein, RSV G glycoprotein, influenza virus neuraminidase, influenza virus hemagglutinin, HTLV tax, herpes simplex virus glycoprotein (e.g., gB, gC, gD, and gE) and hepatitis B surface antigen, and a PIV antigen) that is capable of eliciting an immune response. Antibodies useful in this invention for treatment of a viral infectious disease include, but are not limited to, antibodies against antigens of pathogenic viruses, including as examples and not by limitation: adenovirdiae (e.g., mastadenovirus and aviadenovirus), herpesviridae (e.g., herpes simplex virus 1, herpes simplex virus 2, herpes simplex virus 5, and herpes simplex virus 6), leviviridae (e.g., levivirus, enterobacteria phase MS2, allolevirus), poxviridae (e.g., chordopoxvirinae, parapoxvirus, avipoxvirus, capripoxvirus, leporiipoxvirus, suipoxvirus, molluscipoxvirus, and entomopoxvirinae), papovaviridae (e.g., polyomavirus and papillomavirus), paramyxoviridae (e.g., paramyxovirus, parainfluenza virus 1, mobillivirus (e.g., measles virus), rubulavirus (e.g., mumps virus), pneumonovirinae (e.g., pneumovirus, human respiratory synctial virus), and avian pneumovirus), picomaviridae (e.g., enterovirus, rhinovirus, hepatovirus (e.g., human hepatits A virus), cardiovirus, and apthovirus), reoviridae (e.g., orthoreovirus, orbivirus, rotavirus, cypovirus, fijivirus, phytoreovirus, and oryzavirus), retroviridae (e.g., mammalian type B retroviruses, mammalian type C retroviruses, avian type C retroviruses, type D retrovirus group, BLV-HTLV retroviruses, lentivirus (e.g. human immunodeficiency virus 1 and human immunodeficiency virus 2), spumavirus), flaviviridae (e.g., hepatitis C virus), hepadnaviridae (e.g., hepatitis B virus), togaviridae (e.g., alphavirus (e.g., sindbis virus) and rubivirus (e.g., rubella virus)), rhabdoviridae (e.g., vesiculovirus, lyssavirus, ephemerovirus, cytorhabdovirus, and necleorhabdovirus), arenaviridae (e.g., arenavirus, lymphocytic choriomeningitis virus, Ippy virus, and lassa virus), and coronaviridae (e.g., coronavirus and torovirus).

Specific examples of antibodies available useful for the treatment of a viral infectious disease include, but are not limited to, PRO542 (Progenics) which is a CD4 fusion antibody useful for the treatment of HIV infection; Ostavir (Protein Design Labs, Inc., CA) which is a human antibody useful for the treatment of hepatitis B virus; and Protovir (Protein Design Labs, Inc., CA) which is a humanized IgG1 antibody useful for the treatment of cytomegalovirus (CMV); and palivizumab (SYNAGIS®; MedImmune, Inc.; International Publication No. WO 02/43660) which is a humanized antibody useful for treatment of RSV.

In a specific embodiment, the anti-viral agents used in the compositions and methods of the invention inhibit or reduce a pulmonary or respiratory virus infection, inhibit or reduce the replication of a virus that causes a pulmonary or respiratory infection, or inhibit or reduce the spread of a virus that causes a pulmonary or respiratory infection to other cells or subjects. In another preferred embodiment, the anti-viral agents used in the compositions and methods of the invention inhibit or reduce infection by RSV, hMPV, or PIV, inhibit or reduce the replication of RSV, hMPV, or PIV, or inhibit or reduce the spread of RSV, HMPV, or PIV to other cells or subjects. Examples of such agents and methods of treatment of RSV, hMPV, and/or PIV infections include, but are not limited to, nucleoside analogs, such as zidovudine, acyclovir, gangcyclovir, vidarabine, idoxuridine, trifluridine, and ribavirin, as well as foscarnet, amantadine, rimantadine, saquinavir, indinavir, ritonavir, and the alpha-interferons as well as the nucleotide analog compounds 414B and 363B disclosed by Bond et al in International Patent Application WO 2005/061513.

In certain embodiments, methods and compositions of the invention are used to treat and/or prevent an infection with mammalian metapneumovirus and RSV. In an illustrative embodiment, an antibody that immunospecifically binds an RSV antigen and an antibody of the invention are co-administered to treat the infection with mammalian metapneumovirus and RSV. In certain embodiments, the anti-RSV-antigen antibody binds immunospecifically to an RSV antigen of the Group A of RSV. In other embodiments, the anti-RSV-antigen antibody binds immunospecifically to an RSV antigen of the Group B of RSV. In other embodiments, an antibody binds to an antigen of RSV of one Group and cross reacts with the analogous antigen of the other Group. In particular embodiments, the anti-RSV-antigen antibody binds immunospecifically to a RSV nucleoprotein, RSV phosphoprotein, RSV matrix protein, RSV small hydrophobic protein, RSV RNA-dependent RNA polymerase, RSV F protein, and/or RSV G protein. In additional specific embodiments, the anti-RSV-antigen antibody binds to allelic variants of a RSV nucleoprotein, a RSV nucleocapsid protein, a RSV phosphoprotein, a RSV matrix protein, a RSV attachment glycoprotein, a RSV fusion glycoprotein, a RSV nucleocapsid protein, a RSV matrix protein, a RSV small hydrophobic protein, a RSV RNA-dependent RNA polymerase, a RSV F protein, a RSV L protein, a RSV P protein, and/or a RSV G protein.

It should be recognized that antibodies that immunospecifically bind to an RSV antigen are known in the art. For example, palivizumab (SYNAGIS®) is a humanized monoclonal antibody presently used for the prevention of RSV infection in pediatric patients. In a specific embodiment, an antibody to be used with the methods of the present invention is palivizumab or an antibody-binding fragment thereof (e.g., a fragment containing one or more complementarity determining regions (CDRs) and preferably, the variable domain of palivizumab). The amino acid sequence of palivizumab is disclosed, e.g., in Johnson et al., 1997, J. Infectious Disease 176:1215-1224, and U.S. Pat. No. 5,824,307 and International Application Publication No.: WO 02/43660, entitled “Methods of Administering/Dosing Anti-RSV Antibodies for Prophylaxis and Treatment”, by Young et al., which are incorporated herein by reference in their entireties.

One or more antibodies or antigen-binding fragments thereof that bind immunospecifically to a RSV antigen comprise a Fc domain with a higher affinity for the FcRn receptor than the Fc domain of palivizumab can also be used in accordance with the invention. Such antibodies are described in U.S. patent application Ser. No. 10/020,354, filed Dec. 12, 2001, which is incorporated herein by reference in its entireties. Further, one or more of the anti-RSV-antigen antibodies A4B4; P12f2 P12f4; P11d4; Ale9; A12a6; A13c4; A17d4; A4B4; 1X-493L1; FR H3-3F4; M3H9; Y10H6; DG; AFFF; AFFF(1); 6H8; L1-7E5; L2-15B10; A13a11; A1h5; A4B4(1);A4B4-F52S; or A4B4L1FR-S28R can be used in accordance with the invention. These antibodies are disclosed in International Application Publication No.: WO 02/43660, entitled “Methods of Administering/Dosing Anti-RSV Antibodies for Prophylaxis and Treatment”, by Young et al., and US Provisional Patent Application 60/398,475 filed Jul. 25, 2002, entitled “Methods of Treating and Preventing RSV, HMPV, and PIV Using Anti-RSV, Anti-HMPV, and Anti-PIV Antibodies” which are incorporated herein by reference in their entireties.

In certain embodiments, the anti-RSV-antigen antibodies are the anti-RSV-antigen antibodies of or are prepared by the methods of U.S. application Ser. No: 09/724,531, filed Nov. 28, 2000; U.S. Ser. No. 09/996,288, filed Nov. 28, 2001; and U.S. Pat. Publication No. US2003/0091584 A1, published May 15, 2003, all entitled “Methods of Administering/Dosing Anti-RSV Antibodies for Prophylaxis and Treatment”, by Young et al., which are incorporated by reference herein in their entireties. Methods and composition for stabilized antibody formulations that can be used in the methods of the present invention are disclosed in U.S. Provisional Application Nos. 60/388,921, filed Jun. 14, 2002, and 60/388,920, filed Jun. 14, 2002, which are incorporated by reference herein in their entireties.

Anti-viral therapies and their dosages, routes of administration and recommended usage are known in the art and have been described in such literature as the Physician's Desk Reference (56th ed., 2002). Additional information on respiratory viral infections is available in Cecil Textbook of Medicine (18th ed., 1988).

5.2.2 Anti-Bacterial Agents

In certain embodiments, the antibodies of the invention, composition of the invention and methods of the invention can be used in combination with compositions and methods for the treatment and prevention of bacterial infections, such as bacterial infections of the pulmonary system in a mammal.

Anti-bacterial agents and therapies well known to one of skill in the art for the prevention, treatment, management, or amelioration of bacterial infections can be used in the compositions and methods of the invention. Non-limiting examples of anti-bacterial agents include proteins, polypeptides, peptides, fusion proteins, antibodies, nucleic acid molecules, organic molecules, inorganic molecules, and small molecules that inhibit or reduce a bacterial infection, inhibit or reduce the replication of bacteria, or inhibit or reduce the spread of bacteria to other subjects. In particular, examples of anti-bacterial agents include, but are not limited to, penicillin, cephalosporin, imipenem, axtreonam, vancomycin, cycloserine, bacitracin, chloramphenicol, erythromycin, clindamycin, tetracycline, streptomycin, tobramycin, gentamicin, amikacin, kanamycin, neomycin, spectinomycin, trimethoprim, norfloxacin, rifampin, polymyxin, amphotericin B, nystatin, ketocanazole, isoniazid, metronidazole, and pentamidine.

In a preferred embodiment, the anti-bacterial agent is an agent that inhibits or reduces a pulmonary or respiratory bacterial infection, inhibits or reduces the replication of a bacteria that causes a pulmonary or respiratory infection, or inhibits or reduces the spread of a bacteria that causes a pulmonary or respiratory infection to other subjects. In cases in which the pulmonary or respiratory bacterial infection is a mycoplasma infection (e.g., pharyngitis, tracheobronchitis, and pneumonia), the anti-bacterial agent is preferably a tetracycline, erythromycin, or spectinomycin. In cases in which the pulmonary or respiratory bacterial infection is pneumonia caused by aerobic gram negative bacilli (GNB), the anti-bacterial agent is preferably penicillin, first second, or third generation cephalosporin (e.g., cefaclor, cefadroxil, cephalexin, or cephazolin), erythomycin, clindamycin, an aminoglycoside (e.g., gentamicin, tobramycin, or amikacine), or a monolactam (e.g., aztreonam). In cases in which the pulmonary or respiratory bacterial infection is tuberculosis, the anti-bacterial agent is preferably, rifampcin, isonaizid, pyranzinamide, ethambutol, and streptomycin. In cases in which the respiratory infection is recurrent aspiration pneumonia, the anti-bacterial agent is preferably penicillin, an aminoglycoside, or a second or third generation cephalosporin.

Anti-bacterial therapies and their dosages, routes of administration and recommended usage are known in the art and have been described in such literature as the Physician's Desk Reference (56th ed., 2002). Additional information on respiratory infections and anti-bacterial therapies is available in Cecil Textbook of Medicine (18th ed., 1988).

5.2.8 Anti-Fungal Agents

In certain embodiments, the antibodies of the invention, composition of the invention and methods of the invention can be used in combination with compositions and methods for the treatment and prevention of fungal infections, such as fungal infections of the pulmonary system in a mammal.

Anti-fungal agents and therapies well known to one of skill in the art for prevention, management, treatment, and/or amelioration of a fungal infection or one or more symptoms thereof (e.g., a fungal respiratory infection) can be used in the compositions and methods of the invention. Non-limiting examples of anti-fungal agents include proteins, polypeptides, peptides, fusion proteins, antibodies, nucleic acid molecules, organic molecules, inorganic molecules, and small molecules that inhibit and/or reduce fungal infection, inhibit and/or reduce the replication of fungi, or inhibit and/or reduce the spread of fungi to other subjects. Specific examples of anti-fungal agents include, but are not limited to, azole drugs (e.g., miconazole, ketoconazole (NIZORAL®), caspofungin acetate (CANCIDAS®), imidazole, triazoles (e.g., fluconazole (DIFLUCAN®)), and itraconazole (SPORANOX®)), polyene (e.g., nystatin, amphotericin B (FUNGIZONE®), amphotericin B lipid complex (“ABLC”)(ABELCET®), amphotericin B colloidal dispersion (“ABCD”)(AMPHOTEC®), liposomal amphotericin B (AMBISONE®)), potassium iodide (KI), pyrimidine (e.g., flucytosine (ANCOBON®)), and voriconazole (VFEND®).

In certain embodiments, the anti-fungal agent is an agent that inhibits or reduces a respiratory fungal infection, inhibits or reduces the replication of a fungus that causes a pulmonary or respiratory infection, or inhibits or reduces the spread of a fungus that causes a pulmonary or respiratory infection to other subjects. In cases in which the pulmonary or respiratory fungal infection is Blastomyces dermatitidis, the anti-fungal agent is preferably itraconazole, amphotericin B, fluconazole, or ketoconazole. In cases in which the pulmonary or respiratory fungal infection is pulmonary aspergilloma, the anti-fungal agent is preferably amphotericin B, liposomal amphotericin B, itraconazole, or fluconazole. In cases in which the pulmonary or respiratory fungal infection is histoplasmosis, the anti-fungal agent is preferably amphotericin B, itraconazole, fluconazole, or ketoconazole. In cases in which the pulmonary or respiratory fungal infection is coccidioidomycosis, the anti-fungal agent is preferably fluconazole or amphotericin B. In cases in which the pulmonary or respiratory fungal infection is cryptococcosis, the anti-fungal agent is preferably amphotericin B, fluconazole, or combination of the two agents. In cases in which the pulmonary or respiratory fungal infection is chromomycosis, the anti-fungal agent is preferably itraconazole, fluconazole, or flucytosine. In cases in which the pulmonary or respiratory fungal infection is mucormycosis, the anti-fungal agent is preferably amphotericin B or liposomal amphotericin B. In cases in which the pulmonary or respiratory fungal infection is pseudoallescheriasis, the anti-fungal agent is preferably itraconazole ore miconazole.

Anti-fungal therapies and their dosages, routes of administration, and recommended usage are known in the art and have been described in such literature as Dodds et al., 2000 Pharmacotherapy 20(11) 1335-1355, the Physician's Desk Reference (57th ed., 2003) and the Merck Manual of Diagnosis and Therapy (17th ed., 1999).

5.3 Prophylactic & Therapeutic Uses of Antibodies

The present invention is directed to therapies which involve administering one of more antibodies of the invention and compositions comprising said antibodies to a subject, preferably a human subject, for preventing, treating, managing, and/or ameliorating disease or disorder or one or more symptoms thereof. In one embodiment, the invention provides a method of preventing, treating, managing, and/or ameliorating a disease or disorder or one or more symptoms thereof, said method comprising administering to a subject in need thereof an effective amount of one or more antibodies of the invention. In certain embodiments, an effective amount of one or more polypeptides, peptides, and proteins comprising one or more antibodies or antibody fragments of the invention is administered to a subject in need thereof to prevent, treat, manage, and/or ameliorate an infection with mammalian metapneumovirus or one or more symptoms thereof.

The invention also provides methods of preventing, treating, managing, and/or ameliorating a disease or disorder or one or more symptoms thereof, said methods comprising administering to a subject in need thereof one or more of the antibodies of the invention and one or more therapies (e.g., one or more prophylactic or therapeutic agents) other than antibodies of the invention that are currently being used, have been used, or are known to be useful in the prevention, treatment, management, and/or amelioration of an infection with mammalian metapneumovirus or an infection with mammalian metapneumovirus and one or more other infectious agents or one or more symptoms of such an infection.

The prophylactic or therapeutic agents of the combination therapies of the invention can be administered sequentially or concurrently. In a specific embodiment, the combination therapies of the invention comprise an effective amount of one or more antibodies of the invention and an effective amount of at least one other therapy that also targets mammalian metapneumovirus. In another specific embodiment, the combination therapies of the invention comprise an effective amount of one or more antibodies of the invention and an effective amount of at least one other therapy that targets an infectious agent other than mammalian metapneumovirus. In certain embodiments, the combination therapies of the present invention improve the prophylactic or therapeutic effect of one or more antibodies of the invention by functioning together with the antibodies to have an additive or synergistic effect. In certain embodiments, the combination therapies of the present invention reduce the side effects associated with the prophylactic or therapeutic agents.

The prophylactic or therapeutic agents of the combination therapies can be administered to a subject, preferably a human subject, in the same pharmaceutical composition. Alternatively, the prophylactic or therapeutic agents of the combination therapies can be administered concurrently to a subject in separate pharmaceutical compositions. The prophylactic or therapeutic agents may be administered to a subject by the same or different routes of administration.

The following sections describe how the antibodies of the invention, the compositions of the invention, and the methods of the invention can be used with other treatments to provide a broad spectrum antibiotic treatment. Without being bound by theory, certain viral infections, such as RSV infections, may cause symptoms similar to the symptoms caused by an infection with mammalian metapneumovirus. Thus, in situations where an infection with mammalian metapneumovirus and one or more other infectious agents has been diagnosed or in situations where an infection with mammalian metapneumovirus and one or more other infectious agents is a possibility, an antibody of the invention can be administered together with an additional therapy to treat the infection with the other infectious agent.

5.3.1 Viral Infections in Addition to Infections with Mammalian Metapneumovirus

One or more antibodies of the invention and compositions comprising said antibodies can be administered to a subject to prevent, treat, manage, and/or ameliorate a viral infection with a mammalian metapneumovirus or one or more symptoms thereof. Further, one or more antibodies of the invention and compositions comprising said antibodies may be administered in combination with one or more other therapies (e.g., one or more prophylactic or therapeutic agents) for the prevention, treatment, management, or amelioration of a viral infection with a mammalian metapneumovirus and one or more other viruses.

In certain embodiments, an effective amount of one or more antibodies of the invention is administered in combination with an effective amount of one or more therapies (e.g., one or more prophylactic or therapeutic agents) currently being used, have been used, or are known to be useful in the prevention, management, treatment, and/or amelioration of a viral infection, preferably a viral respiratory infection, or one or more symptoms thereof to a subject in need thereof. Therapies for a viral infection, preferably a viral respiratory infection include, but are not limited to, anti-viral agents such as amantadine, oseltamivir, ribaviran, palivizumab, and anamivir. In certain embodiments, an effective amount of one or more antibodies of the invention is administered in combination with one or more supportive measures to a subject in need thereof to prevent, manage, treat, and/or ameliorate a viral infection or one or more symptoms thereof. Non-limiting examples of supportive measures include humidification of the air by an ultrasonic nebulizer, aerolized racemic epinephrine, oral dexamethasone, intravenous fluids, intubation, fever reducers (e.g., ibuprofen, acetometaphin), and antibiotic and/or anti-fungal therapy (i.e., to prevent or treat secondary bacterial infections).

Any type of viral infection or condition resulting from or associated with a viral infection (e.g., a respiratory condition) can be prevented, treated, managed, and/or ameliorated in accordance with the methods of the invention, said methods comprising administering an effective amount of one or more antibodies of the invention alone or in combination with an effective amount of another therapy (e.g., a prophylactic or therapeutic agent other than antibodies of the invention). Examples of viruses which cause viral infections include, but are not limited to, retroviruses (e.g., human T-cell lymphotrophic virus (HTLV) types I and II and human immunodeficiency virus (HIV)), herpes viruses (e.g., herpes simplex virus (HSV) types I and II, Epstein-Barr virus, HHV6-HHV8, and cytomegalovirus), arenavirues (e.g., lassa fever virus), paramyxoviruses (e.g., morbillivirus virus, human respiratory syncytial virus, mumps, hMPV, and pneumovirus), adenoviruses, bunyaviruses (e.g., hantavirus), cornaviruses, filoviruses (e.g., Ebola virus), flaviviruses (e.g., hepatitis C virus (HCV), yellow fever virus, and Japanese encephalitis virus), hepadnaviruses (e.g., hepatitis B viruses (HBV)), orthomyoviruses (e.g., influenza viruses A, B and C and PIV), papovaviruses (e.g., papillomavirues), picomaviruses (e.g., rhinoviruses, enteroviruses and hepatitis A viruses), poxviruses, reoviruses (e.g., rotavirues), togaviruses (e.g., rubella virus), and rhabdoviruses (e.g., rabies virus). Biological responses to a viral infection include, but not limited to, elevated levels of IgE antibodies, increased proliferation and/or infiltration of T cells, increased proliferation and/or infiltration of B cells, epithelial hyperplasia, and mucin production. In a specific embodiment, the invention also provides methods of preventing, treating, managing, and/or ameliorating viral respiratory infections that are associated with or cause the common cold, viral pharyngitis, viral laryngitis, viral croup, viral bronchitis, influenza, parainfluenza viral diseases (“PIV”) diseases (e.g., croup, bronchiolitis, bronchitis, pneumonia), respiratory syncytial virus (“RSV”) diseases, metapneumavirus diseases, and adenovirus diseases (e.g., febrile respiratory disease, croup, bronchitis, pneumonia), said method comprising administering an effective amount of one or more antibodies of the invention alone or in combination with an effective amount of another therapy.

In a specific embodiment, a mammalian metapneumovirus infection together with influenza virus infections, PIV infections, adenovirus infections, and/or RSV infections, or one or more of symptoms thereof are prevented, treated, managed, and/and/or ameliorated in accordance with the methods of the invention. In a specific embodiment, the invention provides methods for preventing, treating, managing, and/or ameliorating a RSV respiratory infection or one or more symptoms thereof, said methods comprising administering to a subject in need thereof an effective amount of one or more antibodies of the invention alone or in combination with one or more anti-viral agents such as, but not limited to, amantadine, rimantadine, oseltamivir, znamivir, ribaviran, RSV-IVIG (i.e., intravenous immune globulin infusion) (RESPIGAM™), the nucleotide analog compounds 414B and 363B disclosed by Bond et al in International Patent Application WO 2005/061513, and palivizumab. In a specific embodiment, the invention provides methods for preventing, treating, managing, and/or ameliorating a PIV infection or one or more symptoms thereof, said methods comprising administering to a subject in need thereof an effective amount of one or more antibodies of the invention alone or in combination with an effective amount of one or more anti-viral agents such as, but not limited to, amantadine, rimantadine, oseltamivir, znamivir, ribaviran, and palivizumab. In another specific embodiment, the invention provides methods for preventing, treating, managing, and/or ameliorating a hMPV infection or one or more symptoms thereof, said methods comprising of administering an effective amount of one or more antibodies of the invention alone or in combination with an effective amount of one or more anti-viral agents, such as, but not limited to, amantadine, rimantadine, oseltamivir, znamivir, ribaviran, and palivizumab to a subject in need thereof. In a specific embodiment, the invention provides methods for preventing, treating,.managing, and/or ameliorating influenza, said methods comprising administering an effective amount of one or more antibodies of the invention alone or in combination with an effective amount of an anti-viral agent such as, but not limited to zanamivir (RELENZA®), oseltamivir (TAMIFLU®), rimantadine, and amantadine (SYMADINE®; SYMMETREL®) to a subject in need thereof.

Viral infection therapies and their dosages, routes of administration and recommended usage are known in the art and have been described in such literature as the Physician's Desk Reference (57th ed., 2003).

5.3.2 Bacterial Infections

The invention provides a method of preventing, treating, managing, and/or ameliorating an infection with mammalian metapneumovirus together with an infection with a bacterial infection or one or more symptoms of such an infection, said method comprising administering to a subject in need thereof an effective amount of one or more antibodies of the invention in combination with an anti-bacterial agent.

Any type of bacterial infection or condition resulting from or associated with a bacterial infection (e.g., a respiratory infection) can be prevented, treated, managed, and/or ameliorated in accordance with the methods of invention. Examples of bacteria which cause bacterial infections include, but not limited to, the Aquaspirillum family, Azospirillum family, Azotobacteraceae family, Bacteroidaceae family, Bartonella species, Bdellovibrio family, Campylobacter species, Chlamydia species (e.g., Chlamydia pneumoniae), clostridium, Enterobacteriaceae family (e.g., Citrobacter species, Edwardsiella, Enterobacter aerogenes, Erwinia species, Escherichia coli, Hafnia species, Klebsiella species, Morganella species, Proteus vulgaris, Providencia, Salmonella species, Serratia marcescens, and Shigella flexneri), Gardinella family, Haemophilus influenzae, Halobacteriaceae family, Helicobacter family, Legionallaceae family, Listeria species, Methylococcaceae family, mycobacteria (e.g., Mycobacterium tuberculosis), Neisseriaceae family, Oceanospirillum family, Pasteurellaceae family, Pneumococcus species, Pseudomonas species, Rhizobiaceae family, Spirillum family, Spirosomaceae family, Staphylococcus (e.g., methicillin resistant Staphylococcus aureus and Staphylococcus pyrogenes), Streptococcus (e.g., Streptococcus enteritidis, Streptococcus fasciae, and Streptococcus pneumoniae), Vampirovibr Helicobacter family, and Vampirovibrio family.

In certain embodiments, the invention provides methods to prevent, treat, manage, and/or ameliorate an infection with a mammalian metapneumovirus together with a bacterial infection, preferably a bacterial respiratory infection, or one or more of the symptoms thereof, said methods comprising administering to a subject in need thereof one or more antibodies of the invention in combination with and effective amount of one or more therapies (e.g., one or more prophylactic or therapeutic agents), other than antibodies of the invention, used to prevent, treat, manage, and/or ameliorate bacterial infections. Therapies for bacterial infections, particularly, bacterial respiratory infections include, but are not limited to, anti-bacterial agents (e.g., aminoglycosides (e.g., gentamicin, tobramycin, amikacin, netilimicin) aztreonam, celphalosporins (e.g., cefaclor, cefadroxil, cephalexin, cephazolin), clindamycin, erythromycin, penicillin (e.g., penicillin V, crystalline penicillin G, procaine penicillin G), spectinomycin, and tetracycline (e.g., chlortetracycline, doxycycline, oxytetracycine)) and supportive respiratory therapy, such as supplemental and mechanical ventilation. In certain embodiments, one or more antibodies of the invention are administered in combination with one or more supportive measures to a subject in need thereof to prevent, manage, treat, and/or ameliorate a bacterial infection or one or more symptoms thereof. Non-limiting examples of supportive measures include humidification of air by ultrasonic nebulizer, aerolized racemic epinephrine, oral dexamethasone, intravenous fluids, intubation, fever reducers (e.g., ibuprofen, acetometaphin), and more preferably, antibiotic or anti-viral therapy (i.e., to prevent or treat secondary infections).

In a specific embodiment, the methods of the invention are utilized to prevent, treat, manage, and/or ameliorate an infection with mammalian metapneumovirus together with an infection with a bacterial respiratory infection caused by Pneumonococcus, Mycobacteria, aerobic gram-negative bacilli, Streptococcus, or Hemophilus or one or more symptoms thereof, said method comprising administering to a subject in need thereof of an effective amount of one or more antibodies of the invention in combination with an effective amount of one or more other therapies (e.g., one or more prophylactic or therapeutic agents) other than antibodies of the invention.

Bacterial infection therapies and their dosages, routes of administration and recommended usage are known in the art and have been described in such literature as the Physician's Desk Reference (57th ed., 2003).

5.3.3 Fungal Infections

The invention provides a method of preventing, treating, managing, and/or ameliorating an infection with mammalian metapneumovirus together with an infection with a fungus or one or more symptoms of such an infection, said method comprising administering to a subject in need thereof an effective amount of one or more antibodies of the invention in combination with an anti-fungal agent. One or more antibodies of the invention together with an anti-fungal agent can be administered to a subject to prevent, treat, manage, and/or ameliorate an infection with a mammalian metapneumovirus and a fungus or one or more symptoms of such an infection.

Any type of fungal infection or condition resulting from or associated with a fungal infection (e.g., a respiratory infection) can be prevented, treated, managed, and/or ameliorated in combination with the prevention, treatment, management, and/or amelioration of an infection with mammalian metapneumovirus. Examples of fungus which cause fungal infections include, but not limited to, Absidia species (e.g., Absidia corymbifera and Absidia ramosa), Aspergillus species, (e.g., Aspergillus flavus, Aspergillus fumigatus, Aspergillus nidulans, Aspergillus niger, and Aspergillus terreus), Basidiobolus ranarum, Blastomyces dermatitidis,Candida species (e.g., Candida albicans, Candida glabrata, Candida kerr, Candida krusei, Candida parapsilosis, Candida pseudotropicalis, Candida quillermondii, Candida rugosa, Candida stellatoidea, and Candida tropicalis), Coccidioides immitis, Conidiobolus species, Cryptococcus neoforms, Cunninghamella species, dermatophytes, Histoplasma capsulatum, Microsporum gypseum, Mucor pusillus, Paracoccidioides brasiliensis, Pseudallescheria boydii, Rhinosporidium seeberi, Pneumocystis carinii, Rhizopus species (e.g., Rhizopus arrhizus, Rhizopus oryzae, and Rhizopus microsporus), Saccharomyces species, Sporothrix schenckii, zygomycetes, and classes such as Zygomycetes, Ascomycetes, the Basidiomycetes, Deuteromycetes, and Oomycetes.

In certain embodiments, an effective amount of one or more antibodies is administered in combination with an effective amount of one or more therapies (e.g., one or more prophylactic or therapeutic agents), other than antibodies of the invention, which are currently being used, have been used, or are known to be useful in the prevention, management, treatment, or amelioration of a fungal infection, preferably a fungal respiratory infection, to a subject in need thereof. Therapies for fungal infections include, but are not limited to, anti-fungal agents such as azole drugs e.g., miconazole, ketoconazole (NIZORAL®), caspofungin acetate (CANCIDAS®), imidazole, triazoles (e.g., fluconazole (DIFLUCAN®)), and itraconazole (SPORANOX®)), polyene (e.g., nystatin, amphotericin B colloidal dispersion (“ABCD”)(AMPHOTEC®), liposomal amphotericin B (AMBISONE®)), postassium iodide (KI), pyrimidine (e.g., flucytosine (ANCOBON®)), and voriconazole (VFEND®). In certain embodiments, an effective amount of one or more antibodies of the invention are administered in combination with one or more supportive measures to a subject in need thereof to prevent, manage, treat, and/or ameliorate a fungal infection or one or more symptoms thereof. Non-limiting examples of supportive measures include humidification of the air by an ultrasonic nebulizer, aerolized racemic epinephrine, oral desamethasone, intravenous fluids, intubation, fever reducers (e.g., ibuprofen and acetometaphin), and anti-viral or anti-bacterial therapy (i.e., to prevent or treat secondary viral or bacterial infections).

Fungal infection therapies and their dosages, routes of administration and recommended usage are known in the art and have been described in such literature as the Physician's Desk Reference (57th ed., 2003).

5.4 Compositions & Methods of Administering Antibodies

In certain embodiments, a composition of the invention comprises one or more antibodies of the invention or a fragment thereof, wherein the antibody and the fragment each immunospecifically bind to an F protein of a mammalian metapneumovirus. In another embodiment, a composition comprises one or more antibodies of the invention and one or more prophylactic or therapeutic agents, other than the antibodies of the invention, said agents known to be useful for or having been or currently used for the prevention, treatment, management, and/or amelioration of infectious diseases, such as viral infections, bacterial infections, and fungal infections.

In one embodiment, a composition comprises one or more peptides, polypeptides, or proteins comprising a fragment of an antibody of the invention that immunospecifically binds to an F protein of a mammalian metapneumovirus. In another embodiment, a compositions comprises one or more peptides, polypeptides, or proteins comprising a fragment of an antibody of the invention that immunospecifically binds to an F protein of a mammalian metapneumovirus in combination with one or more other therapies (e.g., one or more prophylactic or therapeutic agents), other than a peptide, polypeptide, or protein comprising a fragment of an antibody of the invention.

In a specific embodiment, a composition of the invention further comprises one or more immunomodulatory agents, one or more anti-viral agents, one or more anti-bacterial agents, one or more anti-fungal agents, and/or an anti-inflammatory agent.

The compositions of the invention include bulk drug compositions useful in the manufacture of pharmaceutical compositions (e.g., compositions that are suitable for administration to a subject or patient) which can be used in the preparation of unit dosage forms. In a preferred embodiment, a composition of the invention is a pharmaceutical composition. Such compositions comprise a prophylactically or therapeutically effective amount of one or more prophylactic or therapeutic agents (e.g., an antibody of the invention; polypeptide, peptide, or protein comprising an antibody fragment of the invention, or other prophylactic or therapeutic agent), and a pharmaceutically acceptable carrier. Preferably, the pharmaceutical compositions are formulated to be suitable for the route of administration to a subject. In illustrative, non-limiting, embodiments, a phamaceutical composition of the invention is formulated in single dose vials as a sterile liquid that contains 10 mM histidine buffer at pH 6.0 and 150 mM sodium chloride. Each 1.0 mL of solution contains 100 mg of protein, 1.6 mg of histidine and 8.9 mg of sodium chloride in water for optimal stability and solubility.

In a specific embodiment, a “pharmaceutically acceptable” carrier is approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like.

Generally, the ingredients of compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Various delivery systems are known in the art and can be used to administer a prophylactic or therapeutic agent or composition of the invention to prevent, treat, manage, and/or ameliorate a disease or disorder associated with an infection with a mammalian metapneumovirus. Methods of administering a therapy (e.g., prophylactic or therapeutic agent) of the invention include, but are not limited to, parenteral administration (e.g., intradermal, intramuscular, intraperitoneal, intravenous and subcutaneous), epidurala administration, intratumoral administration, and mucosal adminsitration (e.g., intranasal and oral routes). In addition, pulmonary administration can also be employed, e.g., by use of an inhaler or nebulizer, and formulation with an aerosolizing agent. See, e.g., U.S. Pat. Nos. 6,019,968, 5,985, 320, 5,985,309, 5,934,272, 5,874,064, 5,855,913, 5,290,540, and 4,880,078; and PCT Publication Nos. WO 92/19244, WO 97/32572, WO 97/44013, WO 98/31346, and WO 99/66903, each of which is incorporated herein by reference their entirety. In one embodiment, an anitbody, combination therapy, or a composition of the invention is administered using Alkermes AIR™ pulmonary drug delivery technology (Alkermes, Inc., Cambridge, Mass.). In a specific embodiment, prophylactic or therapeutic agents of the invention are administered intramuscularly, intravenously, intratumorally, orally, intranasally, pulmonary, or subcutaneously. The prophylactic or therapeutic agents may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

In a specific embodiment, it may be desirable to administer the prophylactic or therapeutic agents of the invention locally to the area in need of treatment; this may be achieved by, for example, and not by way of limitation, local infusion, by injection, or by means of an implant, said implant being of a porous or non-porous material, including membranes and matrices, such as sialastic membranes, polymers, fibrous matrices (e.g., Tissuel®), or collagen matrices. In one embodiment, an effective amount of one or more antibodies of the invention is administered locally to the affected area to a subject at risk of or with a disease or disorder associated with an infection with mammalian metapneumovirus. In another embodiment, an effective amount of one or more antibodies of the invention is administered locally to the affected area in combination with an effective amount of one or more therapies (e.g., one or more prophylactic or therapeutic agents) other than an antibody of the invention to a subject at risk of or with a disease or disorder associated with or characterized by infection with mammalian metapneumovirus and another infectious agent.

In yet another embodiment, a therapy of the invention can be delivered in a controlled release or sustained release system. In one embodiment, a pump may be used to achieve controlled or sustained release (see Langer, supra; Sefton, 1987, CRC Crit. Ref. Biomed. Eng. 14:20; Buchwald et al., 1980, Surgery 88:507; Saudek et al., 1989, N. Engl. J. Med. 321:574). In another embodiment, polymeric materials can be used to achieve controlled or sustained release of the therapies of the invention (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J., Macromol. Sci. Rev. Macromol. Chem. 23:61; see also Levy et al., 1985, Science 228:190; During et al., 1989, Ann. Neurol. 25:351; Howard et al., 1989, J. Neurosurg. 7 1:105); U.S. Pat. No. 5,679,377; U.S. Pat. No. 5,916,597; U.S. Pat. No. 5,912,015; U.S. Pat. No. 5,989,463; U.S. Pat. No. 5,128,326; PCT Publication No. WO 99/15154; and PCT Publication No. WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In a preferred embodiment, the polymer used in a sustained release formulation is inert, free of leachable impurities, stable on storage, sterile, and biodegradable. In yet another embodiment, a controlled or sustained release system can be placed in proximity of the prophylactic or therapeutic target, thus requiring only a fraction of the systemic dose (see, e.g., Goodson, in Medical Applications of Controlled Release, supra, vol. 2, pp. 115-138 (1984)).

Controlled release systems are discussed in the review by Langer (1990, Science 249:1527-1533). Any technique known to one of skill in the art can be used to produce sustained release formulations comprising one or more therapeutic agents of the invention. See, e.g., U.S. Pat. No. 4,526,938, PCT publication WO 91/05548, PCT publication WO 96/20698,.Ning et al., 1996, “Intratumoral Radioimmunotheraphy of a Human Colon Cancer Xenograft Using a Sustained Release Gel,” Radiotherapy & Oncology 39:179 189, Song et al., 1995, “Antibody Mediated Lung Targeting of Long Circulating Emulsions,” PDA Journal of Pharmaceutical Science & Technology 50:372 397, Cleek et al., 1997, “Biodegradable Polymeric Carriers for a bFGF Antibody for Cardiovascular Application,” Pro. Int'l. Symp. Control. Rel. Bioact. Mater. 24:853 854, and Lam et al., 1997, “Microencapsulation of Recombinant Humanized Monoclonal Antibody for Local Delivery,” Proc. Int'l. Syrup. Control Rel. Bioact. Mater. 24:759 760, each of which is incorporated herein by reference in their entirety.

In certain embodiments, a nucleic acid encoding an antibody of the invention or a frament thereof can be administered to a subject to treat and/or prevent an infection with mammalian metapneumovirus. Further, a cell transfected with a nucleic acid encoding an antibody of the invention or a frament thereof can be administered to a subject to treat and/or prevent an infection with mammalian metapneumovirus. Thus, the invention also provides compositions comprising a nucleic acid encoding an antibody of the invention or a fragment thereof. In a specific embodiment, where the composition of the invention is a nucleic acid, the nucleic acid can be administered to promote expression of its encoded antibody or fragment thereof, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see, e.g., Joliot et al., 1991, Proc. Natl. Acad. Sci. USA 88:1864-1868). In certain embodiments, a nucleic acid can be introduced intracellularly and incorporated within host cell DNA for expression by homologous recombination. Further, the nucleic acid can be introduced into a host cell ex vivo and the host cell with the nucleic acid encoding an antibody of the invention or a fragment thereof is administered to the subject in need of treatment and/or prevention of an infection with a mammalian metapneumovirus.

A pharmaceutical composition of the invention is formulated to be compatible with its intended route of administration. Examples of routes of administration include, but are not limited to, parenteral, e.g., intravenous, intradermal, subcutaneous, oral, intranasal (e.g., inhalation), transdermal (e.g., topical), transmucosal, and rectal administration. In a specific embodiment, the composition is formulated in accordance with routine procedures as a pharmaceutical composition adapted for intravenous, subcutaneous, intramuscular, oral, intranasal, or topical administration to human beings. Typically, compositions for intravenous administration are solutions in sterile isotonic aqueous buffer. Where necessary, the composition may also include a solubilizing agent and a local anesthetic such as lignocamne to ease pain at the site of the injection.

If the compositions of the invention are to be administered topically, the compositions can be formulated in the form of an ointment, cream, transdermal patch, lotion, gel, shampoo, spray, aerosol, solution, emulsion, or other form well-known to one of skill in the art. See, e.g., Remington's Pharmaceutical Sciences and Introduction to Pharmaceutical Dosage Forms, 19th ed., Mack Pub. Co., Easton, Pa. (1995). For non-sprayable topical dosage forms, viscous to semi-solid or solid forms comprising a carrier or one or more excipients compatible with topical application and having a dynamic viscosity preferably greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, preferably in combination with a solid or liquid inert carrier, is packaged in a mixture with a pressurized volatile (e.g., a gaseous propellant, such as freon) or in a squeeze bottle. Moisturizers or humectants can also be added to pharmaceutical compositions and dosage forms if desired. Examples of such additional ingredients are well-known in the art.

If the method of the invention comprises intranasal administration of a composition, the composition can be formulated in an aerosol form, spray, mist or in the form of drops. In particular, prophylactic or therapeutic agents for use according to the present invention can be conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). In the case of a pressurized aerosol the dosage unit may be determined by providing a valve to deliver a metered amount. Capsules and cartridges (composed of, e.g., gelatin) for use in an inhaler or insufflator may be formulated containing a powder mix of the compound and a suitable powder base such as lactose or starch.

If the method of the invention comprises oral administration, compositions can be formulated orally in the form of tablets, capsules, cachets, gelcaps, solutions, suspensions, and the like. Tablets or capsules can be prepared by conventional means with pharmaceutically acceptable excipients such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulphate). The tablets may be coated by methods well-known in the art. Liquid preparations for oral administration may take the form of, but not limited to, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated for slow release, controlled release, or sustained release of a prophylactic or therapeutic agent(s).

The method of the invention may comprise pulmonary administration, e.g., by use of an inhaler or nebulizer, of a composition formulated with an aerosolizing agent. See, e.g., U.S. Pat. Nos. 6,019,968, 5,985, 320, 5,985,309, 5,934,272, 5,874,064, 5,855,913, 5,290,540, and 4,880,078; and PCT Publication Nos. WO 92/19244, WO 97/32572, WO 97/44013, WO 98/31346, and WO 99/66903, each of which is incorporated herein by reference their entirety. In a specific embodiment, an antibody of the invention, combination therapy, and/or composition of the invention is administered using Alkermes AIR™ pulmonary drug delivery technology (Alkermes, Inc., Cambridge, Mass.).

The method of the invention may comprise administration of a composition formulated for parenteral administration by injection (e.g., by bolus injection or continuous infusion). Formulations for injection may be presented in unit dosage form (e.g., in ampoules or in multi-dose containers) with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle (e.g., sterile pyrogen-free water) before use.

The methods of the invention may additionally comprise of administration of compositions formulated as depot preparations. Such long acting formulations may be administered by implantation (e.g., subcutaneously or intramuscularly) or by intramuscular injection. Thus, for example, the compositions may be formulated with suitable polymeric or hydrophobic materials (e.g., as an emulsion in an acceptable oil) or ion exchange resins, or as sparingly soluble derivatives (e.g., as a sparingly soluble salt).

In particular, the invention also provides that one or more of the prophylactic or therapeutic agents, or pharmaceutical compositions of the invention is packaged in a hermetically sealed container such as an ampoule or sachette indicating the quantity of the agent. In one embodiment, one or more of the prophylactic or therapeutic agents, or pharmaceutical compositions of the invention is supplied as a dry sterilized lyophilized powder or water free concentrate in a hermetically sealed container and can be reconstituted (e.g., with water or saline) to the appropriate concentration for administration to a subject. Preferably, one or more of the prophylactic or therapeutic agents or pharmaceutical compositions of the invention is supplied as a dry sterile lyophilized powder in a hermetically sealed container at a unit dosage of at least 5 mg, more preferably at least 10 mg, at least 15 mg, at least 25 mg, at least 35 mg, at least 45 mg, at least 50 mg, at least 75 mg, or at least 100 mg. The lyophilized prophylactic or therapeutic agents or pharmaceutical compositions of the invention should be stored at between 2° C. and 8° C. in its original container and the prophylactic or therapeutic agents, or pharmaceutical compositions of the invention should be administered within 1 week, preferably within 5 days, within 72 hours, within 48 hours, within 24 hours, within 12 hours, within 6 hours, within 5 hours, within 3 hours, or within 1 hour after being reconstituted. In an alternative embodiment, one or more of the prophylactic or therapeutic agents or pharmaceutical compositions of the invention is supplied in liquid form in a hermetically sealed container indicating the quantity and concentration of the agent. Preferably, the liquid form of the administered composition is supplied in a hermetically sealed container at least 0.25 mg/ml, more preferably at least 0.5 mg/ml, at least 1 mg/ml, at least 2.5 mg/ml, at least 5 mg/ml, at least 8 mg/ml, at least 10 mg/ml, at least 15 mg/kg, at least 25 mg/ml, at least 50 mg/ml, at least 75 mg/ml or at least 100 mg/ml. The liquid form should be stored at between 2° C. and 8° C. in its original container.

Generally, an antibody of the invention that is to be administered to a subject in need of treatment or prevention of an infection with mammalian metapneumovirus is compatible with the species of the subject. Thus, in a preferred embodiment, human or humanized antibodies are administered to a human patient for therapy or prophylaxis. In a specific embodiment, the constant region of the antibody to be administered to a subject are identical to the amino acid sequence of the constant regions of the autologous antibodies in the subject.

5.4.1 Gene Therapy

In a specific embodiment, nucleotide sequences comprising nucleic acids encoding an antibody of the invention or another prophylactic or therapeutic agent are administered to treat, prevent, manage, and/or ameliorate infection with mammalian metapneumovirus or one or more symptoms thereof by way of gene therapy. Gene therapy refers to therapy performed by the administration to a subject of an expressed or expressible nucleic acid. In this embodiment of the invention, the nucleic acids produce their encoded antibody of the invention.

Any of the methods for gene therapy available in the art can be used according to the present invention. For general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, Science 260:926-932 (1993); and Morgan and Anderson, 1993, Ann. Rev. Biochem. 62:191-217; May, 1993, TIBTECH 11(5): 155-215. Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

In one embodiment, the method of the invention comprises administration of a composition comprising nucleic acids encoding an antibody of the invention or a fragment thereof, said nucleic acids being part of an expression vector. In particular, such nucleic acids have promoters, preferably heterologous promoters, operably linked to the antibody coding region, said promoter being inducible or constitutive, and, optionally, tissue-specific. In another embodiment, nucleic acid molecules are used in which the coding sequences of an antibody of the invention or another prophylactic or therapeutic agent and any other desired sequences are flanked by regions that promote homologous recombination at a desired site in the genome, thus providing for intrachromosomal expression of the antibody encoding nucleic acids (Koller and Smithies, 1989, Proc. Natl. Acad. Sci. USA 86:8932-8935; Zijlstra et al., 1989, Nature 342:435-438). In specific embodiments, the expressed antibody of the invention or other prophylactic or therapeutic agent is a single chain antibody; alternatively, the nucleic acid sequences include sequences encoding both the heavy and light chains, or fragments thereof, of the antibody of the invention or another prophylactic or therapeutic agent.

Delivery of the nucleic acids into a subject may be either direct, in which case the subject is directly exposed to the nucleic acid or nucleic acid-carrying vectors, or indirect, in which case, cells are first transformed with the nucleic acids in vitro, then transplanted into the subject. These two approaches are known, respectively, as in vivo or ex vivo gene therapy.

In a specific embodiment, the nucleic acid sequences are directly administered in vivo, where they are expressed to produce the encoded product. This can be accomplished by any of numerous methods known in the art, e.g., by constructing them as part of an appropriate nucleic acid expression vector and administering it so that they become intracellular, e.g., by infection using defective or attenuated retrovirals or other viral vectors (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, 1987, J. Biol. Chem. 262:4429-4432) (which can be used to target cell types specifically expressing the receptors). In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., International Publication Nos. WO 92/06180; WO 92/22635; W092/20316; W093/14188; and WO 93/20221). Alternatively, the nucleic acid can be introduced intracellularly and incorporated within host cell DNA for expression, by homologous recombination (Koller and Smithies, 1989, Proc. Natl. Acad. Sci. USA 86:8932-8935; and Zijlstra et al., 1989, Nature 342:435-438).

In a specific embodiment, viral vectors that contains nucleic acid sequences encoding an antibody of the invention or a fragment thereof are used as expression vectors for the antibody or fragment thereof. For example, a retroviral vector can be used (see Miller et al., 1993, Meth. Enzymol. 217:581-599). These retroviral vectors contain the components necessary for the correct packaging of the viral genome and integration into the host cell DNA. The nucleic acid sequences encoding an antibody of the invention or another prophylactic or therapeutic agent to be used in gene therapy are cloned into one or more vectors, which facilitates delivery of the gene into a subject. More detail about retroviral vectors can be found in Boesen et al., 1994, Biotherapy 6:291-302, which describes the use of a retroviral vector to deliver the mdr 1 gene to hematopoietic stem cells in order to make the stem cells more resistant to chemotherapy. Other references illustrating the use of retroviral vectors in gene therapy are: Clowes et al., 1994, J. Clin. Invest. 93:644-651; Klein et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, 1993, Current Opinion in Genetics and Development 3:499-503 present a review of adenovirus-based gene therapy. Bout et al., 1994, Human Gene Therapy 5:3-10 demonstrated the use of adenovirus vectors to transfer genes to the respiratory epithelia of rhesus monkeys. Other instances of the use of adenoviruses in gene therapy can be found in Rosenfeld et al., 1991, Science 252:431-434; Rosenfeld et al., 1992, Cell 68:143-155; Mastrangeli et al., 1993, J. Clin. Invest. 91:225-234; PCT Publication W094/12649; and Wang et al., 1995, Gene Therapy 2:775-783. In a preferred embodiment, adenovirus vectors are used.

Adeno-associated virus (AAV) has also been proposed for use in gene therapy (Walsh et al., 1993, Proc. Soc. Exp. Biol. Med. 204:289-300; and U.S. Pat. No. 5,436,146).

Another approach to gene therapy involves transferring a gene to cells in tissue culture, ex vivo, by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a subject. In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr, 1993, Meth. Enzymol. 217:599-618; Cohen et al., 1993, Meth. Enzymol. 217:618-644; Clin. Pharma. Ther. 29:69-92 (1985)) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a subject by various methods known in the art. Recombinant blood cells (e.g., hematopoietic stem or progenitor cells) are preferably administered intravenously. The amount of cells envisioned for use depends on the several factors including, but not limited to, the desired effects and the patient state, and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as T lymphocytes, B lymphocytes, monocytes, macrophages, neutrophils, eosinophils, mast cells, megakaryocytes, granulocytes; various stem or progenitor cells, in particular hematopoietic stem or progenitor cells (e.g., as obtained from bone marrow, umbilical cord blood, peripheral blood, fetal liver, etc.). In a preferred embodiment, the cell used for gene therapy is autologous to the subject.

In an embodiment in which recombinant cells are used in gene therapy, nucleic acid sequences encoding an antibody or fragment thereof are introduced into the cells such that they are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the present invention (see e.g., PCT Publication WO 94/08598; Stemple and Anderson, 1992, Cell 7 1:973-985; Rheinwald, 1980, Meth. Cell Bio. 21A:229; and Pittelkow and Scott, 1986, Mayo Clinic Proc. 61:771).

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

5.5 Dosage & Frequency of Administration

The amount of a prophylactic or therapeutic agent or a composition of the invention which will be effective in the prevention, treatment, management, and/or amelioration of a disorder associated with an infection with mammalian metapneumovirus can be determined by standard clinical methods. The frequency and dosage will vary also according to factors specific for each patient depending on the specific therapies (e.g., the specific therapeutic or prophylactic agent or agents) administered, the severity of the disorder, disease, or condition, the route of administration, as well as age, body, weight, response, and the past medical history of the patient. For example, the dosage of a prophylactic or therapeutic agent or a composition of the invention which will be effective in the treatment, prevention, management, and/or amelioration of a disorder associated with an infection with mammalian metapneumovirus can be determined by administering the composition to an animal model such as, e.g., the animal models disclosed herein or known in to those skilled in the art. In addition, in vitro assays may optionally be employed to help identify optimal dosage ranges. Suitable regimens can be selected by one skilled in the art by considering such factors and by following, for example, dosages are reported in literature and recommended in the Physician's Desk Reference (57th ed., 2003).

For antibodies, proteins, polypeptides, peptides and fusion proteins encompassed by the invention, the dosage administered to a patient is typically 0.0001 mg/kg to 100 mg/kg of the patient's body weight. Preferably, the dosage administered to a patient is between 0.0001 mg/kg and 20 mg/kg, 0.0001 mg/kg and 10 mg/kg, 0.0001 mg/kg and 5 mg/kg, 0.0001 and 2 mg/kg, 0.0001 and 1 mg/kg, 0.0001 mg/kg and 0.75 mg/kg, 0.0001 mg/kg and 0.5 mg/kg, 0.0001 mg/kg to 0.25 mg/kg, 0.0001 to 0.15 mg/kg, 0.0001 to 0.10 mg/kg, 0.001 to 0.5 mg/kg, 0.01 to 0.25 mg/kg or 0.01 to 0.10 mg/kg of the patient's body weight. Generally, human antibodies have a longer half-life within the human body than antibodies from other species due to the immune response to the foreign polypeptides. Thus, lower dosages of human antibodies and less frequent administration is often possible if the subject is a human. Further, the dosage and frequency of administration of antibodies of the invention or fragments thereof may be reduced by enhancing uptake and tissue penetration of the antibodies by modifications such as, for example, lipidation.

In a specific embodiment, the dosage administered to a patient will be calculated using the patient's weight in kilograms (kg) multiplied by the dose to be administered in mg/kg. The required volume (in mL) to be given is then determined by taking the mg dose required divided by the concentration of the antibody or fragment thereof in the formulations (100 mg/mL). The final calculated required volume will be obtained by pooling the contents of as many vials as are necessary into syringe(s) to administer the drug. A maximum volume of 2.0 mL of antibody or fragment thereof in the formulations can be injected per site.

In a specific embodiment, the dosage of antibodies, compositions, or combination therapies of the invention administered to prevent, treat, manage, and/or ameliorate a disorder associated with an infection with mammalian metapneumovirus in a patient is 150 μg/kg or less, preferably 125 μg/kg or less, 100 μg/kg or less, 95 μg/kg or less, 90 μg/kg or less, 85 μg/kg or less, 80 μg/kg or less, 75 μg/kg or less, 70 μg/kg or less, 65 μg/kg or less, 60 μg/kg or less, 55 μg/kg or less, 50 μg/kg or less, 45 μg/kg or less, 40 μg/kg or less, 35 μg/kg or less, 30 μg/kg or less, 25 μg/kg or less, 20 μg/kg or less, 15 μg/kg or less, 10 μg/kg or less, 5 μg/kg or less, 2.5 μg/kg or less, 2 μg/kg or less, 1.5 μg/kg or less, 1 μg/kg or less, 0.5 μg/kg or less, or 0.5 μg/kg or less of a patient's body weight. In another embodiment, the dosage of the antibodies, compositions, or combination therapies of the invention administered to prevent, treat, manage, and/or ameliorate a disorder associated with an infection with mammalian metapneumovirus, or one or more symptoms thereof in a patient is a unit dose of 0.1 mg to 20 mg, 0.1 mg to 15 mg, 0.1 mg to 12 mg, 0.1 mg to 10 mg, 0.1 mg to 8 mg, 0.1 mg to 7 mg, 0.1 mg to 5 mg, 0.1 to 2.5 mg, 0.25 mg to 20 mg, 0.25 to 15 mg, 0.25 to 12 mg, 0.25 to 10 mg, 0.25 to 8 mg, 0.25 mg to 7 mg, 0.25 mg to 5 mg, 0.5 mg to 2.5 mg, 1 mg to 20 mg, 1 mg to 15 mg, 1 mg to 12 mg, 1 mg to 10 mg, 1 mg to 8 mg, 1 mg to 7 mg, 1 mg to 5 mg, or 1 mg to 2.5 mg.

In certain embodiments, a subject is administered one or more doses of an effective amount of one or more antibodies, compositions, or combination therapies of the invention, wherein the an effective amount of said antibodies, compositions, or combination therapies prevents at least 20% to 25%, preferably at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, at least 85%, at least 90%, at least 95%, or between 95% and 100% of mammalian metapneumovirus in the subject from infecting additional cells of the subject.

In other embodiments, a subject is administered one or more does of an effective amount of one or more antibodies of the invention, wherein the dose of an effective amount achieves a serum titer of at least 0.1 μg/ml, at least 0.5 μg/ml, at least 1 μg/ml, at least 2 μg/ml, at least 5 μg/ml, at least 6 μg/ml, at least 10 μg/ml, at least 15 μg/ml, at least 20 μg/ml, at least 25 μg/ml, at least 50 μg/ml, at least 100 μg/ml, at least 125 μg/ml, at least 150 μg/ml, at least 175 μg/ml, at least 200 μg/ml, at least 225 μg/ml, at least 250 μg/ml, at least 275 μg/ml, at least 300 μg/ml, at least 325 μg/ml, at least 350 μg/ml, at least 375 μg/ml, or at least 400 μg/ml of the antibodies of the invention. In yet other embodiments, a subject is administered a dose of an effective amount of one or more antibodies of the invention to achieve a serum titer of at least 0.1 μg/ml, at least 0.5 μg/ml, at least 1 μg/ml, at least, 2 μg/ml, at least 5 μg/ml, at least 6 μg/ml, at least 10 μg/ml, at least 15 μg/ml, at least 20 μg/ml, at least 25 μg/ml, at least 50 μg/ml, at least 100 μg/ml, at least 125 μg/ml, at least 150 μg/ml, at least 175 μg/ml, at least 200 μg/ml, at least 225 μg/ml, at least 250 μg/ml, at least 275 μg/ml, at least 300 μg/ml, at least 325 μg/ml, at least 350 μg/ml, at least 375 μg/ml, or at least 400 μg/ml of the antibodies and a subsequent dose of an effective amount of one or more antibodies of the invention is administered to maintain a serum titer of at least 0.1 μg/ml, 0.5 μg/ml, 1 μg/ml, at least, 2 μg/ml, at least 5 μg/ml, at least 6 μg/ml, at least 10 μg/ml, at least 15 μg/ml, at least 20 μg/ml, at least 25 μg/ml, at least 50 μg/ml, at least 100 μg/ml, at least 125 μg/ml, at least 150 μg/ml, at least 175 μg/ml, at least 200 μg/ml, at least 225 μg/ml, at least 250 μg/ml, at least 275 μg/ml, at least 300 μg/ml, at least 325 μg/ml, at least 350 μg/ml, at least 375 μg/ml, or at least 400 μg/ml. In accordance with these embodiments, a subject may be administered 1, 2, 3, 4, 5, 6, 7, 8, 9, 10, 11, 12 or more subsequent doses.

In a specific embodiment, the invention provides methods of preventing, treating, managing, or treating a disease or disorder associated with or characterized by an infection with a mammalian metapneumovirus or one or more symptoms thereof, said method comprising administering to a subject in need thereof a dose of at least 10 μg, at least 15 μg, at least 20 μg, at least 25 μg, at least 30 μg, at least 35 μg, at least 40 μg, at least 45 μg, at least 50 μg, at least 55 μg, at least 60 μg, at least 65 μg, at least 70 μg, at least 75 μg, at least 80 μg, at least 85 μg, at least 90 μg, at least 95 μg, at least 100 μg, at least 105 μg, at least 110 μg, at least 115 μg, or at least 120 μg of one or more antibodies, combination therapies, or compositions of the invention. In certain embodiments, a dose of the antibodies of the invention may be administered once every 3 days, once every 4 days, once every 5 days, once every 6 days, once every 7 days, once every 8 days, once every 10 days, once every two weeks, once every three weeks, or once a month.

The present invention provides methods of preventing, treating, managing, or preventing a disease or disorder associated with or characterized by an infection with mammalian metapneumovirus or one or more symptoms thereof, said method comprising: (a) administering to a subject in need thereof one or more doses of a prophylactically or therapeutically effective amount of one or more antibodies, combination therapies, or compositions of the invention; and (b) monitoring the plasma level/concentration of the said administered antibody or antibodies in said subject after administration of a certain number of doses of the said antibody or antibodies. Moreover, preferably, said certain number of doses is 1, 2, 3, 4, 5, 6, 7, 8, 9, 10, 11, or 12 doses of a prophylactically or therapeutically effective amount one or more antibodies, compositions, or combination therapies of the invention. If the plasma levels of the antibody fall below a threshold level, the administration schedule can be accelerated, such that antibodies of the invention are administered more frequently.

In various embodiments, the antibodies of the invention and any second therapy (e.g., to treat or prevent an infection other than an infection with mammalian metapneumovirus) are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In preferred embodiments, two or more therapies are administered within the same patient visit.

In certain embodiments, one or more antibodies of the invention and one or more other therapies (e.g., prophylactic or therapeutic agents) are cyclically administered. Cycling therapy involves the administration of a first therapy (e.g., a first prophylactic or therapeutic agent) for a period of time, followed by the administration of a second therapy (e.g., a second prophylactic or therapeutic agent) for a period of time, optionally, followed by the administration of a third therapy (e.g., prophylactic or therapeutic agent) for a period of time and so forth, and repeating this sequential administration, i.e., the cycle in order to reduce the development of resistance to one of the therapies, to avoid or reduce the side effects of one of the therapies, and/or to improve the efficacy of the therapies.

In certain embodiments, the administration of the same antibodies of the invention may be repeated and the administrations may be separated by at least 1 day, 2 days, 3 days, 5 days, 10 days, 15 days, 30 days, 45 days, 2 months, 75 days, 3 months, or at least 6 months. In other embodiments, the administration of the same therapy (e.g., prophylactic or therapeutic agent) other than an antibody of the invention may be repeated and the administration may be separated by at least at least 1 day, 2 days, 3 days, 5 days, 10 days, 15 days, 30 days, 45 days, 2 months, 75 days, 3 months, or at least 6 months.

5.6 Biological Assays

5.6.1 Immunospecificity of Antibodies of the Invention

Antibodies of the present invention or fragments thereof may be characterized in a variety of ways well-known to one of skill in the art. In particular, antibodies of the invention or fragments thereof may be assayed for the ability to immunospecifically bind to an F protein of a mammalian metapneumovirus. Such an assay may be performed in solution (e.g., Houghten, 1992, Bio/Techniques 13:412 421), on beads (Lam, 1991, Nature 354:82 84), on chips (Fodor, 1993, Nature 364:555 556), on bacteria (U.S. Pat. No. 5,223,409), on spores (U.S. Pat. Nos. 5,571,698; 5,403,484; and 5,223,409), on plasmids (Cull et al., 1992, Proc. Natl. Acad. Sci. USA 89:1865 1869) or on phage (Scott and Smith, 1990, Science 249:386 390; Cwirla et al., 1990, Proc. Natl. Acad. Sci. USA 87:6378 6382; and Felici, 1991, J. Mol. Biol. 222:301 310) (each of these references is incorporated herein in its entirety by reference). Antibodies or fragments thereof that have been identified to immunospecifically bind to an F protein of a mammalian metapneumovirus can then be assayed for their specificity and affinity for an F protein of a mammalian metapneumovirus.

The antibodies of the invention or fragments thereof may be assayed for immunospecific binding to an F protein of a mammalian metapneumovirus and cross-reactivity with other antigens by any method known in the art. Immunoassays which can be used to analyze immunospecific binding and cross-reactivity include, but are not limited to, competitive and non-competitive assay systems using techniques such as Western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, competitive binding assays, BIAcore kinetic analysis to name but a few. Such assays are routine and well known in the art (see, e.g., Ausubel et al., eds., 1994, Current Protocols in Molecular Biology, Vol. 1, John Wiley & Sons, Inc., New York, which is incorporated by reference herein in its entirety).

The antibodies of the invention or fragments thereof can also be assayed for their ability to inhibit the fusion of a mammalian metapneumovirus to a host cell using techniques known to those of skill in the art. The antibodies of the invention or fragments thereof can also be assayed for their ability to inhibit the infection by a mammalian metapneumovirus of a host cell using techniques known to those of skill in the art.

5.6.2 In Vitro and In Vivo Assays

The antibodies, compositions, or combination therapies of the invention can be tested in vitro and/or in vivo for their ability to protect a subject from infection with mammalian metapneumovirus, to reduce the titer of mammalian metapneumovirus in a subject, or to inhibit the increase of mammalian metapneumovirus titer in a subject. Animal models can be used to assess the efficacy of an antibody, a composition, or a combination therapy of the invention.

The ability of an antibody of the invention to prevent infection, replication, propagation, and/or assembly of or by the virus can be tested in a cell culture system. In an illustrative embodiment, Vero cells are used as described in section 6.1 below.

In a specific embodiment, hamsters are administered an antibody of the invention, a composition, or a combination therapy according to the methods of the invention, challenged with mammalian metapneumovirus, and four or more days later the hamsters are sacrificed and the titer of mammalian metapneumovirus and anti-mammalian metapneumovirus antibody serum titer are determined. Further, in accordance with this embodiment, the tissues (e.g., the lung tissues) from the sacrificed hamsters can be examined for histological changes. In other embodiments, cotton rats are used for the in vivo assay.

The antibodies, compositions, or combination therapies of the invention can be tested for their ability to decrease the time course of viral infection. The antibodies, compositions, or combination therapies of the invention can also be tested for their ability to increase the survival period of humans suffering from a viral infection by at least 25%, preferably at least 50%, at least 60%, at least 75%, at least 85%, at least 95%, or at least 99%. Further, antibodies, compositions, or combination therapies of the invention can be tested for their ability reduce the hospitalization period of humans suffering from viral infection by at least 60%, preferably at least 75%, at least 85%, at least 95%, or at least 99%. Techniques known to those of skill in the art can be used to analyze the function of the antibodies, compositions, or combination therapies of the invention in vivo.

Different animal model systems can be used for different aspects of a therapy or prevention using the antibodies of the invention. Different animal model systems that can be used with the methods of the invention are described in Schmidt et al., 2004, Virus Research 106:1-13.

A number of assays may be employed in order to determine the effect of an antibody of the invention on rate of growth of a mammalian metapneumovirus in a cell culture system, an animal model system or in a subject.

The assays described herein may be used to assay viral titre over time to determine the effect of an antibody of the invention on the growth characteristics of the virus. In a specific embodiment, the viral titre is determined by obtaining a sample from the infected cells or the infected subject, preparing a serial dilution of the sample and infecting a monolayer of cells that are susceptible to infection with the virus at a dilution of the virus that allows for the emergence of infected foci that can be stained for viral protein expression. The foci can then be counted and the viral titre express as plaque forming units per milliliter of sample. In a preferred embodiment, the growth rate of a mammalian metapneumovirus in animals or humans is best tested by sampling biological fluids of a host at multiple time points post-infection and measuring viral titer.

Samples from a subject can be obtained by any method known to the skilled artisan. In certain embodiments, the sample consists of nasal aspirate, throat swab, sputum or broncho-alveolar lavage.

In certain embodiments, the determination of viral titers in cell culture or in a subject can be facilitated by testing a recombinant mammalian metapneumovirus that expresses a marker gene. In an illustrative embodiment, a mammalian metapneumovirus expresses a fluorescent protein that can be detected in cells or in animals. The effect of an antibody of the invention on the virus titers in cell culture or in animal systems can then be tested by measuring the intensity of the fluorescence in the cells or animals. A reduction in the presence of an antibody of the invention indicates that the antibody is effective at neutralizing the mammalian metapneumovirus.

Measurement of Incidence of Infection Rate

The incidence of infection can be determined by any method well-known in the art, for example, but not limited to, clinical samples (e.g., nasal swabs) can be tested for the titer of a mammalian metapneumovirus by immunofluorescence assay (IFA) to test the effectiveness of an antibody of the invention against infection with mammalian metapneumovirus.

In certain embodiments, samples containing intact cells can be directly processed, whereas isolates without intact cells should first be cultured on a permissive cell line (e.g. Vero cells or LLC-MK2 cells). In an illustrative embodiments, cultured cell suspensions should be cleared by centrifugation at, e.g., 300×g for 5 minutes at room temperature, followed by a PBS, pH 7.4 (Ca++ and Mg++ free) wash under the same conditions. Cell pellets are resuspended in a small volume of PBS for analysis. Primary clinical isolates containing intact cells are mixed with PBS and centrifuged at 300×g for 5 minutes at room temperature. Mucus is removed from the interface with a sterile pipette tip and cell pellets are washed once more with PBS under the same conditions. Pellets are then resuspended in a small volume of PBS for analysis. Five to ten microliters of each cell suspension are spotted per 5 mm well on acetone washed 12-well HTC supercured glass slides and allowed to air dry. Slides are fixed in cold (−20° C.) acetone for 10 minutes. Reactions are blocked by adding PBS—1% BSA to each well followed by a 10 minute incubation at room temperature. Slides are washed three times in PBS—0.1% Tween-20 and air dried. Ten microliters of each primary antibody reagent diluted to 250 ng/ml in blocking buffer is spotted per well and reactions are incubated in a humidified 37° C. environment for 30 minutes. Slides are then washed extensively in three changes of PBS—0.1% Tween-20 and air dried. Ten microliters of appropriate secondary conjugated antibody reagent diluted to 250 ng/ml in blocking buffer are spotted per respective well and reactions are incubated in a humidified 37° C. environment for an additional 30 minutes. Slides are then washed in three changes of PBS—0.1% Tween-20. Five microliters of PBS-50% glycerol-10 mM Tris pH 8.0-1 mM EDTA are spotted per reaction well, and slides are mounted with cover slips. Each reaction well is subsequently analyzed by fluorescence microscopy at 200× power using a B-2A filter (EX 450-490 nm). Positive reactions are scored against an autofluorescent background obtained from unstained cells or cells stained with secondary reagent alone. Positive reactions are characterized by bright fluorescence punctuated with small inclusions in the cytoplasm of infected cells.

Measurement of Serum Titer

To determine the half-life of an antibody of the invention in a biological system, the serum titer of an antibody of the invention can be determined by any method well-known in the art, for example, but not limited to, the amount of antibody or antibody fragment in serum samples can be quantitated by a sandwich ELISA. Briefly, the ELISA consists of coating microtiter plates overnight at 4° C. with an antibody that recognizes the antibody or antibody fragment in the serum. The plates are then blocked for approximately 30 minutes at room temperature with PBS-Tween-0.5% BSA. Standard curves are constructed using purified antibody or antibody fragment diluted in PBS-TWEEN-BSA, and samples are diluted in PBS-BSA. The samples and standards are added to duplicate wells of the assay plate and are incubated for approximately 1 hour at room temperature. Next, the non-bound antibody is washed away with PBS-TWEEN and the bound antibody is treated with a labeled secondary antibody (e.g., horseradish peroxidase conjugated goat-anti-human IgG) for approximately 1 hour at room temperature. Binding of the labeled antibody is detected by adding a chromogenic substrate specific for the label and measuring the rate of substrate turnover, e.g., by a spectrophotometer. The concentration of antibody or antibody fragment levels in the serum is determined by comparison of the rate of substrate turnover for the samples to the rate of substrate turnover for the standard curve at a certain dilution.

In an illustrative embodiment to determine the serum concentration of an antibody of the invention, the F protein of a mammalian MPV is linked to a solid support. Subsequently, the material that is to be tested for the concentration of the antibody of the invention is incubated with the solid support under conditions conducive to the binding of the antibodies to the mammalian MPV components. Subsequently, the solid support is washed under conditions that remove any unspecifically bound antibodies. Following the washing step, the presence of bound antibodies can be detected using any technique known to the skilled artisan. In a specific embodiment, the mammalian MPV protein-antibody complex is incubated with detectably labeled antibody that recognizes the antibody of the invention under conditions conducive to the binding of the detectably labeled antibody to the antibody that is bound to the component of mammalian MPV. In a specific embodiment, the detectably labeled antibody is conjugated to an enzymatic activity. In another embodiment, the detectably labeled antibody is radioactively labeled. The complex of mammalian MPV protein-antibody-detectably labeled antibody is then washed, and subsequently the presence of the detectably labeled antibody is quantified by any technique known to the skilled artisan, wherein the technique used is dependent on the type of label of the detectably labeled antibody.

Biacore Assay

Determination of the kinetic parameters of antibody binding can be determined for example by the injection of 250 μL of monoclonal antibody (“mAb”) at varying concentration in HBS buffer containing 0.05% Tween-20 over a sensor chip surface, onto which has been immobilized the antigen. The antigen can be the F protein of a mammalian MPV. The flow rate is maintained constant at 75 uL/min. Dissociation data is collected for 15 min, or longer as necessary. Following each injection/dissociation cycle, the bound mAb is removed from the antigen surface using brief, 1 min pulses of dilute acid, typically 10-100 mM HCl, though other regenerants are employed as the circumstances warrant.

More specifically, for measurement of the rates of association, kₒₙ, and dissociation, k_{off}, the antigen is directly immobilized onto the sensor chip surface through the use of standard amine coupling chemistries, namely the EDC/NHS method (EDC=N-diethylaminopropyl)-carbodiimide). Briefly, a 5-100 nM solution of the antigen in 10 mM NaOAc, pH4 or pH5 is prepared and passed over the EDC/NHS-activated surface until approximately 30-50 RU's (Biacore Resonance Unit) worth of antigen are immobilized. Following this, the unreacted active esters are “capped” off with an injection of 1M Et-NH2. A blank surface, containing no antigen, is prepared under identical immobilization conditions for reference purposes. Once a suitable surface has been prepared, an appropriate dilution series of each one of the antibody reagents is prepared in HBS/Tween-20, and passed over both the antigen and reference cell surfaces, which are connected in series. The range of antibody concentrations that are prepared varies depending on what the equilibrium binding constant, K_{D}, is estimated to be. As described above, the bound antibody is removed after each injection/dissociation cycle using an appropriate regenerant.

Once an entire data set is collected, the resulting binding curves are globally fitted using algorithms supplied by the instrument manufacturer, BIAcore, Inc. (Piscataway, N.J.). All data are fitted to a 1:1 Langmuir binding model. These algorithm calculate both the kₒₙ and the k_{off}, from which the apparent equilibrium binding constant, K_{D}, is deduced as the ratio of the two rate constants (i.e. k_{off}/kₒₙ). More detailed treatments of how the individual rate constants are derived can be found in the BIAevaluation Software Handbook (BIAcore, Inc., Piscataway, N.J.).

Microneutralization Assay

The ability of antibodies of the invention or antigen-binding fragments thereof to neutralize virus infectivity is determined by a microneutralization assay. This microneutralization assay is a modification of the procedures described by Anderson et al., (1985, J. Clin. Microbiol. 22:1050-1052, the disclosure of which is hereby incorporated by reference in its entirety). The procedure is also described in Johnson et al., 1999, J. Infectious Diseases 180:35-40, the disclosure of which is hereby incorporated by reference in its entirety.

Antibody dilutions are made in triplicate using a 96-well plate. 50 to 1000 TCID₅₀ of a mammalian MPV are incubated with serial dilutions of the antibody or antigen-binding fragments thereof to be tested for 2 hours at 37° C. in the wells of a 96-well plate. Virus mixtures are added to 80-95% confluent cells susceptible to infection with a mammalian MPV, such as, but not limited to Vero cells and cultured for 5 days at 37° C. in 5% CO₂. After 5 days, the medium is aspirated and cells are washed and fixed to the plates with 80% acetone and 20% PBS. Virus replication is then determined by viral antigen, such as F protein expression. Fixed cells are incubated with a biotin-conjugated anti-viral antigen, such as anti-F protein monoclonal antibody washed and horseradish peroxidase conjugated avidin is added to the wells. The wells are washed again and turnover of substrate TMB (thionitrobenzoic acid) is measured at 450 nm. The neutralizing titer is expressed as the antibody concentration that causes at least 50% reduction in absorbency at 450 nm (the OD₄₅₀) from virus-only control cells.

The microneutralization assay described here is only one example. Alternatively, standard neutralization assays can be used to determine how significantly the virus is affected by an antibody.

Viral Fusion Inhibition Assay

In certain embodiments, a viral fusion inhibition assay may be used. This assay is in principle identical to the microneutralization assay, except that the cells are infected with the respective virus for four hours prior to addition of antibody and the read-out is in terms of presence of absence of fusion of cells (Taylor et al., 1992, J. Gen. Virol. 73:2217-2223).

Challenge Studies

This assay is used to determine the ability of an antibody of the inveniton to prevent infection with mammalian metapneumovirus in an animal model system, such as, but not limited to, cotton rats or hamsters. The antibody of the invention can be administered by intravenous (IV) route, by intramuscular (IM) route or by intranasal route (IN). Infection can occur by any technique well-known to the skilled artisan. This assay is also used to correlate the serum concentration of antibodies with a reduction in lung titer of the virus to which the antibodies bind.

On day 0, groups of animals, such as, but not limited to, cotton rats (Sigmodon hispidis, average weight 100 g) cynomolgous macacques (average weight 2.0 kg) are administered the antibody of the invention or BSA by intramuscular injection, by intravenous injection, or by intranasal route. Prior to, concurrently with, or subsequent to administration of the antibody of the invention, the animals are infected with wild type virus wherein the wild type mammalian metapneumovirus. In certain embodiments, the animals are infected with the wild type virus at least 1 day, at least 2 days, at least 3 days, at least 4 days, at least 5 days, at least 6 days, 1 week or 1 or more months subsequent to the administration of the antibody of the invention.

After the infection, cotton rats are sacrificed, and their lung tissue is harvested and pulmonary virus titers are determined by titration of infected foci. Bovine serum albumin (BSA) 10 mg/kg is used as a negative control. Antibody concentrations in the serum at the time of challenge are determined using a sandwich ELISA. Similarly, in macacques, virus titers in nasal and lung lavages can be measured.

5.6.3 Toxicity Assays

The toxicity and/or efficacy of the prophylactic and/or therapeutic protocols of the instant invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Therapies that exhibit large therapeutic indices are preferred. While therapies that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage of the prophylactic and/or therapeutic agents for use in humans. The dosage of such agents lies preferably within a range of circulating concentrations that include the ED₅₀ with little or no toxicity. The dosage may vary within this range depending upon the dosage form employed and the route of administration utilized. For any therapy used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

Further, any assays known to those skilled in the art can be used to evaluate the prophylactic and/or therapeutic utility of an antibody, a composition, a combination therapy disclosed herein for a disease or disorder associated with or characterized by an infection with mammalian metapneumovirus or one or more symptoms thereof.

5.7 Diagnostic Uses of Antibodies

Antibodies of the invention (including molecules comprising, or alternatively consisting of, antibody fragments or variants thereof) that immunospecifically bind to a an F protein of a mammalian metapneumovirus can be used for diagnostic purposes to detect an infection with mammalian metapneumovirus.

In certain embodiments, an antibody of the invention is conjugated with a detectable label to facilitate the deteciton of a mammalian metapneumovirus.

Antibodies of the invention can be used to assay mammalian metapneumovirus titers in a biological sample using classical immunohistological methods as known to those of skill in the art (e.g., see Jalkanen et al., 1985, J. Cell. Biol. 101:976-985; and Jalkanen et al., 1987, J. Cell. Biol. 105:3087-3096). Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase; radioisotopes, such as iodine (125I, 121I), carbon (14C), sulfur (35S), tritium (3H), indium (121In), and technetium (99Tc); luminescent labels, such as luminol; and fluorescent labels, such as fluorescein and rhodamine, and biotin.

In certain embodiments, the antibody of the invention binds to the F protein of all subtypes of mammalian metapneumovirus, i.e., subtype A1, A2, B1, and B2. In certain embodiments, an antibody binds specifically to a subtype of human metapneumovirus, i.e., subtype A1, A2, B1, and B2. Thus, depending on the specificity of the antibody, infection with a specific subtype of mammalian metapneumovirus can be diagnosed.

Without being bound by theory, the methods of the invention are particularly useful where the symptoms of a subject do not allow an unambigous diagnosis.

In certain embodiments, antibodies of the invention can also be used to diagnose infections with avian pneumovirus in birds.

5.8 Kits

The present invention provides kits that can be used in the above methods. In one embodiment, a kit comprises an antibody of the invention, preferably a purified antibody, in one or more containers. In another embodiment, a kit comprises an antibody fragment of the invention that immunospecifically binds to an F protein of a mammalian metapneumovirus. In certain embodiments, the kits of the present invention further comprise a control antibody which does not react with an F protein of a mammalian metapneumovirus. In another specific embodiment, the kits of the present invention contain a means for detecting the binding of an antibody to an F protein of a mammalian metapneumovirus (e.g., the antibody may be conjugated to a detectable substrate such as a fluorescent compound, an enzymatic substrate, a radioactive compound or a luminescent compound, a second antibody which recognizes the first antibody may be conjugated to a detectable substrate, or the antibody may be bound to a solid surface such that binding of the F protein or the whole virus results in a change in the optical properties of the solid surface).

In an additional embodiment, the invention provides a diagnostic kit for use in screening serum containing an F protein of a mammalian metapneumovirus. The diagnostic kit includes a substantially isolated antibody of the invention, and means for detecting the binding of the F protein of a mammalian metapneumovirus to the antibody. In one embodiment, the antibody is attached to a solid support. In a specific embodiment, the antibody may be a monoclonal antibody. The detecting means of the kit may include a second, labeled monoclonal antibody. Alternatively, or in addition, the detecting means may include a labeled, competing antigen.

5.9 Articles of Manufacture

The present invention also encompasses a finished packaged and labeled pharmaceutical product. This article of manufacture includes the appropriate unit dosage form in an appropriate vessel or container such as a glass vial or other container that is hermetically sealed. The pharmaceuctical product may be formulated in single dose vials as a sterile liquid that contains 10 mM histidine buffer at pH 6.0 and 150 mM sodium chloride. Each 1.0 mL of solution may contain 100 mg of protein, 1.6 mg of histidine and 8.9 mg of sodium chloride in water for injection. During the manufacturing process the pH of the formulation buffer is adjusted to 6.0 using hydrochloric acid. In the case of dosage forms suitable for parenteral administration the active ingredient, e.g., an antibody of the invention that immunospecifically binds to an F protein of a mammalian metapneumovirus, is sterile and suitable for administration as a particulate free solution. In other words, the invention encompasses both parenteral solutions and lyophilized powders, each being sterile, and the latter being suitable for reconstitution prior to injection. Alternatively, the unit dosage form may be a solid suitable for oral, transdermal, intransal, or topical delivery.

In a preferred embodiment, the unit dosage form is suitable for intravenous, intramuscular, intranasal, oral, topical or subcutaneous delivery. Thus, the invention encompasses solutions, preferably sterile, suitable for each delivery route.

As with any pharmaceutical product, the packaging material and container are designed to protect the stability of the product during storage and shipment. Further, the products of the invention include instructions for use or other informational material that advise the physician, technician or patient on how to appropriately prevent or treat the disease or disorder in question. In other words, the article of manufacture includes instruction means indicating or suggesting a dosing regimen including, but not limited to, actual doses and monitoring procedures.

Specifically, the invention provides an article of manufacture comprising packaging material, such as a box, bottle, tube, vial, container, sprayer, insufflator, intravenous (i.v.) bag, envelope and the like; and at least one unit dosage form of a pharmaceutical agent contained within said packaging material, wherein said pharmaceutical agent comprises an antibody that immunospecifically binds an F protein of a mammalian metapneumovirus and wherein said packaging material includes instruction means which indicate that said antibody can be used to prevent, manage, treat, and/or ameliorate one or more symptoms associated with a disorder associated with an infection with mammalian metapneumovirus, or one or more symptoms thereof by administering specific doses and using specific dosing regimens as described herein.

5.10 Methods of Producing Antibodies

Antibodies that immunospecifically bind to an antigen can be produced by any method known in the art for the synthesis of antibodies, in particular, by chemical synthesis or preferably, by recombinant expression techniques.

Polyclonal antibodies that immunospecifically bind to an antigen can be produced by various procedures well-known in the art. For example, a human antigen can be administered to various host animals including, but not limited to, rabbits, mice, rats, etc. to induce the production of sera containing polyclonal antibodies specific for the human antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T Cell Hybridomas 563 681 (Elsevier, N.Y., 1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. Briefly, mice can be immunized with an F protein of a mammalian metapneumovirus and once an immune response is detected, e.g., antibodies specific for an F protein of a mammalian metapneumovirus are detected in the mouse serum, the mouse spleen is harvested and splenocytes isolated. The splenocytes are then fused by well known techniques to any suitable myeloma cells, for example cells from cell line SP20 available from the ATCC. Hybridomas are selected and cloned by limited dilution. Additionally, a RIMMS (repetitive immunization multiple sites) technique can be used to immunize an animal (Kilptrack et al., 1997 Hybridoma 16:381-9, incorporated by reference in its entirety). The hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

Accordingly, the present invention provides methods of generating antibodies by culturing a hybridoma cell secreting an antibody of the invention wherein, preferably, the hybridoma is generated by fusing splenocytes isolated from a mouse immunized with an F protein of a mammalian metapneumovirus with myeloma cells and then screening the hybridomas resulting from the fusion for hybridoma clones that secrete an antibody able to bind to an F protein of a mammalian metapneumovirus.

Antibody fragments that immunospecifically bind to an F protein of a mammalian metapneumovirus may be generated by any technique known to those of skill in the art. For example, Fab and F(ab′)₂ fragments of the invention may be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the CH1 domain of the heavy chain. Further, the antibodies of the present invention can also be generated using various phage display methods known in the art.

In phage display methods, functional antibody domains are displayed on the surface of phage particles which carry the polynucleotide sequences encoding them. In particular, DNA sequences encoding VH and VL domains are amplified from animal cDNA libraries (e.g., human or murine cDNA libraries of affected tissues). The DNA encoding the VH and VL domains are recombined together with an scFv linker by PCR and cloned into a phagemid vector. The vector is electroporated in E. coli and the E. coli is infected with helper phage. Phage used in these methods are typically filamentous phage including fd and M13 and the VH and VL domains are usually recombinantly fused to either the phage gene III or gene VIII. Phage expressing an antigen binding domain that binds to a particular antigen can be selected or identified with antigen, e.g., using labeled antigen or antigen bound or captured to a solid surface or bead. Examples of phage display methods that can be used to make the antibodies of the present invention include those disclosed in Brinkman et al., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology 57:191-280; PCT Application No. PCT/GB91/01134; International Publication Nos. WO 90/02809, WO 91/10737, WO 92/01047, WO 92/18619, WO 93/1 1236, WO 95/15982, WO 95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described below. Techniques to recombinantly produce Fab, Fab′ and F(ab′)₂ fragments can also be employed using methods known in the art such as those disclosed in PCT publication No. WO 92/22324; Mullinax et al., 1992, BioTechniques 12(6):864-869; Sawai et al., 1995, AJRI 34:26-34; and Better et al., 1988, Science 240:1041-1043 (said references incorporated by reference in their entireties).

To generate whole antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a VH constant region, e.g., the human gamma 4 constant region, and the PCR amplified VL domains can be cloned into vectors expressing a VL constant region, e.g., human kappa or lamba constant regions. Preferably, the vectors for expressing the VH or VL domains comprise an EF-1α promoter, a secretion signal, a cloning site for the variable domain, constant domains, and a selection marker such as neomycin. The VH and VL domains may also cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

For some uses, including in vivo use of antibodies in humans and in vitro detection assays, it may be preferable to use human or chimeric antibodies. Completely human antibodies are particularly desirable for therapeutic treatment of human subjects. Human antibodies can be made by a variety of methods known in the art including phage display methods described above using antibody libraries derived from human immunoglobulin sequences. See also U.S. Pat. Nos. 4,444,887 and 4,716,111; and International Publication Nos. WO 98/46645, WO 98/50433, WO 98/24893, WO98/16654, WO 96/34096, WO 96/33735, and WO 91/10741; each of which is incorporated herein by reference in its entirety. Humanization of antibodies can also be accomplished using the techniques taught in U.S. application Ser. No. 10/923,068 filed Aug. 20, 2004 and published as US 2005/0042664 on Feb. 24, 2005, which is incorporated herein by reference in its entirety.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then be bred to produce homozygous offspring which express human antibodies. The transgenic mice are immunized in the normal fashion with a selected antigen, e.g., all or a portion of an F protein of a mammalian metapneumovirus. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65 93). For a detailed discussion of this technology for producing human antibodies and human monoclonal antibodies and protocols for producing such antibodies, see, e.g., PCT publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598, which are incorporated by reference herein in their entirety. In addition, companies such as Abgenix, Inc. (Freemont, Calif.) and Genpharm (San Jose, Calif.) can be engaged to provide human antibodies directed against a selected antigen using technology similar to that described above.

A chimeric antibody is a molecule in which different portions of the antibody are derived from different immunoglobulin molecules. Methods for producing chimeric antibodies are known in the art. See e.g., Morrison, 1985, Science 229:1202; Oi et al., 1986, BioTechniques 4:214; Gillies et al., 1989, J. Immunol. Methods 125:191-202; and U.S. Pat. Nos. 5,807,715, 4,816,567, 4,816,397, and 6,331,415, which are incorporated herein by reference in their entirety.

A humanized antibody is an antibody or its variant or fragment thereof which is capable of binding to a predetermined antigen and which comprises a framework region having substantially the amino acid sequence of a human immunoglobulin and a CDR having substantially the amino acid sequence of an immuoglobulin that is known to bind the the antigen of interest (the “donor antibody”), e.g., an F protein of a human metapneumovirus. A humanized antibody comprises substantially all of at least one, and typically two, variable domains (Fab, Fab′, F(ab′).sub.2, Fabc, Fv) in which all or substantially all of the CDR regions correspond to those of the donor antibody and all or substantially all of the framework regions are those of a human immunoglobulin consensus sequence. Preferably, a humanized antibody also comprises at least a portion of an immunoglobulin constant region (Fc), typically that of a human immunoglobulin. Ordinarily, the antibody will contain both the light chain as well as at least the variable domain of a heavy chain. The antibody also may include the CH1, hinge, CH2, CH3, and CH4 regions of the heavy chain. The humanized antibody can be selected from any class of immunoglobulins, including IgM, IgG, IgD, IgA and IgE, and any isotype, including IgG1, IgG2, IgG3 and IgG4. Usually the constant domain is a complement fixing constant domain where it is desired that the humanized antibody exhibit cytotoxic activity, and the class is typically IgG.sub.1. Where such cytotoxic activity is not desirable, the constant domain may be of the IgG.sub.2 class. The humanized antibody may comprise sequences from more than one class or isotype, and selecting particular constant domains to optimize desired effector functions is within the ordinary skill in the art. The framework and CDR regions of a humanized antibody need not correspond precisely to the parental sequences, e.g., the donor CDR or the consensus framework may be mutagenized by substitution, insertion or deletion of at least one residue so that the CDR or framework residue at that site does not correspond to either the consensus or the import antibody. Such mutations, however, will not be extensive. Usually, at least 75% of the humanized antibody residues will correspond to those of the parental FR and CDR sequences, more often 90%, and most preferably greater than 95%. Humanized antibody can be produced using variety of techniques known in the art, including but not limited to, CDR-grafting (European Patent No. EP 239,400; International publication No. WO 91/09967; and U.S. Pat. Nos. 5,225,539, 5,530,101, and 5,585,089), veneering or resurfacing (European Patent Nos. EP 592,106 and EP 519,596; Padlan, 1991, Molecular Immunology 28(4/5):489-498; Studnicka et al., 1994, Protein Engineering 7(6):805-814; and Roguska et al., 1994, PNAS 91:969-973), chain shuffling (U.S. Pat. No. 5,565,332), and techniques disclosed in, e.g., U.S. Pat. No. 6,407,213, U.S. Pat. No. 5,766,886, WO 9317105, Tan et al., J. Immunol. 169:1119 25 (2002), Caldas et al., Protein Eng. 13(5):353 60 (2000), Morea et al., Methods 20(3):267 79 (2000), Baca et al., J. Biol. Chem. 272(16): 10678 84 (1997), Roguska et al., Protein Eng. 9(10):895 904 (1996), Couto et al., Cancer Res. 55 (23 Supp):5973s 5977s (1995), Couto et al., Cancer Res. 55(8):1717 22 (1995), Sandhu J S, Gene 150(2):409 10 (1994), and Pedersen et al., J. Mol. Biol. 235(3):959 73 (1994). Often, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature 332:323, which are incorporated herein by reference in their entireties.) Single domain antibodies, for example, antibodies lacking the light chains, can be produced by methods well-known in the art. See Riechmann et al., 1999, J. Immuno. 231:25-38; Nuttall et al., 2000, Curr. Pharm. Biotechnol. 1(3):253-263; Muylderman, 2001, J. Biotechnol. 74(4):277302; U.S. Pat. No. 6,005,079; and International Publication Nos. WO 94/04678, WO 94/25591, and WO 01/44301, each of which is incorporated herein by reference in its entirety.

Further, the antibodies that immunospecifically bind to an antigen (e.g., F protein of a mammalian metapneumovirus) can, in turn, be utilized to generate anti-idiotype antibodies that “mimic” an antigen using techniques well known to those skilled in the art. (See, e.g., Greenspan & Bona, 1989, FASEB J. 7(5):437-444; and Nissinoff, 1991, J. Immunol. 147(8):2429-2438).

5.10.1 Polynucleotide Sequences Encoding Antibodies

The invention provides polynucleotides comprising a nucleotide sequence encoding an antibody or fragment thereof that immunospecifically binds to an F protein of a mammalian metapneumovirus. The invention also encompasses polynucleotides that hybridize under high stringency, intermediate or lower stringency hybridization conditions, e.g., as defined supra, to polynucleotides that encode an antibody of the invention.

The polynucleotides may be obtained, and the nucleotide sequence of the polynucleotides determined, by any method known in the art. Since the amino acid sequences of mAb234 and mAb338 are known, nucleotide sequences encoding these antibodies can be determined using methods well known in the art, i.e., nucleotide codons known to encode particular amino acids are assembled in such a way to generate a nucleic acid that encodes the antibody. Such a polynucleotide encoding the antibody may be assembled from chemically synthesized oligonucleotides (e.g., as described in Kutmeier et al., 1994, BioTechniques 17:242), which, briefly, involves the synthesis of overlapping oligonucleotides containing portions of the sequence encoding the antibody, fragments, or variants thereof, annealing and ligating of those oligonucleotides, and then amplification of the ligated oligonucleotides by PCR.

Alternatively, a polynucleotide encoding an antibody may be generated from nucleic acid from a suitable source. If a clone containing a nucleic acid encoding a particular antibody is not available, but the sequence of the antibody molecule is known, a nucleic acid encoding the immunoglobulin may be chemically synthesized or obtained from a suitable source (e.g., an antibody cDNA library or a cDNA library generated from, or nucleic acid, preferably poly A+ RNA, isolated from, any tissue or cells expressing the antibody, such as hybridoma cells selected to express an antibody of the invention) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

Once the nucleotide sequence of the antibody is determined, the nucleotide sequence of the antibody may be manipulated using methods well known in the art for the manipulation of nucleotide sequences, e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N.Y. and Ausubel et al., eds., 1998, Current Protocols in Molecular Biology, John Wiley & Sons, NY, which are both incorporated by reference herein in their entireties), to generate antibodies having a different amino acid sequence, for example to create amino acid substitutions, deletions, and/or insertions.

In a specific embodiment, one or more of the CDRs listed in Table 1 is inserted within framework regions using routine recombinant DNA techniques. The framework regions may be naturally occurring or consensus framework regions, and preferably human framework regions (see, e.g., Chothia et al., 1998, J. Mol. Biol. 278: 457-479 for a listing of human framework regions). Preferably, the polynucleotide sequence generated by the combination of the framework regions and CDRs encodes an antibody that immunospecifically binds to an F protein of human metapneumovirus. Preferably, one or more amino acid substitutions may be made within the framework regions, and, preferably, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present invention and within the skill of the art.

5.10.2 Recombinant Expression of Antibodies

Recombinant expression of an antibody of the invention (e.g., a heavy or light chain of an antibody of the invention or a fragment thereof or a single chain antibody of the invention) that immunospecifically binds to an F protein of mammalian metapneumovirus requires construction of an expression vector containing a polynucleotide that encodes the antibody. Once a polynucleotide encoding an antibody molecule, heavy or light chain of an antibody, or fragment thereof (preferably, but not necessarily, containing the heavy or light chain variable domain) of the invention has been obtained, the vector for the production of the antibody molecule may be produced by recombinant DNA technology using techniques well-known in the art. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, a heavy or light chain of an antibody, a heavy or light chain variable domain of an antibody or a fragment thereof, or a heavy or light chain CDR, operably linked to a promoter. Such vectors may include the nucleotide sequence encoding the constant region of the antibody molecule (see, e.g., International Publication No. WO 86/05807; International Publication No. WO 89/01036; and U.S. Pat. No. 5,122,464) and the variable domain of the antibody may be cloned into such a vector for expression of the entire heavy, the entire light chain, or both the entire heavy and light chains.

The expression vector is transferred to a host cell by conventional techniques and the transfected cells are then cultured by conventional techniques to produce an antibody of the invention. Thus, the invention includes host cells containing a polynucleotide encoding an antibody of the invention or fragments thereof, or a heavy or light chain thereof, or fragment thereof, or a single chain antibody of the invention, operably linked to a heterologous promoter. In preferred embodiments for the expression of double-chained antibodies, vectors encoding both the heavy and light chains may be co-expressed in the host cell for expression of the entire immunoglobulin molecule, as detailed below.

A variety of host-expression vector systems may be utilized to express the antibody molecules of the invention (see, e.g., U.S. Pat. No. 5,807,715). Such host-expression systems represent vehicles by which the coding sequences of interest may be produced and subsequently purified, but also represent cells which may, when transformed or transfected with the appropriate nucleotide coding sequences, express an antibody molecule of the invention in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, NS0, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., 1986, Gene 45:101; and Cockett et al., 1990, Bio/Technology 8:2). In a specific embodiment, the expression of nucleotide sequences encoding antibodies of the invention, derivative, analog, or fragment thereof which immunospecifically bind to an F protein of a mammalian metapneumovirus or fragments thereof is regulated by a constitutive promoter, inducible promoter or tissue specific promoter.

In bacterial systems, a number of expression vectors may be advantageously selected depending upon the use intended for the antibody molecule being expressed. For example, when a large quantity of such an antibody is to be produced, for the generation of pharmaceutical compositions of an antibody molecule, vectors which direct the expression of high levels of fusion protein products that are readily purified may be desirable. Such vectors include, but are not limited to, the E. coli expression vector pUR278 (Ruther et al., 1983, EMBO 12:1791), in which the antibody coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors may also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts (e.g., see Logan & Shenk, 1984, Proc. Natl. Acad. Sci. USA 8 1:355-359). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., 1987, Methods in Enzymol. 153:51-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host-cells which possess the cellular machinery for proper processing of the primary transcript, glycosylation, and phosphorylation of the gene product may be used. Such mammalian host cells include but are not limited to CHO, VERY, BHK, Hela, COS, MDCK, 293, 3T3, W138, BT483, Hs578T, HTB2, BT2O and T47D, NS0 (a murine myeloma cell line that does not endogenously produce any immunoglobulin chains), CRL7O3O and HsS78Bst cells.

For long-term, high-yield production of recombinant proteins, stable expression is preferred. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the antibody molecule. Such engineered cell lines may be particularly useful in screening and evaluation of compositions that interact directly or indirectly with the antibody molecule.

A number of selection systems may be used, including but not limited to, the herpes simplex virus thymidine kinase (Wigler et al., 1977, Cell 11:223), hypoxanthineguanine phosphoribosyltransferase (Szybalska & Szybalski, 1992, Proc. Natl. Acad. Sci. USA 48:202), and adenine phosphoribosyltransferase (Lowy et al., 1980, Cell 22:8-17) genes can be employed in tk-, hgprt- or aprt-cells, respectively. Also, antimetabolite resistance can be used as the basis of selection for the following genes: dhfr, which confers resistance to methotrexate (Wigler et al., 1980, Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62: 191-217; May, 1993, TIB TECH 11(5):155-2 15); and hygro, which confers resistance to hygromycin (Santerre et al., 1984, Gene 30:147). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., 1981, J. Mol. Biol. 150: 1, which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Vol. 3. (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., 1983, Mol. Cell. Biol. 3:257).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot, 1986, Nature 322:52; and Kohler, 1980, Proc. Natl. Acad. Sci. USA 77:2 197). The coding sequences for the heavy and light chains may comprise cDNA or genomic DNA.

Once an antibody molecule of the invention has been produced by recombinant expression, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies of the present invention or fragments thereof may be fused to heterologous polypeptide sequences described herein or otherwise known in the art to facilitate purification.

Recombinant expression as described above may also be used to produce immunogens derived from an F protein of a mammalian metapneumovirus.

The present invention may be better understood by reference to the following non-limiting Examples, which are provided as exemplary of the invention. The following examples are presented in order to more fully illustrate the preferred embodiments of the invention. They should in no way be construed, however, as limiting the broad scope of the invention.

### 6. EXAMPLE

### Production of Monoclonal Antibodies with Potent in Vitro and in Vivo Neutralizing Activity Directed Against Human Metapneumovirus

6.1. Materials and Methods

Cells and Virus. Vero, WI-38, LLC-MK2 (ATCC) cells used for the propagation of hMPV and b/hPIV3 (see below) derived viruses were maintained in Eagle modified minimal essential medium (EMEM) supplemented with 10% fetal bovine serum (FBS) prior to use for viral propagation. Adenovirus vectors were grown in HEK-293 cells cultured in DMEM+10% FBS.

Prototype hMPV strains were obtained from A. Osterhaus. The prototypes were as follows: A1 NL\1\00, A2 NL\1\00, B1 NL\1\99 and B2 NL\1\94 (1). For hMPV propagation, semiconfluent cell monolayers were infected at a multiplicity of infection of 0.1 in EMEM without FBS plus 2.5 μg/ml trypsin. Virions were harvested from cells following freeze-thaw cellular disruption between days 5-9. Cellular debris was romoved by centrifugation at 1500×g and the supernatant was retained as the viral stock. Viral samples were stabilized by the addition of one tenth volume of 10× SPG (2.18 M sucrose, 0.038 M KH2PO4, 0.054 M L-glutamate). Viral titers were determined by serial dilution on Vero or LLC-MK2 cells. Viral replication was then measured using an F protein-specific ELISA. TCID₅₀ were calculated using the Karber method (5).

PIV3 vectored hMPV fusion protein virus (b/hPIV3/hMPV F) has been reported previously and was propagated as described in Vero cells (2). Virus concentrations were determined by plaque assay on Vero cells.

Adenovirus constructs expressing the fusion protein from hMPV strains NL\1\00 and NL\1\99 were produced using the AdEasy adenoviral system with the pShuttle-CMV transfer vector. The resultant adenovirus was propagated in HEK293 cells according to the manufacturers' instructions (AdEasy, Stratagene, LaJolla, Calif.). Viral titers were calculated as TCID₅₀ as determined by cytopathic effect of serial dilutions on HEK293 cells.

Production of Hybridoma Cell Lines. Armenian hamsters (Cytogen Resesarch and Development, Inc. Boston, Mass.) and BALB/c mice (Jackson Laboratory, Bar Harbor, Me.) were immunized using a combination of some or all of the following: Intranasal infection with hMPV at 10⁶ TCID₅₀ per animal of either NL\1\7\00, NL\1\00 or NL\1\99, intranasal infect with 10⁶ PFU b/hPIV2/hMPV F_{NL\1\00}, intraperitoneal adenovirus vectored hMPV F_{NL\1\00} or hMPV F_{NL\1\99} at a dose of 9×10⁷ TCID₅₀, intraperitoneal injection of purified soluble hMPV fusion protein derived from NL\1\00 and NL\1\99 with either GERBU adjuvant (C-C Biotech) at a dose of 2 microgram GMPD ((N-acetylglucosaminyl-betal-4 N-acetyl muramul-L-alanyl-D-isoglytamine) and 4 micorgram lipids (dimethyl distearolylhydroxyethyl ammonium chloride) or in an adjuvant-free solution. Four days after the final immunization, splenic lymphocytes were fused by the polyethylene glycol fusion method to NS-0 cells as described previously (3). Fusions were plated either in semi-solid medium (ClonaCell, Stem Cell Technologies, Vancouver, BC) or in liquid medium in 96 well plates. Hybridoma supernatants that produced hMPV-specific antibodies were identified by ELISA on hMPV-infected cells.

Sequencing of mouse monoclonal CDRs. RNA was isolated from hybridoma cells expressing the mouse antibodies of interest. The sequences of the mouse CDRs were amplified by PCR using commercially available probes (EMD Biosciences, La Jolla, Calif.) and were cloned into topoisomerase bound TA overhang plasmid vectors (Invitrogen). Multiple clones of the plasmid vectors were isolated and sequenced to derive a concensus sequence of the mouse hypervariable regions.

F protein construct generation. Full length, and truncated F protein lacking the transmembrane domain, were made using plasmids RF516 and RF515 from the laboratory of A. Osterhaus as the PCR template. Plasmids RF516 and RF515 are the full length sequences of the HMPV F protein from NL\1\00 and NL\1\99, respectively, cloned into plasmid, pSA91 (Virus Res. Feb. 26, 2002;83(1-2):43-56) using the HindIII and EcoR1 site of this vector for cloning hMPV Fs. To obtain a soluble histidine-tagged form of the hMPV F protein, the following oligonucleotides were used. For soluble fusion protein from NL\1\00 (sF_{N\1\00}),: 5′-aaccaaaagcttcacc ATGtcttggaaagtggtgatc-3′ (SEQ ID NO: 117) and 5′-ttaattgaattcttagtgatggtgatggtgatggccagtgtttcctttctctgc 3′ (SEQ ID NO: 118), for soluble fusion protein from NL\1\99 (sF_{NL\1\99}): 5′ttccttaagcttcaccATGTCTTGGAAAGTGATGATCATC 3′ (SEQ ID NO: 119) and 5′ ttaattggatccttagtgatggtgatggtgatgaccagtgtttcctttttctgcact 3′ (SEQ ID NO:120). The PCR products were cleaved using the restriction endonucleases EcoRI and HindIII for the NL\1\00 sequence and BamHI and HindIII for the NL\1\99 sequence and ligated to the vector pcDNA3.1(+) -digested with the same endonucleases. The pcDNA clones were transiently transfected into 293 cells using lipofectamine to introduce the DNA into the cells. To make constructs for stable expression of hMPV F protein, the same plasmid source of DNA was used to generate PCR products; however the following primers were used for both the NL1/00 and NL/1/99 sequences: 5′-aat caa cgg tcc gcc acc atg tct tgg aaa gtg-3′ (SEQ ID NO:121) and 5′ ttaattgaattcttagtgatggtgatggtgatggccagtgtttcctttctctgc 3′ (SEQ ID NO:122). The PCR products were cleaved with RsrII and EcoRI, and ligated to the pEE15.1 vector (Lonza) cleaved with the same restriction endonucleases. Stable NS—O cell lines were made as describe by Bebblington (4). Full-length F protein constructs were made using the following oligonucleotides: For NL\1\00 5′ aaccaaaagcttcacc ATGtcttggaaagtggtgatc 3′ (SEQ ID NO:123) and 5′ aattaaggatcC taattatgtggtatgaagccatT 3′ (SEQ ID NO:124) and for NL\1\99 5′ttccttaagcttcacc ATGTCTTGGAAAGTGATGATCATC 3′ (SEQ ID NO:125) and 5′ aattaaggatcC taattatgtggtatgaaaccgcc (SEQ ID NO: 126). PCR products were cleaved with BamHI and HindIII endonucleases and were ligated to pcDNA3.1(+) cleaved with the same endonucleases. These vectors were used as the source of DNA for the construction of the adenovirus transfer vector pShuttle-CMV. The full length F protein-containing fragments were obtained by cleavage of the pcDNA3.1 clones with the restriction endonucleases HindIII and EcoRV, and were ligated to the pShuttle-CMV vector cleaved with the same endonucleases.

F protein and monoclonal antibody purification. Monoclonal antibodies derived from mouse hybridomas were purified by protein A chromatography utilizing 0.1 M glycine, pH 2.8 as the eluant. Monoclonal antibodies derived from hamster hybridomas were purified by mercaptoethylpyridine (Ciphergen, Freemont, Calif.) chromatogrpahy utilizing 50 mM citrate, pH 4.0 as the eluant. Soluble F protein was purified from cell culture supernatants by affinity chromatography utilizing hamster monoclonal antibodies against hmpv F protein. Hamster monoclonal antibodies 121-1071-133 or 121-757-243 were attached to cyanogen bromide activated agarose at a density of 1-2 mg/ml of resin according to the manufacturer's instructions (Amersham, Piscataway, N.J.). Culture supernatants were applied to the resin and eluted with 0.1 M glycine, pH 2.8.

Serologic Assays. An ELISA assay was performed using hMPV-infected WI-38 cell monolayers. Cell monolayers were infected in 96 well plates at a multiplicity of infection of 1.0 and were incubated for 3-5 days post infection. The supernatants were removed, the cells were desiccated at 37° C., and stored at 4° C. until use. The plates were blocked using PBS containing 0.1% (v/v) Tween-20 and 0.5% (w/v) BSA. Diluted serum samples or hybridoma supernatants were incubated for 1 hour on the plates and washed with PBS/Tween. The plates were incubated with a peroxidase conjugated anti-mouse or anti-Armenian hamster biotinylated antibody (Jackson lmmunoReasearch, West Grove, Pa.) for an additional hour. For the hamster samples an additional incubation step with streptavidin-HRP (Amersham, Piscataway, N.J.) was added prior to color development with TMB substrate.

To determine the serum concentration of injected antibodies, capture ELISA assays were performed as follows: 100 μl of a 0.5 mg/ml solution of sF_{NL\1\00} in PBS buffer was coated onto Maxisorb microtiter plates overnight at 4° C. The following day the plates were blocked using 1% casein in PBS. Serum samples from in vivo challenge studies were diluted into PBS buffer and applied to the plate. A standard curve was generated using matched antibody in the same concentration range diluted into control normal hamster serum to calculate the serum concentration of antibody. Anitbody concentrations were caluculated using SoftMax pro software (Molecular Devices, Sunnyvale, Calif.).

Neutralization Assays. Serial 2 fold dilutions of serum, purified antibodies or hybridoma supernatants were incubated with 50-1000 TCID₅₀ of virus at 37° C. for 1 hour. After incubation, the virus-antibody mixtures were added to monolayers of Vero cells in 96 well plates, and the plates were centrifuged at 2000×g for 15 minutes at 25° C. The medium was removed from the cells, the cells washed in fresh medium without FBS, and finally overlaid with MEM medium without FBS supplemented with trypsin at 2.5 μg/ml. The cells were grown for 5-7 days at 37° C. after which the medium was removed and the cells were fixed by the addition of 80% acetone. Cells were fixed at 4° C. for 20 minutes and the plates were dried in air. The plates were blocked with 1% (w/v) casein (Pierce, Rockford, Ill.) and probed with a biotinylated monoclonal antibody against the hMPV fusion protein. A streptavidin-horseradish peroxidase conjugate was used for detection by TMB reagent. EC50 calculations were performed using Graphpad Prism software (GraphPad Software, San Diego, Calif.).

Biacore analysis—In a comparative study, the binding of individual 200 nM solutions of mAbs to immobilized sF_{NL\1\99} (2374 RUs immobilized) or sF_{NL\1\00}(2566 RUs immobilized) were characterized. In these experiments, a 250 μL injection of each antibody solution was passed over these surfaces, connected in series, at a flow-rate of 75 μL/min. Between injections, the F-protein surfaces were regenerated with a 1 min. pulse of 1M NaCl/50 mM NaOH. Kinetic analysis was also performed, to determine the rate and binding constants for the interactions of monoclonal antibodies 168-A5-338-284 and 168-A5-234-114 to immobilized sF_{NL\1\99} and sF_{NL\1\00} at an immobilization density between 80 and 300 RU. Flow rates, injection volumes and regeneration conditions were as above, and either 10 or 15 minutes of dissociation data was collected. Individual rate constants were calculated using the BIAevaluation 3.2 software (Biacore, Piscataway, N.J.).

In vivo assessment of protection. Golden Syrian hamsters, 6-8 wks old, were injected with 100 μl of purified monoclonal antibody or bovine serum albumin intramuscularly the day prior to challenge. The following day, the animals were anesthetized with isoflurane, bled and 100-200 μl of hMPV virus (10⁶-5×10⁷ TCID₅₀) was instilled intranasally. Four days post infection, the animals were euthanized by CO2 asphyxiation and the lungs were removed and homogenized. The level of hMPV in the lung homogenates was determined by TCID₅₀ analysis on LLC-MK2 cells. Serum levels of injected IgG were determined by capture ELISA (see above, under Serological Assays).

### REFERENCES CITED IN MATERIALS AND METHODS

1. van den Hoogen B G, Herfst S, Sprong L, Cane P A, Forleo-Neto E, de Swart R L, Osterhaus A D, Fouchier R A. (2004) Antigenic and genetic variability of human metapneumoviruses. Emerg Infect Dis 10,658-666.

2. Tang R S. Schickli J H. MacPhail M. Fernandes F. Bicha L. Spaete J. Fouchier R A. Osterhaus A D. Spaete R. Haller A A. (2003) Effects of human metapneumovirus and respiratory syncytial virus antigen insertion in two 3′ proximal genome positions of bovine/human parainfluenza virus type 3 on virus replication and immunogenicity. Journal of Virology. 77,10819-10828

3. de St Groth, F S and Scheidegger, D. Production of monoclonal antibodies: strategy and tactics Journal of Immunological Methods 35(1980), 1-21.

4. Bebbington C, et al. High level expression of a recombinant antibody from myeloma cells using a glutamine synthetase gene as an amplifiable selectable marker. Bio/Technology 1992; 10: 169-175.

5. Karber, G. (1931) 50% end-point calculation. Arch. Exp. Pathol. Pharmak., 162, 480-483

6.2. Results

Generation of HMPV F Protein Specific Antibodies

Different immunization techniques, i.e., DNA immunization, infection with a chimeric virus expressing the F protein, immunization with transfected cells expressing the F protein, infection with mammalian metapneumovirus, immunization with MPV-infected cells, immunization with Adenovirus-vectored MPV F protein, and immunization with hMPV F protein were employed. Immunizations that resulted in neutralizing monoclonal antibodies are listed in Table 3.

TABLE 3
Immunizations That Resulted In Neutralizing mAbs

| | | Microneutralization | | |
|---|---|---|---|---|
| | | of immune serum | | |
| | | NL\1\00 | NL\1\99 | |
| Animal | Immunizations | (A1) | (B1) | mAbs obtained |
| Hamster | NL\17\00 IN | 1:1250 | <1:50 | 121-1017-133 |
| | b/h PIV3/hMPV | | | 121-757-243 |
| | FNL\1\00 | | | 121-1025-257 |
| | IN/IP | | | |
| Hamster | NL\1\99 IN | 1:100 | 1:800 | 167-B3-836-141 |
| | NL\1\00 IN | | | 167-B3-659-315 |
| | Adenovirus | | | 167-B3-648-229 |
| | FNL\1\00 | | | 167-B3-967-127 |
| | and FNL\1\99 | | | |
| | sFNL\1\00 and | | | |
| | sFNL\1\99 | | | |
| Mouse | NL\1\99 IN | 1:100 | 1:200 | 168-A5-338-284 |
| | b/h PIV3/hMPV | | | 168-A5-234-114 |
| | FNL\1\00 IN | | | 168-A5-224-175 |
| | Adenovirus | | | |
| | FNL\1\00 | | | |
| | and FNL\1\99 | | | |
| | sFNL\1\00 and | | | |
| | sFNL\1\99 | | | |
| Mouse | NL\1\99 IN | 1:50 | 1:1600 | 168-B5P-710-202 |
| | NL\1\00 IN | | | 168-B5M-344-127 |
| | Adenovirus | | | 168-B5M-628-312 |
| | FNL\1\00 | | | |
| | and FNL\1\99 | | | |
| | sFNL\1\00 and | | | |
| | sFNL\1\99 | | | |

Neutralizing Effect of HMPV F Protein Specific Antibodies

The neutralization titers of different hMPV F protein specific antibodies against different hMPV subtypes, i.e., A1, A2, B1, and B2, was determined and are shown in Table 4 (data are represented in microgram per ml) and Table 5 (data are represented in nM).

TABLE 4
Neutralization Titers of hMPV F Protein specific antibodies

| | A1 | A2 | B1 | B2 |
|---|---|---|---|---|
| | NL\1\00 | NL\17\00 | NL\1\99 | NL\1\94 |
| mAb | IC₅₀ μg/ml | IC₅₀ μg/ml | IC₅₀ μg/ml | IC₅₀ μg/ml |
| 121-1017-133 | 16.65 | 38.40 | 12.75 | 29.55 |
| 121-757-243 | 11.85 | 10.35 | 1.35 | 5.10 |
| 121-1025-257 | 1.80 | 48.60 | >150.00 | ND |
| 167-B3-648-229 | 0.15 | 0.18 | >150.00 | 3.60 |
| 167-B3-659-315 | 16.95 | 1.95 | 2.40 | 19.05 |
| 167-B3-836-141 | 36.15 | 7.20 | 3.00 | 39.60 |
| 167-B3-967-127 | 0.15 | 0.15 | >150.00 | 4.80 |
| 168-A5-224-175 | 10.35 | 23.55 | 0.195 | 0.99 |
| 168-A5-234-114 | 0.585 | 1.155 | 0.0075 | 0.18 |
| 168-A5-338-284 | 0.375 | 0.81 | 0.03 | 0.165 |
| 168-B5M-344-127 | >150.00 | ND | 0.09 | 24.00 |
| 168-B5M-628-312 | 0.39 | 1.725 | 0.15 | 0.15 |
| 168-B5P-710-202 | 33.75 | 121.35 | 2.40 | 16.35 |

TABLE 5
Neutralization Titers of hMPV F Protein specific antibodies

| | A1 | A2 | B1 | B2 |
|---|---|---|---|---|
| | NL\1\00 | NL\17\00 | NL\1\99 | NL\1\94 |
| mAb | IC₅₀ nM | IC₅₀ nM | IC₅₀ nM | IC₅₀ nM |
| 121-1017-133 | 111 | 256 | 85 | 197 |
| 121-757-243 | 79 | 69 | 9 | 34 |
| 121-1025-257 | 12 | 324 | >1000 | nd |
| 168-A5-338-284^{(a)} | 2.5 | 5.4 | 0.2 | 1.1 |
| 168-A5-234-114^{(a)} | 3.9 | 7.7 | 0.05 | 1.2 |
| 168-A5-224-175^{(b)} | 69 | 157 | 1.3 | 6.6 |
| 168-B5P-710-202 | 225 | 809 | 16 | 109 |
| 167-B3-836-141 | 241 | 48 | 20 | 264 |
| 167-B3-311-311 | >1000 | nd | >1000 | >1000 |
| 168-B5M-344-127 | >1000 | nd | 0.6 | 160 |
| 168-B5M-628-312 | 2.6 | 11.5 | <1.0 | <1.0 |
| 167-B3-659-315 | 113 | 13 | 16 | 127 |
| 167-B3-648-229 | <1.0 | 1.2 | >1000 | 24 |
| mAb | A1 | A2 | B1 | B2 |
| | NL\1\00 | NL\17\00 | NL\1\99 | NL\1\94 |
| | IC₅₀ nM | IC₅₀ nM | IC₅₀ nM | IC₅₀ nM |
| 167-B3-967-127 | <1.0 | <1.0 | >1000 | 32 |

1 nM is 0.15 μg/ml
^{(a)}Only one of each clone is represented here.
^{(b)}Multiple clones of same sequence were isolated.

Thus, the hMPV F protein specific antibodies are highly neutralizing against A and B subtypes of hMPV.

Determination of Binding Affinities of HMPV F Protein Specific Antibodies

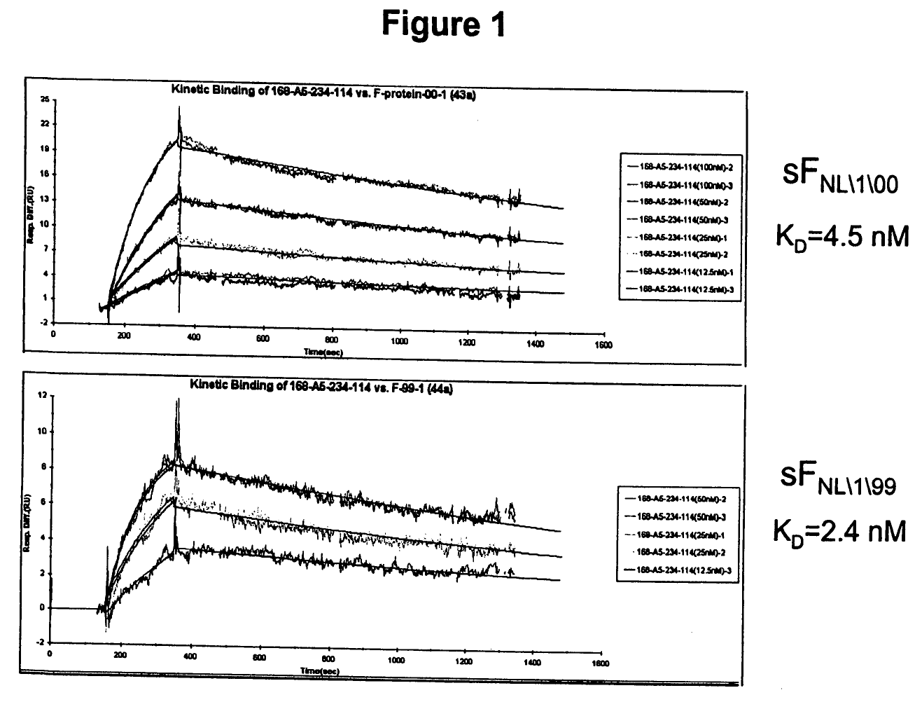

The kinetics of the binding affinity of hMPV F protein specific antibody 168-A5-234-114 (mAb234) to soluble F protein of the NL1/00 and NL1/99, respectively, were determined using Biacore analysis. The results are shown in FIG. 1.

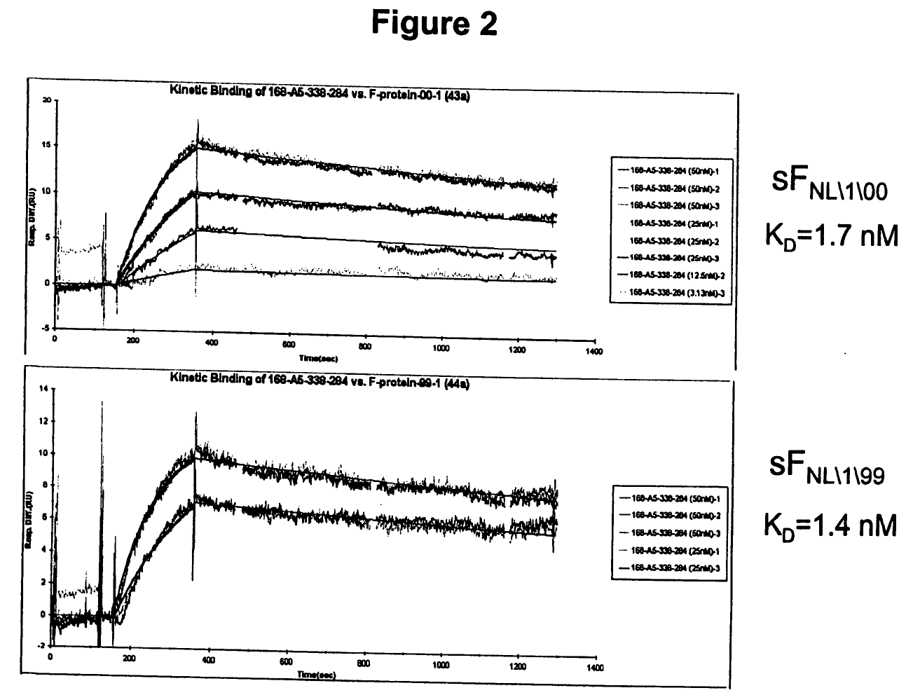

The kinetics of the binding affinity of hMPV F protein specific antibody 168-A5-338-284 (mAb338) to soluble F protein of the NL/1/00 and NL1/99, respectively, were determined using Biacore analysis. The results are shown in FIG. 2.

Thus, the hMPV F Protein specific antibodies bind specifically and with high affinity to the F protein of hMPV.

Table 6 shows the dissociation constants for binding between different hMPV F protein specific antibodies to hMPV F protein.

TABLE 6

| | | | | | | number |
|---|---|---|---|---|---|---|
| | | | | | | of curves |
| mAb | Surface | Immob. RUs | Ka(1/Ms) | Kb(1/s) | KD(M) | fitted |
| 168-A5-338-284 | F-99-1 | 300 | 1.34E+05 | 2.93E−04 | 2.18E−09 | 11 |
| 168-A5-338-284 | F-99-1 | 80 | 1.92E+05 | 2.72E−04 | 1.42E−09 | 5 |
| | | | average = 1.63E5 | average = 2.825E−4 | averageKD = 1.73E−9 | |
| 168-A5-338-284 | F-00-1 | 134 | 1.58E+05 | 2.76E−04 | 1.74E−09 | 8 |
| 168-A5-234-114 | F-99-1 | 146 | 7.89E+04 | 3.02E−04 | 3.83E−09 | 5 |
| 168-A5-234-114 | F-99-1 | 80 | 1.92E+05 | 4.63E−04 | 2.41E−09 | 5 |
| | | | average = 1.35e5 | average = 3.825E−4 | averageKD = 2.83E−9 | |
| 168-A5-234-114 | F-00-1 | 134 | 7.83E+04 | 3.52E−04 | 4.49E−09 | 8 |

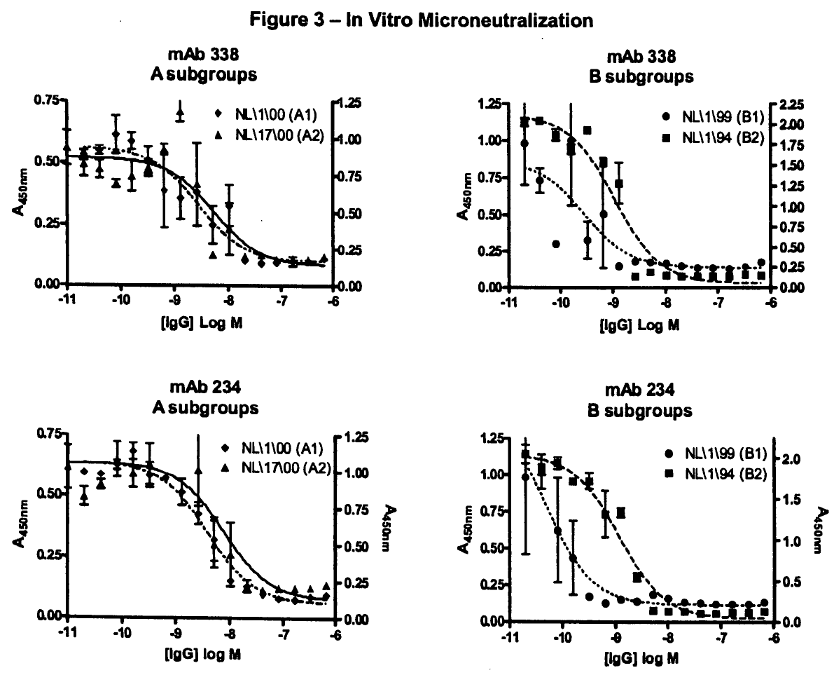

FIG. 3 shows a comparison of the microneutralization of A and B subtypes of virus. Antibodies were serially diluted 2 fold starting at 0.6 μM (100 μg/ml) and mixed with between 50-1000 TCID₅₀ of each individual virus. Neutralization of virus was determined by cell staining following a 5 day infection on Vero cells. Infection of cells was assessed by staining for hMPV fusion protein using biotinylated anti-hMPV fusion protein antibodies. Streptavidin-HRP was used to visualize F protein expression using the substrate TMB. IC₅₀ concentrations were determined using Graphpad Prism line fitting.

A comparison between the properties of Synagis®, NuMax™, mAb338, and mAb234 is shown in Table 7.

TABLE 7
Comparison of RSV and HMPV Neutralizing mAbs

| | Synagis ® | NuMax ™ | 168-A5-338 | 168-A5-234 |
|---|---|---|---|---|
| K_{D} | 1.4 nM | 0.02 nM | 1.7 nM A1 | 4.5 nM A1 |
| | | | 1.4 nM B1 | 2.4 nM B1 |
| IC₅₀ in vitro | 0.5 μg/ml | 0.03 μg/ml | 0.4 μg/ml A1 | 0.6 μg/ml A1 |
| neutralization | | | 0.03 μg/ml B1 | 0.01 μg/ml B1 |
| Protection in | 3 mg/kg | 1 mg/kg | ≦1 mg/kg | ≦1 mg/kg |
| animal model | 30 μg/ml | 10 μg/ml | 4 μg/ml serum | 5 μg/ml serum |
| | serum | serum | | |

In Vivo Protection Against NL\1\00 Challenge

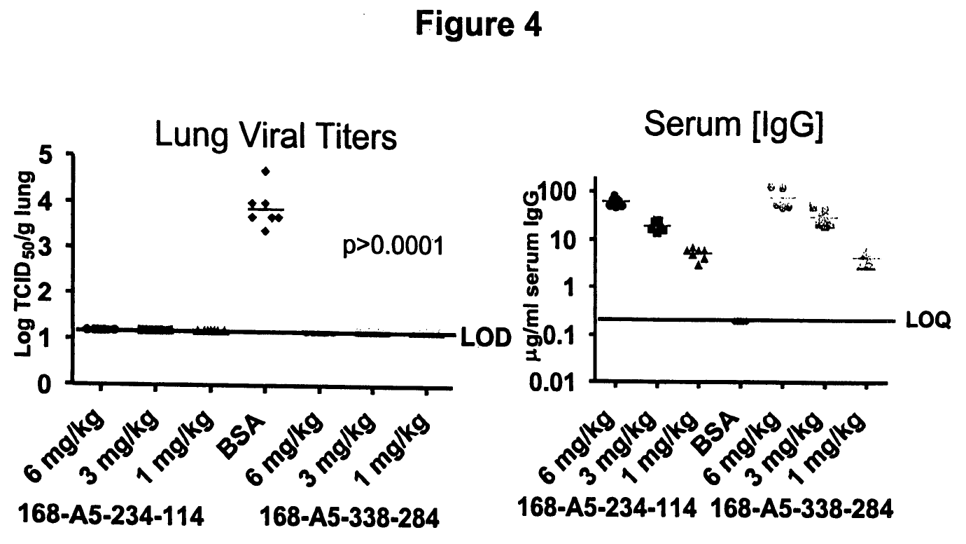

In vivo protection by hMPV F protein specific antibodies against hMPV challenge in hamsters was determined as described above. Lung viral titers and serum IgG are shown in FIG. 4. At Day 1 Golden Syrian hamsters were injected intramuscularly with varying doses of mAb 338, mAb 234 or BSA. The following day the hamsters were challenged with 1×10⁷ TCID₅₀ of NL\1\00 strain of hMPV intranasally. Four days post-infection the lungs were removed, homogenized and subjected to TCID₅₀ titration of the virus. Serum samples for measurement of IgG concentrations were taken prior to challenge. IgG concentrations were obtained using a sandwich ELISA method with the capture antigen as sF_{NL\1\00} and the detection reagent as anti-mouse IgG-HRP conjugate. IgG concentrations were determined relative to a standard curve of the corresponding mAb spiked into normal hamster serum. (See FIG. 4).

Thus, the hMPV F protein specific antibodies protect against hMPV infection in animal model systems.

Identification and Sequencing of Monoclonal Antibody-Resistant Mutants

Monoclonal antibody resistant mutants (MARMs) were isolated from incubation with the mAb338. Sequencing of the mutants revealed the following mutations as conferring resistance (i) to mAb338: A238E and K242N, A238E and K242T, A238T and K242T, K242N or I241R; and (ii) to mAb234: K242N. A sequence alignment of the native HMPV F proteins is shown in FIG. 5 (SEQ ID NOs:127-131). FIG. 5 also shows an alignment between the hMPV F proteins and the RSV F protein. The amino acid positions of mutations that confer resistance to different antibodies are also indicated by underlining. These mutations are located in analogous positions of the hMPV and the RSV F proteins. Thus, the antigenic structure of the F protein is conserved between RSV and hMPV.

a. Marm data is shown below (mutated residues are shown by underlining):

| | |
|---|---|
| NL\1\00 WT | P T S A G Q I K L M |
| MARM 4 | P T S A G Q R K L M |
| MARM 47 | P T S A G Q I N L M |
| MARM 64 | P T S E G Q I N L M |
| MARN 69 | P T S T G Q I N L M |
| MARM 76 | P T S A G Q R K L M |
| MARM 86 | P T S E G Q I T L M |

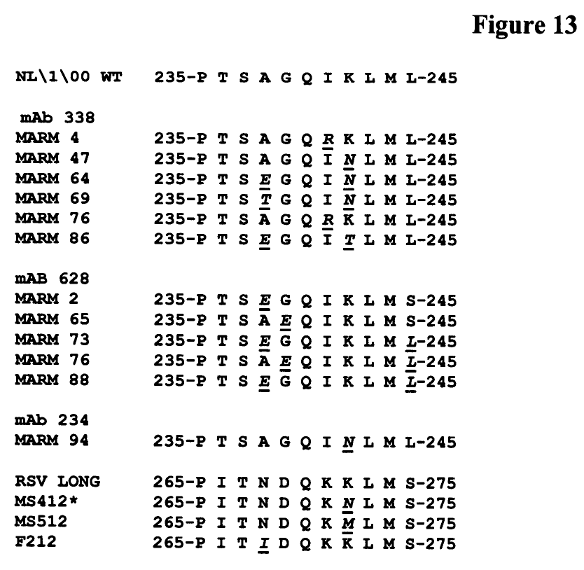

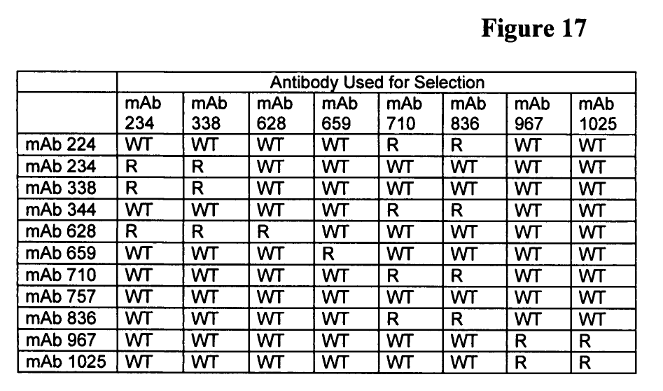

Additional MARM data are shown in FIGS. 13 to 16. As shown in FIG. 17, mutations that confer resistancy against the antibody that was used to select the MARM can also confer resistancy to other antibodies.

7. Isolation and Characterization of Monoclonal Antibodies which Neutralize Human Metapneumovirus in Vitro and in Vivo

7.1. Introduction

Human metapneumovirus (hMPV) is a recently described member of the Paramyxoviridae family. This virus is in the pneumovirinae subfamily with respiratory syncytial virus (RSV) and shares many common features with RSV. hMPV causes respiratory tract illness that occurs predominantly in the winter months with-symptoms that range from mild to severe cough, bronchiolitis and pneumonia. Based on sequence data isolates of hMPV and RSV segregate into 2 subgroups, A and B, and a further bifurcation exists within the A and B groupings for hMPV. Due to the high level of sequence conservation across all the viral subgroups, the fusion (F) protein is likely to be the dominant antigenic determinant that can be targeted to generate cross-subgroup neutralizing antibodies. It has been shown that a single monoclonal antibody directed at the RSV F protein can prevent severe lower respiratory tract infection by RSV in both animals and humans. A panel of neutralizing monoclonal antibodies against the F protein of hMPV has been generated; the antibodies can inhibit viral replication in vitro and a subset of them can protect against challenge with both A and B subtypes of hMPV in vivo. These antibodies could be divided into six distinct groupings based on results of competitive binding experiments with hMPV-infected cells. These neutralizing antibodies were used to generate hMPV escape mutants in order to map the neutralization epitopes on the F protein. Many of these epitopes were localized to regions on the hMPV F protein homologous to previously defined neutralizing epitopes on the F protein of RSV. These data suggest a conservation in the antigenic structure of the F protein among different members of the pneumovirinae subfamily.

7.2 Materials and Methods:

Cells and Virus. Vero, WI-38, LLC-MK2 cells that were used for the propagation of hMPV and PIV3-derived viruses were maintained in Eagle modified minimal essential medium (EMEM) supplemented with 10% fetal bovine serum (FBS). Adenovirus vectors were grown in HEK-293 cells grown in Dulbecco's modified Eagle medium (DMEM)+10% FBS. The mouse myeloma cell line, NSO, was maintained in DMEM+20% FBS; myeloma fusion cell lines were maintained in Excell 610 (JRH Biosciences, Lenexa, Kans.)+10% FBS. Titers of viral stocks were determined by TCID₅₀ measurement (12) on Vero cells. Viral infection was determined by reactivity with antibodies directed against hMPV as described in subsequent sections.

For the propagation hMPV, semiconfluent cell monolayers were infected at a multiplicity of infection of 0.1 TCID₅₀/cell in EMEM plus 2.5 μg/ml trypsin without FBS; 5-9 days post-infection the virions were harvested by freeze-thaw disruption of the cells. Viral samples were stabilized by the addition of 10× SPG (2.18 M sucrose, 0.038 M KH₂PO₄, 0.054 M L-glutamate) and stored at −80° C. The prototype hMPV strains (provided by A. Osterhaus) studied were: A1 NL\1\00, A2 NL\17\00, B1 NL\1\99 and B2 NL\1\94.

Parainfluenza virus 3 (PIV3)-vectored hMPV F protein virus (b/hPIV3/hMPV F) has been reported previously and was propagated as described in Vero cells (21). Estimation of the viral concentration of PIV3 constructs was estimated by determining plaque forming units per milliliter of viral stock on Vero cells. Adenovirus constructs expressing the F protein from strain NL\1\00 and NL\1\99 sequences were produced using the AdEasy adenoviral system with the transfer vector pShuttle-CMV (AdEasy, Stratagene, LaJolla, Calif.). The resultant adenovirus was propagated in HEK-293 cells according to the manufacturer's instructions. Viral titers for adenovirus were determined using a TCID₅₀ assay with cytopathic effect (CPE) as the readout.

Production of Hybridoma Cell Lines. Armenian hamsters (Cytogen Research and Development, Inc. Boston, Mass.) and BALB/c mice (Jackson Laboratory, Bar Harbor, Me.) were immunized using a combination of some or all of the following: intranasal infection with hMPV at 10⁶ TCID₅₀ per animal of either NL\1\7\00, NL\1\00 or NL\1\99, intranasal infect with 10⁶ PFU b/hPIV2/hMPV F_{NL\1\00}, intraperitoneal injection with adenovirus-vectored hMPV F_{NL\1\00} or hMPV F_{NL\1\99} at a dose of 9×10⁷ TCID₅₀, purified soluble hMPV F protein derived from NL\1\00 and NL\1\99 sequences injected intraperitoneally with either GERBU MM adjuvant (CC Biotech, Valley Center, Calif.) or in an adjuvant-free solution. Four days after the final immunization, splenic lymphocytes were isolated and fused to NS0 cells using polyethylene glycol as described previously (6). Fusions were plated either in semi solid medium (ClonaCell, Stem Cell Technologies, Vancouver, BC) or in liquid medium in 96 well plates. Hybridoma supernatants that produced hMPV-specific antibodies were identified by ELISA on hMPV-infected cells.

Identification and Sequencing of monoclonal antibodies (mAb)—RNA was isolated from hybridoma cells expressing the antibodies of interest using the RNAeasy system (Qiagen, Germantown, Md.). The CDR sequences were amplified by PCR using commercially available probes (EMD Biosciences, La Jolla, Calif.) and were cloned into topoisomerase-bound TA overhang plasmid vectors (Invitrogen, Carlsbad, Calif.). Multiple clones of the CDR containing plasmid vectors were isolated and sequenced using BigDye Terminator v3 (ABI, Foster City, Calif.) reactions and run on either an ABI 3100 or ABI 3730 sequencer to derive a consensus sequence of the hypervariable regions.

mAb purification—Hamster monoclonals were purified on MEP Hypercel (Pall Corp., East Hills, N.Y.) columns using 50 mM citrate at pH 4.0 to eluate the mAb; eluates were immediately neutralized with 1:10 volume of 1 M Tris-HCl, pH 8.0. Mouse monoclonals were purified on protein A sepharose; mouse IgG₁ were loaded in hybridoma medium containing 50 mM Tris, pH 8.5 and 1 M NaSO₄ whilst all other mouse subtypes were loaded directly from hybridoma medium. The protein A columns were eluted with 0.1 M glycine, pH 2.8 and the eluates were neutralized immediately with 1:10 volume of 1 M Tris-HCl, pH 8.0.

HMPV F protein construct generation. Full length and truncates of F protein that lacked the transmembrane domain were made using plasmids RF516 and RF515 containing full length sequences of the fusion protein from isolates NL\1\00 and NL\1\99 respectively (from the laboratory of A. Osterhaus) as the template for PCR reactions. To obtain a soluble histidine-tagged form of the hMPV F protein, the following oligonucleotides were used to generate clones; from the NL\1\00 sequence, 5′ AACCAAAAGCTTCACCATGTCTTGGAAAGTGGTGATC 3′ and 5′ TTAATTGAATTC TTAGTGATGGTGATGGTGATGGCCAGTGTTTCCTTTCTCTGC 3′ and from the NL\1\99 sequence, 5′TTCCTTAAGCTTCACCATGTCTTGGAAAGTGATGATCATC 3′ and 5′TTAATTGGATCCTTAGTGATGGTGATGGTGATGACCAGTGTTTCCTTTTTCTGCA CT 3′. The PCR products were cleaved using the restriction endonucleases EcoRI and HindIII for the NL\1\00 sequence, and BamHI and HindlIl for the NL\1\99 sequence, and then ligated to the vector pcDNA3.1(+) cleaved with the same endonucleases. HEK-293 cells were transiently transfected with the pcDNA clones, using lipofectamine 2000 (Invitrogen, Carlsbad, Calif.) to introduce the DNA into the cells. To make stable hMPV F protein-expressing constructs, the same plasmid source of DNA was used to generate PCR products, however, alternate primers were used for both the NL/1/00 and NL/1/99 sequences; 5′-AATCAACGGTCCGCCACCATGTCTTGGAAAGTG-3′ and 5′ TTAATTGAATTCTTAGTGATGGTGATGGTGATGGCCAGTGTTTCCTTTCTCTGC 3′. The PCR products were cleaved with RsrII and EcoRI and ligated to the pEE15.1 (Lonza, Allendale, N.J.) vector cleaved with the same restriction endonucleases. Stable NS0 cell lines were made as describe by Bebblington (2). Full-length F protein constructs were made using the following oligonucleotides: For NL\1\00 5′ AACCAAAAGCTTCACCATGTCTTGGAAAGTGGTGATC 3′ and 5′ AATTAAGGATCCTAATTATGTGGTATGAAGCCATT 3′ and for NL\1\99 5′TTCCTTAAGCTTCACCATGTCTTGGAAAGTGATGATCATC 3′ and 5′ AATTAAGGATCCTAATTATGTGGTATGAAACCGCC. PCR products were cleaved with BamHI and HindIII endonucleases and were ligated to pcDNA3.1(+) cleaved with the same endonucleases. These vectors were used as the source of DNA for the construction of the adenovirus transfer vector pShuttle-CMV. The full length F protein containing fragments were obtained by cleavage of the pcDNA3.1 clones with the restriction endonucleases HindIII and EcoRV and were ligated to the pShuttle-CMV vector cleaved with the same endonucleases.

HMPV F protein purification—Histidine-tagged soluble F protein was initially purified by Ni—NTA (Qiagen, Germantown, Md.) chromatography which yielded protein that was 60% pure as determined by SDS-PAGE. Subsequently, after isolation of the F protein-specific monoclonal antibody (mAb1017), the F protein was purified by affinity chromatography on mAb 1017 coupled to cyanogen bromide-activated Sepharose and eluted with 0.1 M glycine, pH 2.8; the eluate was neutralized with 1:10 volume of 1 M Tris-HCl, pH 8.0 and was dialyzed into PBS. Affinity-purified F protein was >90% pure as judged by SDS-PAGE.

ELISA Assays An ELISA was developed to detect anti-hMPV antibodies in hybridoma supernatants or animal sera using hMPV-infected WI-38 cell monolayers. Cell monolayers in 96 well plates were infected with hMPV at a multiplicity of infection of 1.0 and were incubated subsequently for 3-5 days post infection. The supernatants were removed and the cells were desiccated at 37° C., and stored at 4° C. until use. For ELISA, the plates were blocked with PBS containing 0.1% (v/v) Tween-20 and 0.5% (w/v) BSA. This and all subsequent steps were performed at room temperature. Diluted serum samples or hybridoma supernatants were incubated on the plates for 1 hour and the plates were then washed with PBS/Tween. Horseradish peroxidase (HRP) conjugated anti-mouse or anti-Armenian hamster biotinylated antibody (Jackson lmmunoReasearch, West Grove, Pa.) was added, the plates were incubated for an additional hour and then washed. For the hamster samples streptavidin-HRP (Amersham Biosciences, Piscataway, N.J.) was added and incubated for 1 hour. Plates were developed with Sure Blue TMB substrate (KPL, Gaithersburg, Md.). End point titers of serum samples were defined as the last dilution that achieved a minimal 2-fold absorbance over the control absorbance.

Competition ELISA experiments were performed using biotinylated mAb 242, mAb 338, Mab 659, mAb 757, mAb 836, mAb 1017 and mAb 1025. Antibodies were biotinylated using either biotin-XX-SSE (Invitrogen, Carlsbad, Calif.) or Biotin-XX-SE (Vector Laboratories, Burlingame, Calif.) according to the manufacturers' instructions. A standard binding curve for each of the biotinylated antibodies generated on hMPV infected WI-38 cells using strapavidin-HRP as the detection reagent. The concentration of the biotinylated antibodies used in the competition experiment gave a half maximal signal on the standard curve. In the competition ELISAs, the competing monoclonals were used at concentrations ranging from 50 to 0.03 μg/ml. Unlabeled competitive mAb that gave a >50% reduction in signal at a concentration less than or equal to 100 times the biotinylated antibody concentration were rated as competing.

To determine the serum concentration of injected antibodies, capture ELISA assays were performed as follows: hMPV soluble F protein (50 ng/well) from NL\1\00 was coated onto Nunc Maxisorb (Nalge Nunc, Rochester, N.Y.) microtiter plates overnight at 4° C. in PBS buffer (Pierce, Rockford, Ill.). The following day the plates were blocked using 1% casein in PBS. Serum samples were diluted into PBS and applied to the plate. A standard curve generated using matched antibody in the same concentration of normal hamster serum was used to calculate the serum concentration of antibody. Anti-mouse HRP conjugate was used for detection with SureBlue TMB reagent.

Neutralization Assays. Serial 2 fold dilutions of serum, hybridoma supernatants, or purified antibodies were incubated with 50-1000 TCID₅₀ of virus at 37° C. for 1 hour. After incubation, the virus-antibody mixtures were added to monolayers of Vero cells in 96 well plates; the plates were then centrifuged at 2000×g for 15 minutes at 25° C. The medium was removed from the cells, the cells were washed in fresh medium without FBS and finally overlaid with EMEM medium without FBS and supplemented with trypsin at 2.5 μg/ml. The cells were grown for 5-7 days at 37° C., after which the medium was removed and the cells were then fixed by the addition of 80% acetone at 4° C. for 20 minutes; after this the plates were air-dried. Prior to development the plates were blocked with 1% (w/v) casein and then probed with either a polyclonal sera obtained animals immunized with virus or biotinylated mAb1017. A streptavidin-horseradish peroxidase conjugate was used to detect the biotinylated antibody. Alternatively, an anti-species specific secondary antibody conjugated to horseradish peroxidase was used for detection with polyclonal sera. The plates were developed using by Sure Blue reagent. For hybridoma supernatants or polyclonal sera the neutralization titer is defined as the last dilution that gives an absorbance that is less than 2 fold over the uninfected control cell absorbance. IC₅₀ values were determined using GraphPad Prism software using curve-fitting for a non-linear sigmoid dose response.

Biacore analysis—Kinetic analysis was performed to determine the binding constants for antibodies mAb338 and mAb234 to immobilized soluble hMPV F protein. Soluble F_{NL\1\00} and soluble F_{NL\1\99} proteins were immobilized on CM5 sensor chips (BiaCore, Uppsala Sweden) using an amine coupling kit as described previously (11) at an immobilization density between 80 and 300 RU. Excess reactive esters were quenched with 70 μl of a 1 M ethanolamine hydrochloride, pH 8.5 solution. The surfaces were connected to a BiaCore 3000 in series. 250 μL of each mAb solution were injected at concentrations ranging from either 0.39 nM to 400 nM, or from 3.13 nM to 100 nM were at a flow-rate of 75 μL/min, and 15 minutes of dissociation data was collected. Between injections, the surfaces were regenerated with a 1 minute pulse of 1M NaCl-50 mM NaOH. Data was analyzed using the BIAevaluation software, supplied by BIAcore, Inc.

In vivo assessment of protection. 6-8 week old Golden Syrian hamsters (6-7 animals/group) were injected intramuscularly with various concentrations of purified monoclonal antibody or bovine serum albumin in a volume of 100 μl the day prior to challenge. The following day the animals were anesthetized with isoflurane, bled, and 100-200 μl of virus (1×10⁷ TCID₅₀/ml) was instilled intranasally. Four days post infection the animals were euthanized by CO₂ asphyxiation and the lungs removed and homogenized in Hank's balanced salt solution using a dounce homogenizer. Nasal turbinates were isolated and ground using a mortal and pestle in Hank's balanced salt solution. TCID₅₀ determinations from lung and nasal turbinate homogenates were performed as follows: homogenates and sequential 10 fold dilutions of the homogenates were applied to washed LLC-MK2 cells and incubated for 1 hour at room temperature. The supernatants were removed and cells overlaid with Opti-MEM (Invitrogen, Carlsbad, Calif.) medium containing 5 μg/ml of porcine derived trypsin (Biowhittaker, Walkersville, Md.). The cells were incubated at 37° C. for 6-7 days. The medium was removed and the cells fixed using 80% methanol. Plates were blocked in 5% non-fat dried milk for 30 minutes. Polyclonal sera raised from animals infected with hMPV were used for the staining of the cells. Species specific secondary antibody conjugated to HRP was used for detection using the 4CN peroxidase substrate (KPL, Gaithersburg, Md.). Infection was assessed by visual inspection of individual wells and presented as log₁₀TCID₅₀/gram tissue as previously described (19).

7.3 Results

A panel of monoclonal antibodies that was both specific for hMPV F protein, and which at low concentrations neutralized hMPV, was generated. Numerous immunization strategies were employed to elicit robust F protein-specific immune responses in both Armenian hamsters and BALB/c mice. In all cases, the first immunization was an intranasal infection with a wild type hMPV virus to prime the animals; this was followed by immunizations employing either recombinant adenovirus or recombinant bovine parainfluenza virus expressing hMPV F protein. In some cases, subsequent immunization with soluble recombinant hMPV F protein was carried out (Table 8). In this manner, high titer anti-F protein-specific responses in the animals could be generated. In addition, to generate antibody responses that were reactive to both subtypes of hMPV, immunizations containing both prototype A (NL\1\00) and prototype B (NL\1\99) F protein sequences were employed. As shown in Table 8, this immunization strategy resulted in the production of high-titered antibodies in both mice and hamsters that neutralized one or both types of hMPV.

TABLE 8
Immunizations producing serum titers and monoclonal antibodies.

| | | Endpoint Infected cell | | Microneutralization | | |
|---|---|---|---|---|---|---|
| | | ELISA titer² | | Titer² | | |
| | Immunizations¹ | NL\1\00 | NL\1\99 | NL\1\00 | NL\1\99 | mAbs |
| Animal | route immunogen | (A1) | (B1) | (A1) | (B1) | obtained |
| Hamster | IN NL\17\00 | 1:781,250 | 1:781,250 | 1:1250 | <1:50 | mAb 1017 |
| | IN/IP PIV3/F_{NL\1\00} | | | | | mAb 757 |
| | | | | | | mAb 1025 |
| Hamster | IN NL\1\99 | 1:25,600 | 1:25,600 | 1:100 | 1:800 | mAb 836 |
| | IN NL\1\00 | | | | | mAb 659 |
| | IP Adeno F_{NL\1\00}/F_{NL\1\99} | | | | | mAb 967 |
| | IP soluble F_{NL\1\00}/F_{NL\1\99} | | | | | |
| Mouse | IN NL\1\99 | 1:312,500 | 1:312,500 | 1:100 | 1:200 | mAb 338 |
| | IN PIV3/_{NL\1\00} | | | | | mAb 234 |
| | IP Adeno F_{NL\1\00}/F_{NL\1\99} | | | | | mAb 224 |
| | IP soluble F_{NL\1\00}/F_{NL\1\99} | | | | | |
| Mouse | IN NL\1\99 | 1:312,500 | 1:312,500 | 1:50 | 1:1600 | mAb 710 |
| | IN NL\1\00 | | | | | mAb 344 |
| | IP Adeno F_{NL\1\00}/F_{NL\1\99} | | | | | mAb 628 |
| | IP soluble F_{NL\1\00}/F_{NL\1\99} | | | | | |

¹Immunization routes were IN-intranasal and IP-intraperitoneal
²End point titers are determined as described in materials and methods.

Following the immunizations, the spleens of the mice and hamsters were fused to generate hybridoma cells and the hybridoma supernatants were screened for reactivity towards cells infected with the hMPV (NL\1\00) or uninfected cells. A minimal 5-fold differential in absorbance between infected and uninfected cells was used as the criterion to select antibodies for the next stage of analysis. Hybridoma supernatants which were reactive with infected cells were expanded and tested as unfractionated supernatants in viral neutralization assays. The hybridoma supernatants varied greatly in the quantity of antibody they contained, but were tested for hMPV neutralization without concentration adjustment. Therefore, this screening method selected for hybridomas that either produced high levels of antibody, or produced antibody at low concentrations but with high neutralization activity. Hybridomas supernatants that contained neutralization activity at greater than a 1:2 dilution against at least one hMPV type were cloned by limited dilution and were then expanded to generate antibody for purification and further analysis. Table 9 shows the IC₅₀ titers of all of the antibodies that could be isolated by limited dilution and that produced sufficient antibody to assess their potency. In four cases, the same monoclonal antibodies were isolated from multiple cell lines, as determined by rtPCR and sequencing of the heavy and light chains. A single isolate of each sequence was carried forward for full evaluation of neutralization potency. A wide range of neutralization potencies was seen and many of the isolated antibodies did neutralize one or more of the four prototype viruses tested. For the top three antibodies, mAb 338, mAb 234 and mAb 628, the neutralization capacity against all 4 prototype stains was lower than 2 μg/ml IC₅₀ demonstrating a strong pan neutralizing capacity. Another pan-neutralizing mAb, 1017, showed comparable neutralization across all 4 prototypes but was 25-100 fold less potent that the top 3 antibodies described above. Some antibodies show enhanced neutralization (10-100 fold lower IC₅₀) against B type viruses as seen for mAb 242, mAb 757, mAb 710 and mAb 344. mAb 1025 and mAb 967 are more potent at neutralizing A type viruses, however, they differ in the following ways: The mAb 1025 has essentially no neutralizing capacity against both B1 and B2 prototypes whereas the mAb 967 has the capacity to neutralize the B2 prototype but not the B1 prototype. Two antibodies, mAb 659 and mAb 836, show a difference in neutralizing capacity that is not split along the A and B subtypes. These antibodies neutralize the A2 and B1 subgroups better than the A1 and B2 subgroups suggesting that amino acid changes that are not subgroup specific are playing a role in the binding of these antibodies to their epitope.

TABLE 9
IC₅₀ determination of purified monoclonals¹

IC₅₀ μg/ml

| | A1 | A2 | B1 | B2 |
|---|---|---|---|---|
| mAb | NL\1\00 | NL\17\00 | NL\1\99 | NL\1\94 |
| mAb 338 | 0.38 | 0.8 | 0.03 | 0.17 |
| mAb 234 | 0.59 | 1.2 | 0.01 | 0.18 |
| mAb 628 | 0.39 | 1.7 | 0.15 | 0.15 |
| mAb 242 | 10.4 | 23.6 | 0.2 | 1.0 |
| mAb 757 | 11.9 | 10.4 | 1.4 | 5.1 |
| mAb 659 | 17.0 | 2.0 | 2.4 | 19.0 |
| mAb 836 | 36.2 | 7.20 | 3.0 | 39.6 |
| mAb 1017 | 16.7 | 38.4 | 12.8 | 29.6 |
| mAb 967 | 0.15 | 0.15 | >100 | 4.8 |
| mAb 710 | 33.8 | >100 | 2.4 | 16.4 |
| mAb 344 | >100 | >100 | 0.8 | 24.0 |
| mAb 1025 | 1.80 | 48.6 | >100 | >100 |

¹IC₅₀ determinations calculated as described in material and methods.

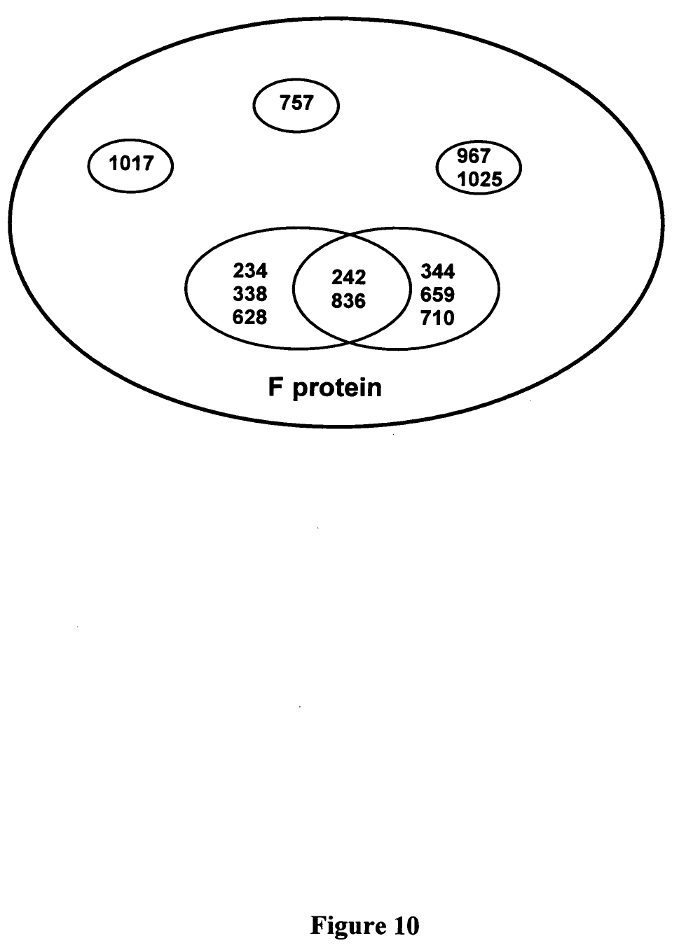

In order to determine the number of antigenic sites that these antibodies recognize, competition ELISA matrices were set up. Seven of the twelve isolates were biotinylated and the ability of unlabelled monoclonals to compete for binding to hMPV infected cells. The results of these experiments are shown in Table 10. In all there are 6 distinct patterns of competition. Two of the antibodies (mAb 1017 and mAb 757) are only competed by cold matched antibody. MAb 967 and mAb 1025 compete for the same or overlapping sites. The remainder of the monoclonals have a more complex order of competition. MAb 338 is competed by itself, mAb 234 and mAb 628, however, other biotinylated antibodies, mAb 242 and mAb 836, are competed by unlabelled mAb 338. MAb 242 and mAb 836 show the same pattern of competition but only differ in their pattern from mAb 659 in the ability to be competed by mAb338 and mAb628. This data support the epitope map shown in FIG. 10. This map illustrate the overlapping nature of the epitopes.

TABLE 10
Competition ELISA using biotinylated mAb¹

| | Biotinylated mAb | | | | | | |
|---|---|---|---|---|---|---|---|
| | mAb | mAb | mAb | mAb | mAb | mAb | mAb |
| | 242 | 338 | 659 | 757 | 836 | 1017 | 1025 |
| mab 234 | − | + | − | − | − | − | − |
| mAb 242 | + | − | + | − | + | − | − |
| mAb 338 | + | + | − | − | + | − | − |
| mAb 344 | + | − | + | − | + | − | − |
| mAb 628 | + | + | − | − | + | − | − |
| mAb 659 | − | − | + | − | − | − | − |
| mAb 710 | + | − | + | − | + | − | − |
| mAb 757 | − | − | − | + | − | − | − |
| mAb 836 | + | − | + | − | + | − | − |
| mAb 967 | − | − | − | − | − | − | + |
| mAb 1017 | − | − | − | − | − | + | − |
| mAb 1025 | − | − | − | − | − | − | + |

¹+ signs indicate competition as described in materials an methods.

Of all the hMPV F protein-specific monoclonal antibodies that were selected, antibodies 234 and 338 had the most potent virus neutralizing activity across all four hMPV subtypes (Table 9). To determine their binding characteristics, a Biacore analysis using immobilized soluble hMPV NL\1\00 or NL\1\99 F proteins (sF_{NL\1\00} and sF_{NL\1\99} respectively) was performed. The Kₒₙ, K_{off} and K_{d} values derived from the Biacore analysis are shown in Table 11; antibodies 234 and 338 showed comparable on and off rates, and nanomolar affinities for the F protein of both hMPV types.

TABLE 11
Biacore determinations of binding constants¹

| | | Immobilized | Kₒₙ | K_{off} | K_{D} |
|---|---|---|---|---|---|
| Antibody | Antigen | RU | (1/Ms) | (1/s) | (M) |
| mAb 234 | sF_{NL\1\99} | 80 | 1.92 × | 4.63 × | 2.41 × |
| | | | 10⁵ | 10⁻⁴ | 10⁻⁹ |
| mAb 234 | sF_{NL\1\00} | 134 | 7.83 × | 3.52 × | 4.49 × |
| | | | 10⁵ | 10⁻⁴ | 10⁻⁹ |
| mAb 338 | sF_{NL\1\99} | 80 | 1.92 × | 2.72 × | 1.42 × |
| | | | 10⁵ | 10⁻⁴ | 10⁻⁹ |
| mAb 338 | sF_{NL\1\00} | 134 | 1.58 × | 2.76 × | 1.74 × |
| | | | 10⁵ | 10⁻⁴ | 10⁻⁹ |

¹Binding constrants were determined and calculated as described in materials and methods using the Biaevaluation software.

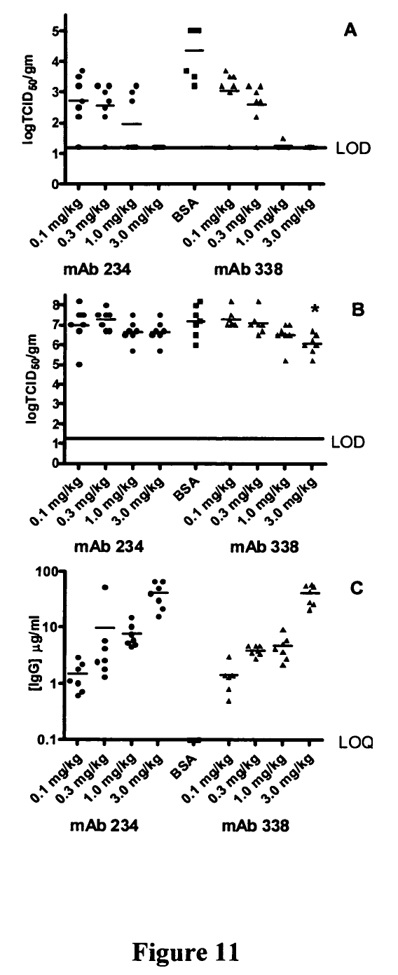

To examine further the ability of the antibodies to neutralize hMPV, mAbs 234 and 338 were tested in vivo in a prophylactic viral infection model using Golden Syrian hamsters as described previously (15). The mAbs were administered to Syrian hamsters (7 animals/group) by intramuscular injection, 24 hours prior to intranasal challenge with hMPV NL\1\00 at a dose of 1-2×10⁶ TCID₅₀. Control animals received BSA instead of antibody. Animals were euthanized four days post-challenge and the quantities of hMPV in the lungs and nasal turbinates of the animals were measured. Animals that had received either mAb 234 and 338 at doses greater than or equal to 3 mg/kg showed no detectable levels of virus in their lungs. To determine the minimum effective dose of the antibodies a dose titration of the antibody between 0.1 and 3.0 mg/kg is shown in FIG. 11. At 3.0 mg/kg, both mAbs 338 and 234 gave rise to a minimum three log reduction (relative to the BSA control) in lung virus titer (FIG. 11, panel A). At 1 mg/kg, mAb 338 still caused a minimum 3 log reduction in lung virus titer whereas mAb 234 caused an average 2 log reduction. Doses of 0.3 and 0.1 mg/kg resulted in higher levels of virus in the lungs; however, for these dose groups the reductions in lung virus titers relative to the control were statistically significant. Thus, antibodies 338 and 234 were able to decrease the viral burden in the lungs of animals at doses as low as 0.1 mg/kg. However, prevention of viral replication in the upper airways were much less marked and only seen with higher doses of antibody. The log₁₀TCID₅₀/gram of hMPV recovered from nasal turbinates of animals receiving intramuscular injection with mAbs 338 and 234 at doses between 3 and 0.1 mg/kg is shown in FIG. 11, panel B. A reduction in viral titers relative to the controls were observed at doses of either antibody of 3.0 and 1.0 mg/kg, but were statistically significant only for the 3 mg/kg dose of mAb 338.

The serum concentrations of the antibodies were determined 24 hr following intramuscular administration (FIG. 11 panel C). Serum samples were collected just prior to intranasal challenge with hMPV. As expected, higher doses of administered antibody resulted in higher concentrations of antibody measured in the serum of the animals. These data suggest that a serum concentration of mAb 338 between 5 and 10 μg/ml correlates with a minimum 3 log reduction in viral titer in the lungs of infected animals. mAb 234 appears to be slightly less potent at reducing virus in both the upper and lower airways, and a circulating concentration >10 μg/ml of this antibody is required to decrease the lung viral titers to undetectable levels.

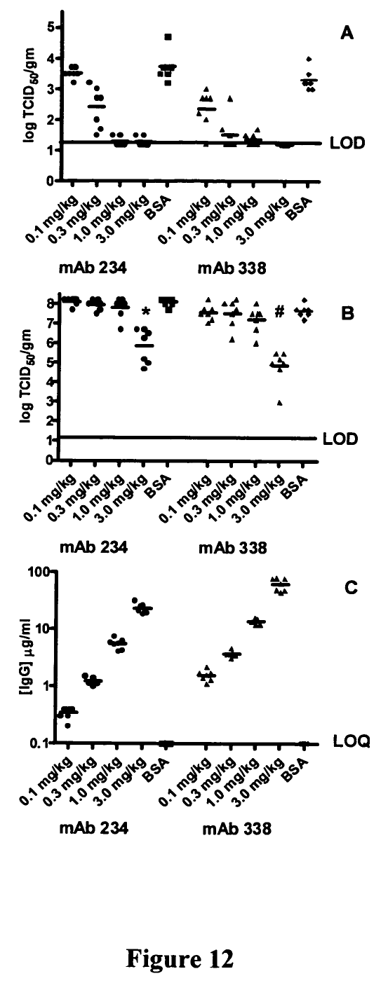

A similar experiment was performed using the B1 subgroup prototype virus. Syrian hamsters received an intramuscular injection with mAb 338 or mAb 234 24 hours prior to nasal challenge with NL\1\99. As in the previous experiment control animals received BSA, however, since the animals were not administered the antibodies on the same day a separate BSA control was performed for each antibody. The maximum level of hMPV NL\1\99 recoved from the lungs of the control animals was lower than that obtained with NL\1\00. Because of this, a 3 log reduction with either mAb 234 or mAb 338 due to the limit of detection of the assay could not be observed. Nevertheless, similar to what was seen with using the NL\1\00 virus, at doses of 3 mg/kg and 1 mg/kg both antibodies reduced the lung viral titers by greater than 2 logs (FIG. 12, panel A). Again, at a dose of 0.3 mg/kg there was a statistically significant reduction in lung viral titer with both the mAb 338 and mAb 234, however, at the 0.1 mg/kg dose only the mAb 338 showed a statistically significant reduction in lung viral titers. It was noted that assessment of the serum concentrations of the antibodies showed a difference in the IgG quantities present in the serum at the time of challenge (FIG. 12, panel C). Taking these concentrations into consideration there was a 2 log reduction in lung titers at a serum concentration similar to what was seen in the experiment with the NL\1\00 virus.

The protection of the upper airway against the NL\1\99 virus was more marked than that seen for the infection with the NL\1\00 virus. Challenge with the NL\1\99) virus in the presence of either mAb 338 or mAb 234 caused statistically significant decreases in the viral titers obtained from the nasal turbinates at the 3 mg/kg dose (FIG. 12, panel B); mAb 234 caused a 2 log reduction and the mAb 338 gave a 2.5 log reduction. These data demonstrate that the antibodies present in the upper airways were more effective against the B type virus than the A type virus. This difference could be correlated to the differences seen in vitro in IC₅₀ values for these antibodies. The IC₅₀ values of mAb 338 are 10 fold lower for the B1 virus relative to the A1 virus and the values for mAb 234 differ by 50 fold when comparing A1 neutralization with B1 neutralization.

It has been established that immunization with a viral vector containing hMPV F protein can induce a potent neutralizing immune response that can protect against hMPV challenge (19, 21). These studies indicate that an immune response, specific to the F protein alone, can neutralize virus in vivo in the absence of cellular or humoral immunity to other hMPV-specific antigens. A recent paper has described the isolation of neutralizing monoclonal antibodies to hMPV that were obtained by immunization with infected cells (14). These antibodies were reactive with the F protein of hMPV and neutralized virus in a PCR based assay. However, the studies did not look for in vivo protection. Thus, the studies so far have not shown that mAbs to F protein alone can protect animals from virus challenge. Additionally, since the earlier study (14) did not use purified antibody the potency of the mAb neutralization in vitro or in vivo has not been established.

In this report it is shown that neutralizing monoclonal antibodies can be obtained from animals immunized with human metapneumovirus F protein and that these antibodies can protect cells from infection in vitro and protect animals from infection in vivo. It has been found that only a small number of antibodies cross-neutralized all 4 hMPV prototypic subgroups even though the conservation of F protein sequence (95%) might have suggested that the majority of antibodies would be pan neutralizing. Many of the antibodies that were isolated were not able to neutralize at least one of the 4 virus types with comparable potency, which suggests that the neutralizing epitopes may be the regions of highest variability, presumably as a result of selective pressures.

A comparison of the differential abilities of the antibodies to neutralize the viral subgroups, and the ability of the mAbs to cross-compete with each other for binding to the F protein, led to the identification of 6 types of epitope. Of these 3 distinct non-overlapping epitopes that are recognized by mAb 1017, mAb 757 and the mAb 967/mAb1025 pair were found. The remaining 3 epitopes have antibodies that recognize one of two independent epitopes or recognize the overlap between these two epitopes (FIG. 10). Monoclonal antibody resistant mutants of the hMPV virus that will allow the determination of the precise location on the F protein sequence to which they bind are being generated.

Two of the antibodies that neutralized all 4 subgroup prototypes at an IC50 of <5 μg/ml, mAb 234 and mAb 338, are good candidates for further study. Both of these antibodies have properties that are similar to the properties of palivizumab that is currently used for the prophylaxis of RSV infection in at risk infants (10,28). A comparison of the neutralization and binding properties of the hMPV antibodies to neutralization and binding properties of palivizumab to it's RSV target are shown in Table 12. Both antibodies, mAb 234 and mAb 338, show high affinity binding to soluble F protein from both an A group and a B group sequence. Both mAb 234 and mAb 338 have kₒₙ rates of 2-8×10⁵ M-1s⁻¹ against both types (A and B) of soluble F protein. These Kₒₙ rates are comparable to the Kₒₙ rate of palivizumab for RSV soluble F protein (1.2×10⁵ M-1s⁻¹)(wu 2005). The k_{off} rates of the mAb 234 and mAb 338 comparable to palivizumab against it's F protein (7×10⁻⁴ s⁻¹). Overall the K_{d} values of the two anti-hMPV mAbs and paluvizumab are less than 10 nM.

TABLE 12
Comparison of anti-hMPV and anti-RSV monoclonals

| | | | | | | In vivo | |
|---|---|---|---|---|---|---|---|
| | In vitro | | | | | 2 log reduction data | |
| | | Kₒₙ | K_{off} | | IC₅₀ | | [IgG] |
| | Virus | (×10⁵) | (×10⁻⁴) | Kd | neutralization | dose | serum |
| mAb | Type | M⁻¹s⁻¹ | M⁻¹ | (nM) | (μg/ml) | (mg/kg) | (μg/ml) |
| mAb 234¹ | A | 7.83 | 3.52 | 4.49 | 1.2-0.6 | 1.0 | 8 |
| | B | 1.92 | 4.63 | 2.41 | 0.2-0.01 | 1.0 | 6 |
| mAb 338¹ | A | 1.58 | 2.76 | 1.74 | 0.8-0.4 | 1.0 | 5 |
| | B | 1.92 | 2.72 | 1.42 | 0.2-0.03 | 0.3 | 4 |
| Palivizumab^{2,3,4} | A | 1.27 | 4.3 | 3.39 | 0.453 | 2.5 | ˜30 |
| | B | ND | ND | ND | 0.06 | 2.5 | ˜30 |

¹Data is derived from this publication
^{2,3,4}Data is from references 10, 28 and personal communication from N. K. Patel.

In comparison to palivizumab the in vitro neutralization capacity of the mAb 234 and mAb 338 antibodies against the A subgroup viruses (IC₅₀ between 1.2 and 0.4 μg/ml) was comparable to the IC₅₀ of palivizumab against it's A group virus at 0.5 μg/ml. The neutralization seen with the mAb 234 and mAb338 antibodies against the B subgroup viruses is somewhat more potent with an IC₅₀ between 0.2 and 0.01 μg/ml for the neutralization of the B2 and B1 subgroups respectively. The increased potency against B group isolates has also been see with palivizumab. Remarkably, the in vivo potency of the hMPV monoclonals was comparable to palivizumab in reducing the viral load in the lungs in the rodent models used. Further testing of the ability of mAb 234 and mAb 338 ability to neutralize a broader range of viral isolates and prevent virus induced pathology is underway. Thus, humanization and optimization of mAbs 338 and 234 may result in viable clinical candidates that can be tested for the the ability to prevent lower respiratory tract disease that results from hMPV infection. Ultimately this could extend our ability to protect those individuals at greatest risk from serious lung infections.

### PUBLICATIONS CITED IN SECTION 7

1) Atkins, J. T., P. Karimi, B. H. Morris, G. McDavid and S. Shim. 2000. Prophylaxis for respiratory syncytial virus with respiratory syncytial virus-immunoglobulin intravenous among preterm infants of thirty-two weeks gestation and less: reduction in incidence, severity of illness and cost. Pediatr Infect Dis. 19, 138-143.

2) Bebbington C, et al. High level expression of a recombinant antibody from myeloma cells using a glutamine synthetase gene as an amplifiable selectable marker. Bio/Technology 1992; 10: 169-175.

3) Biacchesi, S., M. H. Skiadopoulos, G. Boivin, C. T. Hanson, B. R. Murphy, P. L. Collins, and U. J. Buchholz. 2003. Genetic diversity between human metapneumovirus subgroups. Virology. 315,1-9

4) Boivin, G., I. Mackay, T. P. Sloots, S. Madhi, F. Freymuth, D. Wolf, Y. Shemer-Avni, H. Ludewick, G. C. Gray and E. LeBlanc. 2004 Global genetic diversity of human metapneumovirus fusion gene. Emerg Infect Dis. 10, 1154-1157.

5) Crowe, J. E. 2004. Human Metapneumovirus as a Major Cause of Human Respiratory Tract Disease. Pediatr Infecect Dis J. 23, S215-S221.

6) de St Groth, F S and Scheidegger, D. Production of monoclonal antibodies: strategy and tactics Journal of Immunological Methods 35(1980), 1-21.

7) Falsey, A. R., D. Erdman, L. J. Anderson and E. E. Walsh. 2003. Human metapneumovirus infections in young and elderly adults J. Infect. Dis. 187:785-790.

8) the Impact—RSV Study group. 1998 Palivizumab, a humanized respiraratory syncytial virus monoclonal antibody, reduced hospitalization from respiratory syncytial virus infection in high risk infants. Pediatrics 102, 531-537.

9) Ison, M. G. and F. G. Hayden 2002. Viral infections in immunocompromised patients:what's new with respiratory viruses? Curr Opinion Infect Dis 15:355-367.

10) Johnson, S, C. Oliver, G. A. Prince, V. G. Hemming, D. S. Pfarr, S. C. Wang, M. Dormitzer, J. O'Grady, S. Koenig, J. K. Tamura, R. Woods, G. Bansal, D. Couchenour, E. Tsao, W. C. Hall and J. F. Young. 1997. Development of a humanized monoclonal antibody (MEDI-493) with potent in vitro and in vivo activity against respiratory syncytial virus. J Infect Dis. 176:1215-24.

11) Johnsson, B., S. Lofas and G. Lindquist. 1991. Immobilization of proteins to a carboxymethyldextran-modified gold surface for bispecific interaction analysis in surface pasmon resonance sensors. Anal. Biochem. 198, 268-277.

12) Karber, G. 1931. 50% end-point calculation. Arch. Exp. Pathol. Pharmak., 162, 480-483

13) Leung, J., F. Esper, C. Weibel and J. S. Kahn. 2005. Seroepidemiology of human metapneumovirus (hMPV) on the basis of a novel enzyme-linked immunosorbent assay utilizing HMPV fusion protein expressed in recombinant vesicular stomatitis virus J. Clin. Micro. 43:1213-1219.

14) Ma, X., R. Endo, T. Ebihara, N. Ishiguro, H. Ishiko and H. Kikuta. 2005. Production and Characterization of Neutralizing Monoclonal Antibodies Against Hjuman Metapneumovirus F Protein. Hybridoma 24, 201-205.

15) MacPhail, M., J. H. Schickli, R. S. Tang, J. Kaur, C. Robinson, R. A. Fouchier, A. D. Osterhaus, R. R. Spaete and A. A. Halle. 2004 Identification of small-animal and primate models for evaluation of vaccine candidates for human metapneumovirus (hMPV) and implications for hMPV vaccine design. J Gen Virol. 85, 1655-1663.

16) Mejías, A., S. Chávez-bueno and O. Ramillo. 2004. Human Metapneumovirus: a not so new virus. Pediatr Infect Dis J. 23, 1-10.

17) Robinson, J. L., B. E. Lee, N. Bastein and Y. Li. 2005. Seasonality and clinical features of human metapneumovirus infection in children in northern Alberta. J. Med. Virol. 76:98-105.

18) Sinaniotis, C. A. 2004 Viral Pneumoniae in children: incidence and aetiology. Paediatr Respiratory Rev 5, S197-S200.

19) Skiadopoulos, M. H., S. Biacchesi, U. J. Buchholz, J. M. Riggs, S. R. Surman, E. Amaro-Carambot, J. M. McAuliffe, W. R. Elkins, M. St Claire, P. L. Collins and B. R. Murphy. 2004. The two major human metapneumovirus genetic lineages are highly related antigenically, and the fusion (F) protein is a major contributor to this antigenic relatedness. J Virol. 78, 6927-6937.

20) Sorrentino M, Powers T. 2000. Effectiveness of palivizumab: evaluation of outcomes from the 1998 to 1999 respiratory syncytial virus season. The Palivizumab Outcomes Study Group. Pediatr Infect Dis J. 19:1068-71.

21) Tang R S. Schickli J H. MacPhail M. Fernandes F. Bicha L. Spaete J. Fouchier R A. Osterhaus A D. Spaete R. Haller A A. 2003. Effects of human metapneumovirus and respiratory syncytial virus antigen insertion in two 3′ proximal genome positions of bovine/human parainfluenza virus type 3 on virus replication and immunogenicity. Journal of Virology. 77,10819-10828

22) Ulloa-Gutierrez R, Skippen P, Synnes A, Seear M, Bastein N Li Y and Forbes J C 2004. Life-threatening human metapneumovirus pneumonia requiring extracorporeal membrane oxygenation in a preterm infant. Pediatrics 114:517-519.

23) van den Hoogen, B. G., J. C. de Jong, J. Groen, T. Kuiken, R. de Groot, R. A. Fouchier and A. D. Osterhaus. 2001. A newly discovered human pneumovirus isolated from young children with respiratory tract disease. Nature Medicine. 7,719-724.

24) van den Hoogen, B. G., T. M. Bestebroer, A. D. Osterhaus and R. A. Fouchier. 2002. Analysis of the genomic sequence of a human metapneumovirus. Virology. 295,119-132,

25) van den Hoogen, B. G., S. Herfst, L. Sprong, P. A. Cane, E. Forleo-Neto, R. L. de Swart, A. D. Osterhaus and R. A. Fouchier. 2004. Antigenic and genetic variability of human metapneumoviruses. Emerg Infect Dis 10,658-666.

26) Williams, J. V., P. A. Harris, S. J. Tollefson, L. L. Halburrt-Rush, J. Pingsterhaus, K. M. Edwards, P. F. Wright, and J. E. Crowe Jr. 2004. Human metapneumovirus and lower respiratrory tract disease in otherwise healthy infants and children. New Engl. J. Med. 350:443-450.

27) Welliver, R. C. 2003.Review of Epidemiology and Clincal Risk Factors for Severe Respiratory Syncytial Virus (RSV) Infection. J. Pediatr. 143:S112-S117.

28) Wu, H., D. S. Pfarr, Y. Tang, L. L. An, N. K. Patel, J. D. Watkins, W. D. Huse, P. A. Kiener and J. F. Young. 2005. Ultra-potent Antibodies Against Respiratory Syncytial Virus: Effects of Binding Kinetics and Binding Valence on Viral Neutralization. J. Mol. Biol. 350:126-144.

29) Wyde, P. R., S. N. Chetty, A. M. Jewell, G. Boivin and P. A. Piedra. 2003. Comparison of the inhibition of human metapneumovirus and respiratory syncytial virus by ribavirin and immune serum globulin in vitro. Antiviral Research. 60,51-59.

### REFERENCES CITED IN THE SPECIFICATION

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled. Such modifications are intended to fall within the scope of the appended claims.

All references, patent and non-patent, cited herein are incorporated herein by reference in their entireties and for all purposes to the same extent as if each individual publication or patent or patent application was specifically and individually indicated to be incorporated by reference in its entirety for all purposes.

Additionally, U.S. patent application Ser. No. 10/831,780 entitled “Metapneumovirus Strains And Their Use In Vaccine Formulations And As Vectors For Expression Of Antigenic Sequences And Methods For Propagating Virus” filed on Apr. 23, 2004 published as US 2005/0019891 A1 on Jan. 27, 2005 is incorporated herein by reference in its entirety.

What is claimed is:

1. A purified antibody or fragment of an antibody, wherein the antibody or the fragment
(i) immunospecifically binds to a protein consisting of the amino acid sequence of any one of SEQ ID NO:33-116; and (ii) comprises one or more of the following domains:
(a) a VH having an amino acid sequence that is at least 95% identical to SEQ ID NO:2 or 10;
(b) a VH CDR1 having an amino acid sequence that is at least 95% identical to SEQ ID NO:4 or 12;
(c) a VH CDR2 having an amino acid sequence that is at least 95% identical to SEQ ID NO:6 or 14;
(d) a VH CDR3 having an amino acid sequence that is at least 95% identical to SEQ ID NO:8 or 16;
(e) a VL having an amino acid sequence that is at least 95% identical to SEQ ID NO:18 or 26;
(f) a VL CDR1 having an amino acid sequence that is at least 95% identical to SEQ ID NO:20 or 28;
(g) a VL CDR2 having an amino acid sequence that is at least 95% identical to SEQ ID NO:22 or 30; and
(h) a VL CDR3 having an amino acid sequence that is at least 95% identical to SEQ ID NO:24 or 32.

2. A purified antibody or fragment of an antibody, wherein the antibody or the fragment
(i) immunospecifically binds to a protein comprising the amino acid sequence of any one of SEQ ID NO:33-116; and (ii) comprises one or more of the following domains:
(a) a VH having an amino acid sequence of SEQ ID NO:2 or 10;
(b) a VH CDR1 having an amino acid sequence of SEQ ID NO:4 or 12;
(c) a VH CDR2 having an amino acid sequence of SEQ ID NO:6 or 14;
(d) a VH CDR3 having an amino acid sequence of SEQ ID NO:8 or 16;
(e) a VL having an amino acid sequence of SEQ ID NO:18 or 26;
(f) a VL CDR1 having an amino acid sequence of SEQ ID NO:20 or 28;
(g) a VL CDR2 having an amino acid sequence of SEQ ID NO:22 or 30; and
(h) a VL CDR3 having an amino acid sequence of SEQ ID NO:24 or 32.

3. The antibody of claim 1 or 2, wherein the antibody is a monoclonal antibody.

4. The antibody of claim 1 or 2, wherein the antibody is a chimeric antibody, a humanized antibody, or a fully human antibody.

5. The antibody of claim 1 or 2, wherein the antibody neutralizes mammalian metapneumovirus at an IC₅₀ of between 0.05 microgram/milliliter and 2 microgram/milliliter.

6. The antibody of claim 1 or 2, wherein the antibody is mAb234 or mAb338.

7. The antibody of claim 1 or 2, wherein the antibody or fragment comprises 2, 3, 4, 5, 6, 7 or all of the domains listed in claim 1 or 2.

8. The antibody of claim 1 or 2, wherein the antibody or fragment comprises domains (a), (b), (c), and (d) listed in claim 1 or 2.

9. The antibody of claim 1 or 2, wherein the antibody or fragment comprises domain (a) listed in claim 1 or 2.

10. The antibody of claim 1 or 2, wherein the antibody or fragment comprises domain (b) listed in claim 1 or 2.

11. The antibody of claim 1 or 2, wherein the antibody or fragment comprises domain (c) listed in claim 1 or 2.

12. The antibody of claim 1 or 2, wherein the antibody or fragment comprises domain (d) listed in claim 1 or 2.

13. The antibody of claim 1 or 2, wherein the antibody or fragment comprises domain (e) listed in claim 1 or 2.

14. The antibody of claim 1 or 2, wherein the antibody or fragment comprises domain (f) listed in claim 1 or 2.

15. The antibody of claim 1 or 2, wherein the antibody or fragment comprises domain (g) listed in claim 1 or 2.

16. The antibody of claim 1 or 2, wherein the antibody or fragment comprises domain (h) listed in claim 1 or 2.

17. The antibody of claim 1 or 2, wherein the antibody binds immunospecifically to an F protein of RSV.

18. The antibody of claim 17, wherein the antibody binds immunospecifically to any one of the amino acid sequences of SEQ ID NO:132-154.

19. A pharmaceutical composition comprising an effective amount of the antibody of claim 1 or 2 and a pharmaceutically acceptable carrier.

20. A method for preventing, treating, managing, or ameliorating the symptoms of an infection with a mammalian metapneumovirus, said method comprising
administering an effective amount of the pharmaceutical composition of claim 19.

21. A method for preventing, treating, managing, or ameliorating the symptoms of an infection with respiratory syncytial virus, said method comprising administering an effective of the pharmaceutical composition of claim 19.

22. The method of claim 20, wherein the mammalian metapneumovirus is a human metapneumovirus.

23. The method of claim 21, wherein the respiratory syncytial virus is a human respiratory syncytial virus.

24. The antibody of claim 1 or 2, wherein the antibody binds immunospecifically to an F protein of APV.

25. The antibody of claim 1 or 2, wherein the antibody binds immunospecifically to an F protein of APV.
